(19) United States
(12) Patent Application Publication
(10) Pub. No.: US 2023/0111600 A1
(43) Pub. Date: Apr. 13, 2023

(54) POLYMERIC FATTY ACID COMPOUNDS FOR THE TREATMENT OF FIBROUS AMINO ACID-BASED SUBSTRATES, ESPECIALLY HAIR

(71) Applicant: MOMENTIVE PERFORMANCE MATERIALS GMBH, Leverkusen (DE)

(72) Inventors: Roland WAGNER, Bonn (DE); Katharina STREICHER, Leverkusen (DE); Laura ROHMANN, Düsseldorf (DE); Christian WENSKE, Solingen (DE); Shrutisagar Dattatraya HAVELI, White Plains, NY (US)

(21) Appl. No.: 17/786,081

(22) PCT Filed: Dec. 15, 2020
(86) PCT No.: PCT/IB2020/001036

§ 371 Date: Jun. 16, 2022

Related U.S. Application Data
(60) Provisional application No. 62/949,005, filed on Dec. 17, 2019.

(51) Int. Cl. A61K 8/41; A61Q 5/12; A61Q 19/00; C07C 219/06; A61Q 5/02; A61Q 5/06

(52) U.S. Cl. CPC A61K 8/416; A61Q 5/12; A61Q 19/00; C07C 219/06; A61Q 5/02; A61Q 5/06

(57) ABSTRACT

The present invention is directed at mono-, di- or polyquaternary ammonium compounds of the formula (I) wherein x is 1 to 50, and F can be the same or different and is represented by the general formula (II) wherein at least one of the optionally substituted and optionally functional-group-containing hydrocarbon radicals R¹, R², R³, R⁴ or R⁵ contains at least one estolide moiety comprising two or more ester or amide moieties. The invention also relates to a process for the synthesis of such compounds, the use of the compounds in cosmetic formulations for skin and hair care, cosmetic compositions for the treatment of fibers, and compositions containing one or more of the compounds for the treatment of hair.

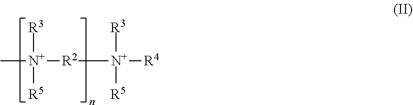

### FIELD OF THE INVENTION

This invention relates to polymeric fatty acid compounds, a process for their production, compositions containing the compounds, and the use of the compounds in cosmetic compositions comprising the same for skin and hair care, in particular, hair care compositions, and their use for the treatment of hair.

### BACKGROUND OF THE INVENTION

Hair generally can be straight, wavy, curly, kinky or twisted. A human hair includes three main morphological components, the cuticle (a thin, outer-most shell of several concentric layers), the cortex (the main body of the hair), and, in case of higher diameter hair, the medulla (a thin, central core). The cuticle and cortex provide the hair strand's mechanical properties, that is, its tendency to have a wave, curl, or kink. A straight hair strand can resemble a rod with a circular cross-section, a wavy hair strand can appear compressed into an oval cross-section, a curly strand can appear further compressed into an elongated ellipse cross-section, and a kinky hair strand cross-section can be flatter still.

The primary component of hair is the cross-linked, alpha-helix protein keratin. Keratins are intermediate filament proteins found specifically in epithelial cells, e.g. human skin and hair, wool, feathers, and nails. The α-helical type I and II keratin intermediate filament proteins (KIFs) with molecular weights around 45-60 kDa are embedded in an amorphous matrix of keratin-associated proteins (KAPs) with molecular weights between 20 to 30 kDa (M. A. Rogers, L. Langbein, S. Praetzel-Wunder, H. Winter, J. Schweizer, J. Int Rev Cytol. 2006; 251:209-6); both intra- and intermolecular disulfide bonds provided by cystines contribute to the cytoskeletal protein network maintaining the cellular scaffolding. In addition to the disulfide cross-links ionic bonding or salt bridges which pair various amino acids found in the hair proteins contribute to the hair strand's outward shape.

It is known in the art that hair can be treated with functionalized silicones and hydrocarbons which deliver one or more cosmetic benefits, such as conditioning, shine and UV protection as well as color retention. Typically, these silicones and hydrocarbon-based derivatives are physically deposited on the fiber surface (cuticle) and therefore responsible for the outward appearance of the hair, i.e. smoothness, silkiness, friction, alignment and combability.

Advanced silicone derivatives are generally regarded as high performing materials with respect to attributes such as smooth and silky hair feel, friction reduction, eased combability and hair color protection. Respective quaternized silicones are described in prior art disclosures, i.e. in U.S. Pat. No. 4,891,166, EP 282720, US 2008027202, U.S. Pat. Nos. 6,730,766, 6,240,929, WO 02/10257, WO 02/10259, WO 2004/069137, WO 2013/148629, WO 2013/148635, WO 2013/148935.

Hydrocarbon-based conditioning agents are also widely used. Typically, mono quaternary ammonium compounds are mono-long alkyl—tri short alkyl quaternized ammonium salts or di-long alkyl—di short alkyl quaternized ammonium salts wherein one or two alkyl substituents are selected from an aliphatic group of from about 8 to about 30 carbon atoms or an aromatic, alkoxy, polyoxyalkylene, alkylamido, hydroxyalkyl, aryl or alkylaryl group having up to about 30 carbon atoms; the other alkyl groups are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and the counter ion is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. Alternatively, these mono quaternary ammonium compounds are saturated or unsaturated fatty acid-based mono-fatty ester and di-fatty ester quats as well as fatty amido quats having 10 to 24 carbon atoms in the alkyl chain(s). Details on these materials containing quaternary ammonium groups are disclosed for example in US 2009/0000638, WO 2012/027369, US 2013/259820 and U.S. Pat. Nos. 5,880,086, 6,465,419, 6,462,014, 6,323,167, 6,037,315, 5,854,201, 5,750,490, 5,463,094, US 2003/013627.

Di-quaternized hydrocarbons are also known. Typically, these gemini quats are based on C8 to C20 alkyl or fatty chains (D. Shukla et. al., Cationic Gemini Surfactants: A Review, Journal of Oleo Science 2006, Vol. 55, Nr. 8, 381-390; M. J. Rosen et. al. Langmuir (2001), 17, 6148-6154).

Di-quaternized hydrocarbons based on an alternating copolyester of castor oil and different dicarboxylic acids are described in US 2003/0007950 and U.S. Pat. No. 6,972,123.

A castor oil precursor was used to synthesize a material containing three quat groups (EP 0283994, A. Baydar et. al., International Journal of Cosmetic Science (1991), 13(4), 169-90). Dimers of fatty acids were used to synthesize polyquaternary fatty acid dimer copolymers (U.S. Pat. No. 6,982,078).

WO 2004/093834 describes hydrocarbon based mono quaternary compounds for personal care applications. These compounds mandatorily contain linkers having the structure —CH₂CH₂O—EOₓ—PO_{y}—. Polymerized fatty acids were proposed as hydrophobic tails.

There has been a need for efficient compounds for the treatment of fibrous amino acid based substrates, especially hair which can be synthesized in a straight forward, cost efficient and flexible way, largely based on sustainable raw materials, which are easy to formulate and easy to use, yielding long term stable formulations even in the presence of other performance ingredients and which are useful for the conditioning of hair, for an improved dry and wet combability of hair, the smoothness and a pleasant alignment of hair. In particular, benefits regarding an improved wet and dry combability close to silicone based conditioning agents should be achieved.

The present inventors found that new polymeric fatty acid-based mono-, di- and poly-quaternary compounds, i.e. mono-, di- and polyquaternary compounds comprising estolide structures, and aqueous compositions comprising such compounds are suitable to satisfy the above need. The present invention accordingly provides new polymeric fatty acid based mono-, di- and poly-quaternary estolide compounds, aqueous compositions comprising the same, cosmetic compositions comprising the same, in particular, hair care compositions, and their use for the treatment of hair, which polymeric fatty acid based mono-, di- and poly-quaternary estolide compounds can be synthesized in a straightforward, cost-efficient and flexible way, largely based on sustainable raw materials, are easy to formulate and to use, and are useful for the conditioning of hair, for an improved dry and wet combability of hair, the smoothness and a pleasant alignment of hair.

### SUMMARY OF THE INVENTION

In accordance with the present invention, a compound of the formula:

R¹(—F)ₓ (I)

is provided, wherein
x is 1 to 50, preferably 2 to 50,
R¹ is selected from x-valent, optionally substituted hydrocarbon radicals which have up to 1000 carbon atoms, preferred 2 to 300 carbon atoms, more preferred 3 to 200 carbon atoms, even more preferred 3 to and 150 carbon atoms, specifically 3 to 50 carbon atoms, more specifically 3 to 20 carbon atoms, and may contain optionally one or more groups selected from —O—, —NH—, —C(O)—, —C(S)—, tertiary amino groups

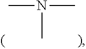

and can be substituted by one or more groups selected from OH groups and halide groups, and
F can be the same or different and is represented by the general formula (II)

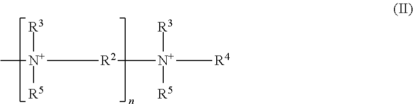

wherein the groups F bind to a carbon atom of R¹, and
n is independently 0 to 100,
R² can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups

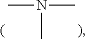

and can be substituted with one or more groups selected from OH groups and halide groups,
R³, R⁴, R⁵ can be the same or different and are selected from hydrogen and optionally substituted straight-chain, cyclic or branched, saturated, unsaturated or aromatic hydrocarbon radicals which have up to 1000 carbon atoms, which optionally contain one or more groups selected from —O—, —NH—, —C(O)—, —C(S)—, tertiary amino groups

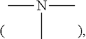

quaternary ammonium groups

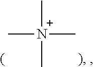

and can be substituted with one or more groups selected from OH groups and halide groups,
wherein R³, R⁴, R⁵ each bind with a carbon atom to the nitrogen atom,
and preferably R³, R⁴, R⁵ are not hydrogen,
the counter ions A⁻ of the ammonium ions are selected from mono to trivalent inorganic and mono- to 30000-valent, preferably mono- to kiliavalent organic anions, and
at least one of R¹, R², R³, R⁴, R⁵ present in the cationic structure of the general formulas (I) and (II) contains at least one moiety of the formulas (III) or (IV):

(—X—C(O)—R⁶)ₘ—X—C(O)— (III) or

(—C(O)—X—R⁶)ₘ—C(O)—X— (IV),

wherein
m=1 to 20,

### X is 0 or NR,

R¹¹ is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

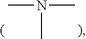

and can be substituted with one or more hydroxyl and halide groups,
R⁶ is independently selected from optionally substituted straight-chain, cyclic or branched, saturated or unsaturated hydrocarbon radicals which have 1 to 36 carbon atoms, with the proviso that at least one R⁶ has more than 6 carbon atoms, and
that for x=1
R¹, R³, R⁴, R⁵ do not bind through —OCH₂CH₂— to the nitrogen atom of the group

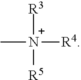

### DETAILED DESCRIPTION OF THE INVENTION

According to the invention, estolides are natural and synthetic compounds, in particular derived from fats and oils, more specifically from the fatty acid compounds typically obtainable by hydrolysis of oils and fats.

The estolide structure is identified by the secondary ester linkage of one fatty acyl molecule to the alkyl backbone of another fatty acid fragment. The terms “fatty acid” and “fatty acyl molecule” seem to imply that the individual residue needs to be derived from a component of a fat, which is not the case. The term “fatty acid” herein refers to carboxylic acids with chain-shaped organyl groups, in particular unbranched aliphatic monocarboxylic acids. Fatty acids differ from each other by their number of carbon atoms (chain length) and, when referring to unsaturated fatty acids, the number and position of double bonds. Fatty acids may be classified as short chain fatty acids with up to 7 carbons atoms, middle chain fatty acids with 8 to 12 carbon atoms, long chain fatty acids with 13 to 21 carbon atoms, and very long chain fatty acids with more than 22 carbon atoms.

According to the invention, in general the group “—O—” represents an ether group, which also includes the presence of an epoxide moiety, which is a tri-membered cyclic ether group. Accordingly, the groups defined above as optionally comprising the group “—O—” may contain epoxy groups. This applies in particular to the residues R³, R⁴, and R⁵ as defined above, which may include a terminal epoxy group.

According to the invention, the residue R¹ is x-valent, wherein x is 1 to 50, preferably 2 to 50, which indicates that the residue R¹ bears x residues F as defined by the general formula (II). Accordingly, the term “x-valent” does not refer to or restrict the number of optional further substituents other than F of the residue R¹, which can be hydroxyl groups and halide groups.

According to the invention the wording “optionally substituted hydrocarbon radical” that may contain optionally one or more specific groups and can be substituted by one or more specific groups refers to an organyl radical which is linked to one or more further groups via at least one of its carbon atoms, wherein the hydrocarbyl structure of the radical may be interrupted by the specific groups as defined to be contained, and one or more hydrogen atoms of the hydrocarbyl group can be substituted by the substituent groups as indicated.

In case of R¹, for example, one or more hydrogen atoms may be substituted by a hydroxyl group or by an halide substituent, i.e. by a fluoro, chloro, bromo or iodo substituent.

Further, as the optionally substituted hydrocarbon radical R¹ specifically may contain one or more groups selected from —O—, —NH—, —C(O)—, —C(S)— and tertiary amino groups,

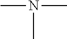

the hydrocarbyl structure of a R¹ group may be interrupted by these groups or combinations thereof. Accordingly, the residue may contain ester groups, carboxyl groups, amide groups, ether groups, amino groups, carbonyl groups, thione groups, thio carboxylate groups, thio ester groups, carbamate groups, urethane groups, epoxide groups and all other groups as specified for this radical, and combinations thereof. The same principle applies to the optionally substituted hydrocarbon radicals R², R³, R⁴, R⁵, R⁶, and R¹¹.

The hydrocarbyl structure of R¹, which is x-valent regarding the residues F, is preferably selected from the group consisting of linear, branched or cyclic alkyl or alkylene groups, linear, branched or cyclic alkenyl or alkenylene groups, linear, branched or cyclic alkynyl or alkynylene groups, linear, branched or cyclic alkaryl or alkarylene groups, linear, branched or cyclic aralkyl or aralkylene groups and linear, branched or cyclic aryl or arylene groups, for instance phenyl or phenylene, benzyl or benzylene or tolyl or tolylene groups, in particular from such groups having 1 to 30 carbon atoms.

More preferably, the x-valent R¹ radical is selected from alkyl or alkylene groups, which may be selected from the group consisting of linear, branched and cyclic alkyl or alkylene groups or groups combining linear and cyclic alkyl or alkylene structures, or groups combining branched and cyclic structures, in particular from linear C1-C22 alkyl groups such as methyl and methylene, ethyl and ethylene, n-propyl and n-propylene, n-butyl and n-butylene, n-pentyl and n-pentylene, n-hexyl and n-hexylene, n-heptyl and n-heptylene or n-octyl and n-octylene groups, branched C1-C22 alkyl and alkylene groups such as iso-propyl and iso-propylene, iso-butyl and iso-butylene, tert-butyl and tert-butylene, iso-pentyl and iso-butylene, tert-pentyl and tert-pentylene, neo-pentyl and neo-pentylene, and 2-ethylhexyl and 2-ethylhexylene groups, and from cyclic C3-C22 alkyl groups such as cyclopropyl or cyclopropylene, cyclobutyl and cyclobutylene, cyclopentyl and cyclopentylene, cyclohexyl and cyclohexylene, and cycloheptyl or cycloheptylene groups.

In case x is >1, there is no limitation regarding at which C-atoms of the hydrocarbyl radicals the groups F are bonded to R¹. Regarding the presence of functional groups optionally contained in R¹ and optional substituents, it is preferred that R¹ is derived from glycidyl compounds, glycerol and glycerol derivatives, in particular glycidol, glycerol, glycerol diglycidyl ether, diglycidyl ether and polyglycerol compounds, or when R¹ is a linear alkylene group, in particular an alkylene group not bearing further substituents in addition to the F groups.

As stated above, it is particularly preferred when R¹ is derived from glycerol diglycidyl ether, which means that R² is formed by opening of the epoxide rings of glycerol diglycidyl ether by N atoms then forming the quaternary N atoms adjacent to the R¹ group in the compounds according to the invention. In the same manner, it is preferred when R¹ is derived from diglycidyl ether, diglycerol diglycidyl ether, triglycerol diglycidyl ether, polyglycerols terminated with glycidyl units, and poly(alkylene oxide) compounds terminated with glycidyl units, in particular poly(ethylene oxide)s terminated with glycidyl units, poly(propylene oxide)s terminated with glycidyl units, and poly(butylene oxide)s terminated with glycidyl units.

It is also preferred when R¹ is formed from compounds obtained by esterification of polyols, in particular diol compounds such as α,ω-diols or α,ω-dihydroxypolyethers, more particular dihydroxy-terminated poly(ethylene oxide), dihydroxy-terminated poly(propylene oxide) or dihydroxy-terminated poly(butylene oxide) with ω-halocarboxylic acids, in particular ω-chloro acetic acid or ω-chloropropanoic acid. Latter compounds form R¹ by substitution of the chloro substituents by the N-atoms of the F groups adjacent to the R¹ group.

According to this, it is preferred when R¹ is a C3-C50 alkylene group containing one or more internal ether or ester groups, and it is particularly preferred when R¹ is such alkylene group bearing hydroxyl substituents.

It is most preferred when R¹ is a linear C1-C8 alkylene group without further substituents or functional groups, or when R² is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units.

According to the invention, the term “optionally substituted hydrocarbon residue” does not impose any further restrictions on the radicals, and accordingly they are limited by the groups which can be optionally contained or present as substituents, the number of carbon atoms of the residues as specified, and the way they are bonded to other structural moieties of the compound according to the invention as defined by formula (I), formula (II), formula (III), formula (IV) or any further formula used to define an embodiment according to the invention. For example, in case of R² the term “divalent” refers to R² being bond to two quaternary N atoms according to formula (II), but does not limit the presence of further other substituents as defined for R².

The residues R², R³, R⁴, R⁵, R⁶, and R¹¹ thus can be optionally substituted straight-chain, cyclic or branched, saturated, unsaturated or aromatic hydrocarbon radicals, wherein R² and R⁶ are divalent radicals, while R³, R⁴, R⁵ and R¹¹ are monovalent radicals.

R² is, according to formula (II), bonded to two different quaternary N atoms, and R⁶ is bonded to a carbonxyl group of an carboxylate or amide moiety on the one side, and to a group X which can be an O atom of a carboxylate group or an NR¹¹ group of an amide group on the other side, for instance by the definition of formula (III) or (IV), but also to further formulas according to further embodiments according to the invention.

The radicals R³, R⁴, R⁵ and R¹¹ are monovalent radicals which can be the same or different and are selected from hydrogen and optionally substituted straight-chain, cyclic or branched, saturated, unsaturated or aromatic hydrocarbon radicals which have up to 1000 carbon atoms, and can thus represent linear, i.e. straight-chained, cyclic or branched alkyl groups, linear, cyclic or branched alkenyl groups, linear, cyclic or branched alkynyl groups, linear, cyclic or branched alkaryl groups, linear, cyclic or branched aralkyl groups and aryl groups, for instance phenyl, benzyl or tolyl groups, in particular groups having 1 to 30 carbon atoms, and optionally the aforementioned groups may be substituted with OH or halide groups, and may optionally contain one or more groups selected from —O—, —NH—, —C(O)—, —C(S)—, tertiary amino groups

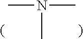

and quaternary ammonium groups

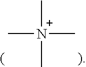

Preferably, the radicals R³, R⁴, R⁵ and R¹¹ are selected from alkyl groups, which may be selected from the group consisting of linear, branched and cyclic alkyl groups or groups combining linear and cyclic alkyl motifs, or structures combining branched and cyclic structures, in particular from linear C1-C22 alkyl groups such as methyl, ethyl, n-propyl, n-butyl, n-pentyl, n-hexyl, n-hexyl, n-heptyl or n-octyl groups, branched C1-C22 alkyl groups such as iso-propyl, iso-butyl, tert-butyl, iso-pentyl, tert-pentyl, neo-pentyl and 2-ethylhexyl groups, and from cyclic C3-C22 alkyl groups such as cyclopropyl, cyclobutyl, cyclopentyl, cyclohexyl and cycloheptyl groups, more preferably the radicals R³, R⁴, R⁵ and R¹¹ are selected from methyl, ethyl, isopropyl, tert-butyl, cyclopentyl or cyclohexyl groups, most preferably from methyl.

The radicals R² according to the invention can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups

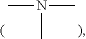

and can be substituted with one or more groups selected from OH groups and halide groups, and are preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 100 carbon atoms, each optionally containing one or more functional groups as indicated above.

More preferably, the R² radical is selected from an alkylene groups, which may be selected from the group consisting of linear, branched and cyclic alkylene groups or groups combining linear and cyclic alkylene structures, or groups combining branched and cyclic structures, in particular from linear C1-C50 alkyene groups such as methylene, ethylene, n-propylene, n-butylene, n-pentylene, n-hexylene, n-heptylene or n-octylene groups, branched C4-C50 alkylene groups such as iso-propylene, iso-butylene, tert-butylene, tert-pentylene, neo-pentylene, 2-ethylhexylene groups, and from cyclic C3-C22 alkyl groups such as cyclopropylene, cyclobutylene, cyclopentylene, cyclohexylene, and cycloheptylene groups.

There is no limitation regarding at which C-atoms of the hydrocarbyl radicals the quaternary N atoms are bonded to R².

Regarding the presence of functional groups optionally contained in R² and optional substituents, it is preferred that R² is derived from glycidyl compounds, glycerol and glycerol derivatives, in particular glycidol, glycerol diglycidyl ether, diglycidyl ether and polyglycerol compounds, or when R² is a linear alkylene group, in particular an alkylene group not bearing further substituents in addition to the quaternary N atoms.

As stated above, it is particularly preferred when R² is derived from glycerol diglycidyl ether, which means that R² is formed by opening of the epoxide rings of glycerol diglycidyl ether by N atoms then forming the quaternary N atoms adjacent to the R² group in the compounds according to the invention. In the same manner, it is preferred when R² is derived from diglycidyl ether, diglycerol diglycidyl ether, triglycerol diglycidyl ether, polyglycerols terminated with glycidyl units, and poly(alkylene oxide) compounds terminated with glycidyl units, in particular poly(ethylene oxide)s terminated with glycidyl units, poly(propylene oxide)s terminated with glycidyl units, and poly(butylene oxide)s terminated with glycidyl units. It is also preferred when R² is formed from compounds obtained by esterification of diol compounds such as α,ω-diols or α,ω-dihydroxypolyethers, in particular dihydroxy-terminated poly(ethylene oxide), dihydroxy-terminated poly(propylene oxide) or dihydroxy-terminated poly(butylene oxide) with ω-halocarboxylic acids, in particular ω-chloro acetic acid or ω-chloropropanoic acid. Latter compounds form R² by substitution of the chloro substituents by the N-atoms adjacent to the R² group.

According to this, it is preferred when R² is a C3-C50 alkylene group containing one or more internal ether or ester groups, and it is particularly preferred when R² is such alkylene group bearing hydroxyl substituents.

It is most preferred when R² is a linear C1-C8 alkylene group without further substituents or functional groups, or when R² is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units.

The radicals R⁶ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 100 carbon atoms, each optionally containing one or more functional groups as indicated above.

More preferably, the R⁶ radical is selected from linear alkylene groups and linear alkenylene groups, in particular from linear C6-C24 alkyene groups such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

There is no limitation regarding at which C-atoms of the hydrocarbyl radicals the adjacent group C(O) group and X group are bonded to R⁶.

However, R⁶ is preferably derived from a hydroxycarboxylic acid bearing one or more hydroxylic groups, more preferably from a monohydroxy carboxylic acid, most preferably from C7-C25 fatty acids bearing one hydroxyl group as substituent. Accordingly, R⁶ preferably represents the alkylene or alkenylene chain of such carboxylic acids. For instance, if R⁶ is derived from ricinoleic acid

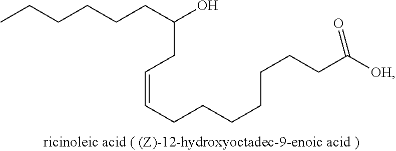 [SMILES: CCCCCCC(O)C/C=C\CCCCCCCC(=O)O]

then R⁶ represents a 1,11-heptadec-8-enyl radical

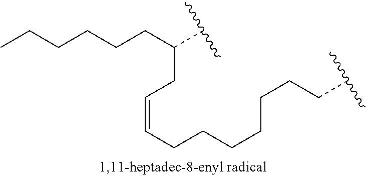

wherein “1,11” indicates the positions in which the radical is bonded to the adjacent groups X and C(O).

The number m of the R⁶-containing repeating units (—X—C(O)—R⁶) or (—C(O)—X—R⁶) of the at least one moiety present in the cationic structure of the general formula (I) as defined by formula (III) or formula (IV) is from 1 to 20, preferably from 1 to 15, 1 to 12, 1 to 10, 1 to 8, or from 2 to 20, from 3 to 20, from 4 to 20, from 5 to 20, specifically 1, 2, 3, 4, 5, 6, 7, 8, 9, or 10.

Preferred examples for the residue R¹ are C3-C18 hydroxy-group-substituted polyether radicals, in particular glycerol-based polyether radicals, and C1-C8 linear alkyl or alkylene groups.

Herein, the term polyether comprises in particular poly(alkylene oxide)-derived compounds, wherein the alkylene groups of the repeating units are independently selected from C1-C8 alkylenes.

Preferred examples for the residue R² are linear C1-C8 alkylene radicals, more preferably ethylene, propylene, butylene, pentylene, hexylene and heptylene, most preferably propylene and hexylene.

Preferred examples for the residues R³, R⁴ and R⁵ are linear C1-C8 alkyl groups and linear alkyl groups containing one or more moieties of the formula (III) or (IV), wherein m is preferably 2 to 6, most preferably R³, R⁴ and R⁵ are independently selected from methyl groups and alkyl groups containing one or more moieties of the formula (III).

Preferred examples for R⁶ are the structures derived from a corresponding hydroxyl carboxylic acid by abstraction of the carboxylate group and one OH group, wherein the hydroxyl carboxylic acid is preferably selected from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid, or dihydroxy carboxylic acids, in particular 2,2′-di-hydroxymethyl propanoic acid, 9,10-dihydroxy stearic acid, or polyhydroxy carboxylic acids, in particular gluconic acid. Most preferably, R⁶ is derived in the above-stated manner from lesquerolic acid or ricinoleic acid. In both cases the naturally occurring enantiomers of the compounds, i.e. (9Z,12R)-12-hydroxyoctadec-9-enoic acid obtained by saponification or fractional distillation of hydrolysed castor oil, which is the seed oil of the castor plant, and (11Z, 14R)-14-hydroxyicos-11-enoic acid as isolated from Paysonia and Physaria species, are particularly preferred. However, the racemates, the S enantiomers as well as the E-configured isomers of the compounds, the racemates, the enantiomers and any possible mixture thereof are also preferred according to the invention.

Preferred examples for R¹¹ are C1-C10 alkyl groups, in particular methyl, ethyl, n-propyl, iso-propyl, n-butyl, iso-butyl, tert-butyl, n-pentane and n-hexane groups, cyclopentyl groups and cyclohexane groups, C2-C10 alkenyl groups, in particular vinyl groups and allyl groups, and C6-C12 aromatic groups, in particular phenyl groups, tolyl groups, and benzyl groups, wherein each of the named groups may be substituted by hydroxyl groups or halide groups.

According to the invention, counter ions A⁻ of the ammonium ions according to the invention are selected from mono- to trivalent inorganic or mono- to 30000-valent, preferably mono- to kiliavalent organic anions.

Therein, the counter anions A⁻ are preferably selected from a group consisting of halide anions, such as chloride, bromide, iodide, inorganic oxoacid anions, such as sulphate and phosphate, phosphonate, sulphonate, methosulphate, carboxylate anions, such as acetate, propionate, lactate, octanoate, 2-ethyl-hexanoate, dodecanoate, hexadecanoate, octadecanoate, oleate, ricinoleate, 12-hydroxy-octadecanoate, succinate, maleate, tartrate, polyethercarboxylates, polymeric fatty acid carboxylates of the type

R¹[(—C(O)—X—R⁶)ₘ—C(O)—X—R⁷]ₓ or
R¹[(X—C(O)—R⁶)ₘ—X—C(O)—R⁷]ₓ, wherein either R¹ or at least one of R⁷, or both R¹ and at least one of R⁷ bear one or more carboxylate groups,
preferably with X=O,
in particular
linear polymeric fatty acid carboxylates of the type
—O—C(O)—R⁶(—X—C(O)—R⁶)ₘ₋₁—X—C(O)—R⁷, preferably
—O—C(O)—R⁶—(O—C(O)—R⁶)ₘ—O—C(O)—R⁷,
i.e. derived from linear poly fatty acid structures, such as

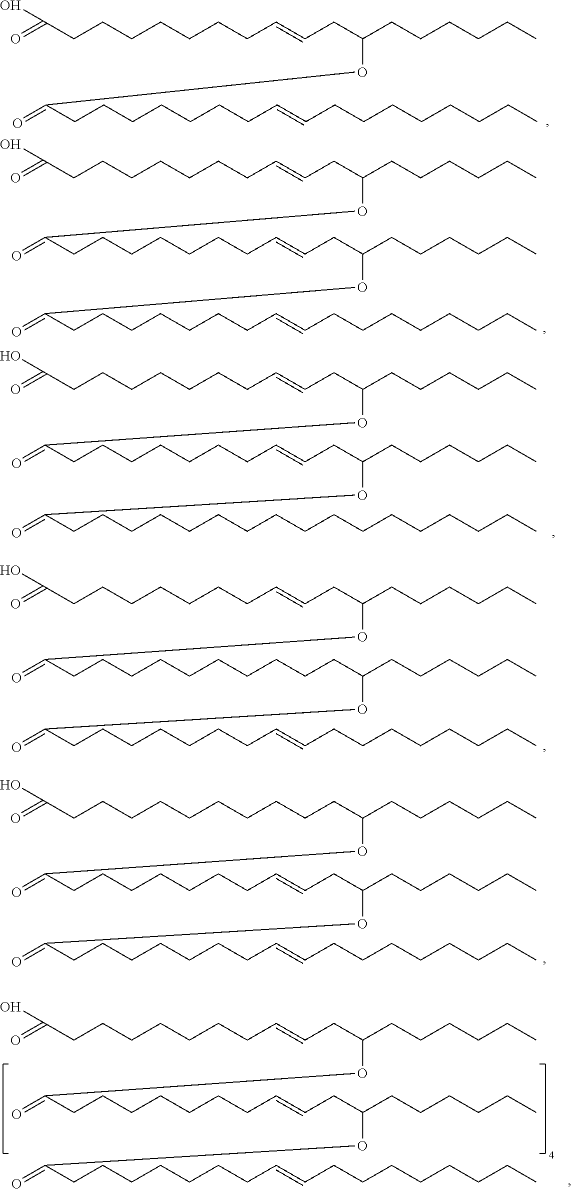

branched linear polymeric fatty acid carboxylates,
i.e. derived from branched poly fatty acid structures, such as

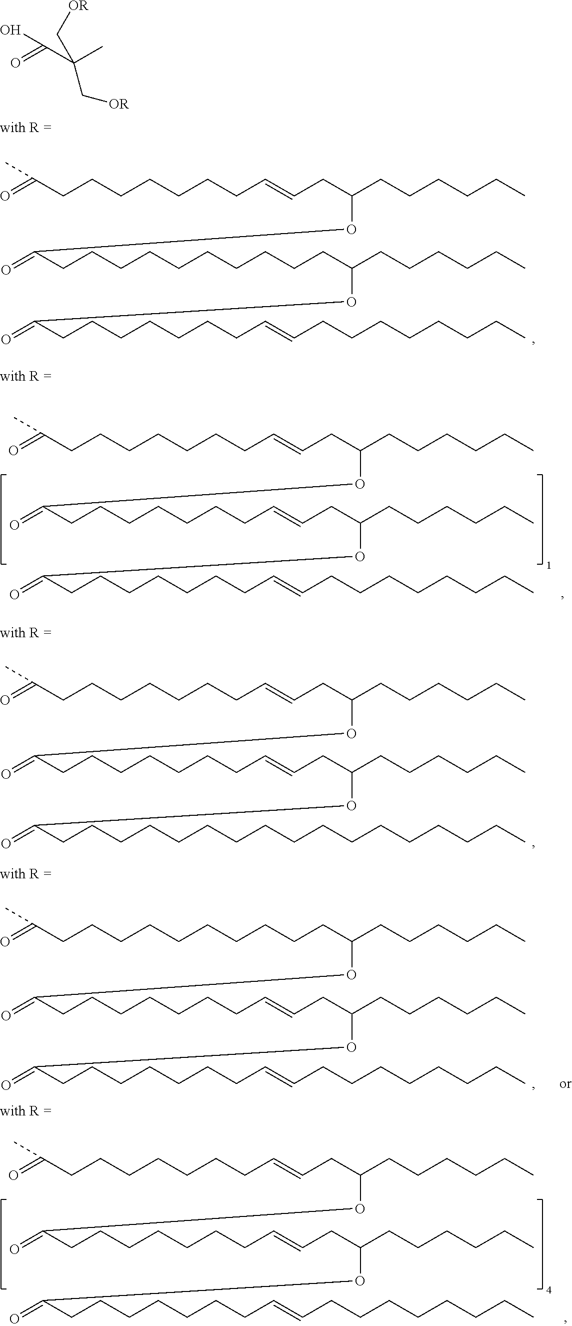

or branched linear polymeric fatty acid carboxylates derived from partial esters of polyfunctional carboxylic acids, in particular of the dicarboxylic acids succinic acid and maleic acid, with castor oil or lesquerella oil, such as

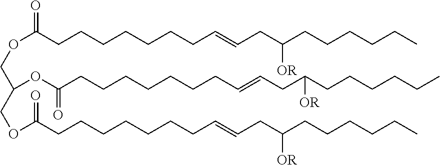

with one

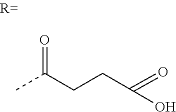

and the remaining two

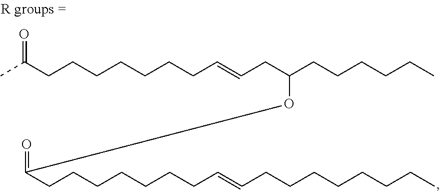

dendritic polymeric fatty acid carboxylates,
i.e. derived from dendritic poly fatty acid structures, such as

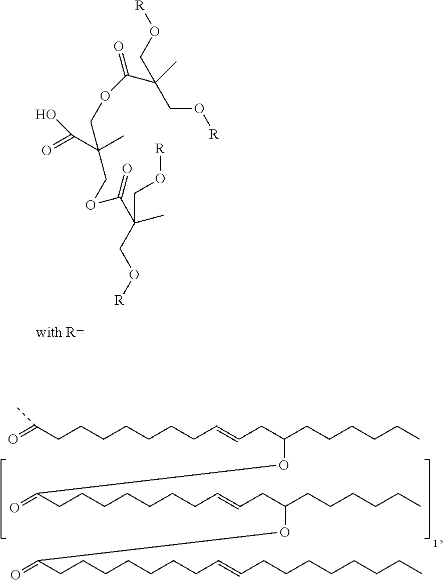

or of the types
X—R⁶(—C(O)—X—R⁶)ₘ₋₁—C(O)—X—R⁷ or
R⁶(—C(O)—X—R⁶)ₘ₋₁—C(O)—X—R⁷,
wherein in the two latter types the R⁷ group bears at least one anionic carboxylate group,
or of the type
R¹[(—C(O)—X—R₆)ₘ—C(O)O⁻]ₓ, such as

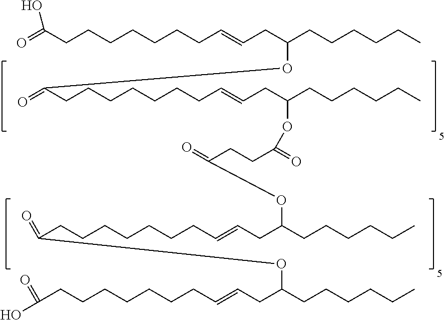

wherein X, R¹, R⁶, m and x are as defined above and
R⁷ is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, tertiary amino groups

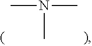

quaternary ammonium groups

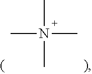

and which can be substituted with OH groups groups or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group,
and wherein the counter ions A⁻ of this group are preferably mono- to pentacontavalent, more preferably mono- to decavalent, even more preferably mono- to pentavalent, most preferably pentavalent, tetravalent, trivalent, divalent or monovalent anions,
or the counter anions are selected from the group consisting of carboxylate anions based on poly (acrylic acid) homo- and co-polymers,
i.e. carboxylates derived from poly acrylic acid homopolymers

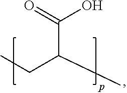

wherein p=2 to 10000, preferred 10 to 10000, more preferred 100 to 10000, even more preferred 1000 to 10000,
carboxylate anions derived from poly acrylic acid copolymers,
i.e. containing non-reactive comonomers, for example

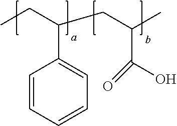

with
a=2 to 10000, preferred 10 to 10000, more preferred 100 to 10000, even more preferred 1000 to 10000
b=2 to 10000, preferred 10 to 10000, more preferred 100 to 10000, even more preferred 1000 to 10000 (valid for all comonomers), wherein
the copolymers can have a blockwise or random distribution of the comonomer units, or carboxylate anions derived from poly acrylic acid copolymers
containing comonomers providing OH and amine functions, which can be functionalised via additional ester or amide bonds, in particular with fatty acids or poly fatty acids, for example

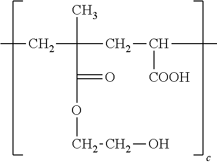

with c=2 to 10000, preferred 10 to 10000, more preferred 100 to 10000, even more preferred 1000 to 10000,
or carboxylate anions derived from poly acrylic acid copolymers containing carboxylic acid functions-containing comonomers, wherein
the copolymers can have a blockwise or random distribution of the comonomer units, for example

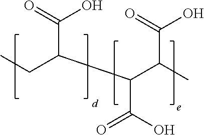

with
d=2 to 10000, preferred 10 to 10000, more preferred 100 to 10000, even more preferred 1000 to 10000
e=2 to 10000, preferred 10 to 10000, more preferred 100 to 10000, even more preferred 1000 to 10000 (valid for all comonomers),
carboxylates based on maleic acid copolymers, in particular derived from maleic anhydride copolymers, wherein
the copolymers can have a blockwise or random distribution of the comonomer units, for example

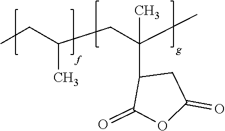

with
f=2 to 10000, preferred 10 to 10000, more preferred 100 to 10000, even more preferred 1000 to 10000 (valid for all comonomers), and
g=2 to 10000, preferred 10 to 10000, more preferred 100 to 10000, even more preferred 1000 to 10000,
carboxylates based on poly (itaconic acid) homo und copolymers, i.e. derived from poly itaconic acid homopolymers

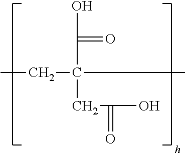

with h=2 to 10000, preferred 10 to 10000, more preferred 100 to 10000, even more preferred 1000 to 10000,
or from poly itaconic acid copolymers,
i.e. containing non-reactive comonomers, wherein
the copolymers can have a blockwise or random distribution of the comonomer units, for example

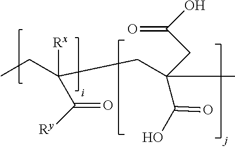

R^{x}=CH₃, R^{y}=OCH₃ Poly(methylmethacrylate-co-itaconic acid—PMIAA
R^{x}=H, R^{y}=NH₂ Poly(acrylamide-co-itaconic acid) PAIAA
   with
   i=2 to 10000, preferred 10 to 10000, more preferred 100 to 10000, even more preferred 1000 to 10000 (valid for all comonomers),
   j=2 to 10000, preferred 10 to 10000, more preferred 100 to 10000, even more preferred 1000 to 10000,
   or from poly itaconic acid copolymers containing comonomers providing OH and amine functions, which can be functionalised via additional ester or amide bonds, in particular with fatty acids or poly fatty acids, for example 2-hydroxyethyl methacrylate-itaconic acid copolymers,
   or from poly itaconic acid copolymers containing carboxylic acid functions containing comonomers, wherein
   the copolymers can have a blockwise or random distribution of the comonomer units, for example

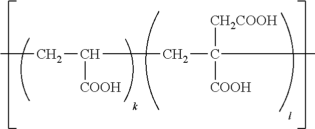

with
k=2 to 10000, preferred 10 to 10000, more preferred 100 to 10000, even more preferred 1000 to 10000 (valid for all comonomers),
l=2 to 10000, preferred 10 to 10000, more preferred 100 to 10000, even more preferred 1000 to 10000,
wherein the anions of this group are preferably di- to 30000-valent, more preferably kiliavalent, even more preferably deca- to kiliavalent, even further preferably pentaconta- to kiliavalent, and most preferably hecta- to kiliavalent anions.

According to the invention, any cationic structure according to the invention can be combined with any anion according to the invention.

It is preferable to combine cations comprising a low number of quaternary nitrogen atoms, i.e. 1 to 20, in particular 1 to 10, more particular 1 to 6 and even more particular 1 or 2 quaternary nitrogen atoms, with mono- to deca-valent anions, preferably mono- to hexavalent anions, more preferably mono- to trivalent anions, even more preferably monovalent anions or divalent anions.

Alternatively, undeca- to 30000-valent polyanions, in particular undeca- to hectavalent polyanions or henhectavalent to kiliavalent polyanions can be used.

Polyquat cations comprising 21 or more quaternary nitrogen atoms are typically combined with lower valent counter ions, i.e. mono- to pentacontavalent anions, more preferably mono- to decavalent anions, even more preferably mono- to pentavalent anions, and most preferably mono- and divalent counter anions, in particular chloride anions, monocarboxylate and dicarboxylate anions. Herein, the usage of anions being more than 50-valent is less preferred.

As used above, R⁷ is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, tertiary amino groups

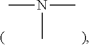

quaternary ammonium groups

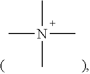

and which can be substituted with OH groups or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group.

According to the invention, the radicals R⁷ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzylor tolyl, in particular from such groups having 6 to 24 carbon atoms, each optionally containing one or more functional groups as indicated above.

More preferably, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom.

There is no limitation regarding at which C-atoms of the hydrocarbyl radicals the adjacent groups C(O) group or X group are bonded to R⁷.

However, R⁷ is preferably derived from a carboxylic acid or a hydroxycarboxylic acid bearing one or more hydroxylic groups, more preferably from a carboxylic acid or monohydroxy carboxylic acid, most preferably from C7-C25 fatty acid bearing no hydroxyl group as substituent. Accordingly, R⁷ preferably represents the alkyl or alkenyl chain of such carboxylic acids. For instance, if R⁷ is derived from ricinoleic acid

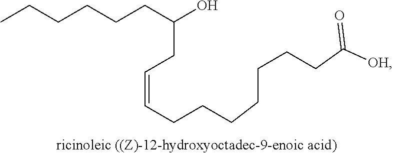 [SMILES: CCCCCCC(O)C/C=C\CCCCCCCC(=O)O]

then R⁷ represents an 11-hydroxy heptadec-8-enyl radical

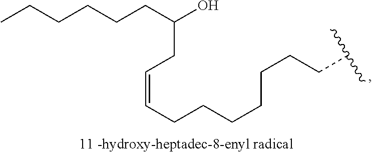

or if R⁷ is derived from oleic acid,

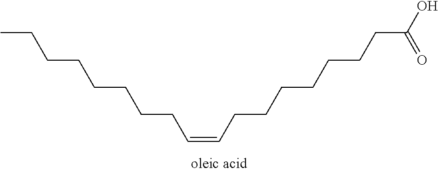 [SMILES: CCCCCCCC/C=C\CCCCCCCC(=O)O]

then R⁷ represents a heptadec-8-enyl radical

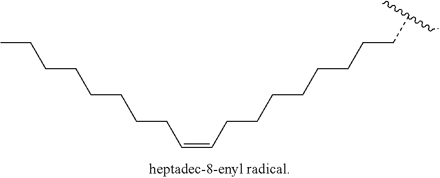

Preferred examples for R⁷ are the structures derived from a corresponding carboxylic acid or hydroxyl carboxylic acid by abstraction of the carboxylate group, wherein the carboxylic acid may be selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid.

Although the radical R⁷ can optionally contain one or more groups selected from —O—, —NH—, —C(O)—, —C(S)—, tertiary amino groups

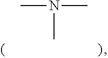

quaternary ammonium groups

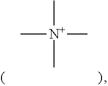

and may be substituted with OH groups or halide groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

Considering the structures of the formulas (I) and (II) and the definitions of R¹, R², R³, R⁴ and R⁵, it is clear that in some cases the substructures of a given compound according to the invention may be assigned to the residues R¹, R², R³, R⁴ and R⁵ displayed in the formulas (I) and (II) and defined above in more than one way.

Only in such case, the following rules are to be applied for the assignment of the substructures to said terms R¹-R⁵ in a clear way in this order:
R¹ is to be selected in such manner that the subscript x, i.e. the number of groups —(—F) bonded to R¹, is as high as possible;
R¹ is to be selected in such manner that the number of groups F with n≠0 is as low as possible;
If there are several options to fulfill the foregoing requirements, R¹ is to be selected in such manner that the number of carbon atoms in R¹ is as high as possible;
If there are still two or more substructures fulfilling the foregoing requirements in the same way, R¹ is to be selected in such manner that it is the substructure having the highest sum of atomic weight of the atoms contained in the substructure among the substructures possible.

It is noted that according to the invention, if several of any of the residues R², R³, R⁴, R⁵, R⁶ or R⁷ are present in a compound according to the invention, each of the residues can represent another substructure as defined above, i.e. each R², R³, R⁴, R⁶ and R⁷ group is independently selected according to the definitions according to the invention.

In a preferred embodiment of the present invention, a compound of the formula:

R¹(—F)ₓ (I)

as defined above is provided,
wherein x is 2 to 50.

More preferably, according to this embodiment x is in the range of 3 to 50, 4 to 50, 5 to 50, 6 to 50, 7to 50, 8 to 50, 9 to 50, 10to 50, 2 to 40, 2 to 35, 2 to 30, 2 to 25, 2 to 20, 2 to 15 or 2 to 10.

In another preferred embodiment according to the invention, the compound of the general formula

R¹(—F)ₓ (1)

as defined above does not comprise a poly(ethylene oxide) or poly(propylene oxide) unit.

According to the invention, a poly(ethylene oxide) unit is defined as a unit represented by the formula (CH₂CH₂O)ₓ with x≥2, and a or poly(propylene oxide) unit is defined as a unit represented by the formula (CH₂CH(CH₃)O]ₓ with x≥2.

In a further preferred embodiment according to the invention, in the compound of the general formula

R¹(—F)ₓ (I)

as defined above R¹ contains at least one moiety of the general formula (IIIa)

(—X—C(O)—R⁶)ₘ—X—C(O)—R⁷ (IIIa),

or of the general formula (IVa)

(—C(O)—X—R⁶)ₘ—C(O)—X—R⁷ (IVa)

wherein X and R⁶ and m are as defined above, and
R⁷ is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, tertiary amino groups

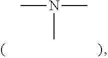

quaternary ammonium groups

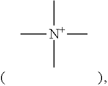

and which can be substituted with OH groups or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group.

According to this embodiment, the radicals R⁷ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzylor tolyl, in particular from such groups having 6 to 24 carbon atoms, each optionally containing one or more functional groups as indicated above.

More preferred, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent X group by a terminal C-atom.

There is no limitation regarding at which C-atoms of the hydrocarbyl radicals the adjacent groups X group is bonded to R⁷.

However, R⁷ is preferably derived from a carboxylic acid or a hydroxycarboxylic acid bearing one or more hydroxylic groups, more preferably from a carboxylic acid or monohydroxy carboxylic acid, most preferably from C7-C25 fatty acid bearing no hydroxyl group as substituent. Accordingly, R⁷ preferably represents the alkyl or alkenyl chain of such carboxylic acids, as is illustrated in the above-given example for the group R⁷.

Preferred examples for R⁷ according to this embodiment are the structures derived from a corresponding carboxylic acid or hydroxyl carboxylic acid by abstraction of the carboxylate group, wherein the carboxylic acid may be selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, oleic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid.

More preferred, the R⁷ radicals according to this embodiment are derived from palmitic acid, margaric acid, stearic acid, linoleic acid, α-linolenic acid, γ-linolenic acid, oleic acid, nonadecylic acid, arachidic acid, 10-hydroxy stearic acid, 12-hydroxy stearic acid, ricinoleic acid, lesquerolic acid or from 2,2′-di-hydroxymethyl propanoic acid Most preferred R⁷ radicals according to this embodiment are oleic acid, stearic acid, lesquerolic acid and ricinoleic acid.

Although the radical R⁷ can optionally contain one or more groups selected from —O—, —NH—, —C(O)—, —C(S)—, tertiary amino groups

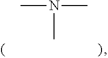

quaternary ammonium groups

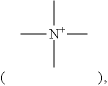

and may be substituted with OH groups or halide groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

In yet a further preferred embodiment according to the invention, in the compound of the general formula

R¹(—F)ₓ (I)

as defined above only one or more of the residues R¹ or R² contain at least one moiety of the general formulas (III) or (IV)

(—X—C(O)—R⁶)ₘ—X—C(O)— (III) or

(—C(O)—X—R⁶)ₘ—C(O)—X— (IV),

preferably only one or more of the residues R¹ or R² contain at least one moiety of the general formula (IIIa) or of the general formula (IVa)

(—X—C(O)—R⁶)ₘ—X—C(O)R⁷ (IIIa),

(—C(O)—X—R⁶)ₘ—C(O)—X—R⁷ (IVa),

wherein X, R⁶, R⁷ and m are as defined above.

According to this embodiment, it is preferred when in the moieties of the formula (IIIa) or (Iva) of the residues R¹ or R²

### X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene,
R⁷ is selected from hexadecyl, heptadecyl, octadecyl, nonadecyl, eicosyl, hexadecenyl, heptadecenyl, octadecenyl, nonadecenyl, eicosenyl,
and m is 1, 2, 3, 4 or 5.

It is most preferred according to this embodiment when

### X=O,

R⁶ is derived from ricinoleic acid, 10-hydroxy stearic acid, 12-hydroxy stearic acid or lesquerolic acid,
R⁷ is derived from oleic acid, ricinoleic acid or stearic acid, and m is 1, 2, 3, 4 or 5.

In another preferred embodiment according to the invention, in the compound of the formula R¹(—F)ₓ (I) as defined above at least 1% of all groups F contain at least one moiety of the general formula (III) or (IV), more preferably at least 10% of all groups F contain at least one moiety of the general formula (III) or (IV), even more preferably at least 50% of all groups F contain at least one moiety of the general formula (III) or (IV), and most preferably 100% of all groups F contain at least one moiety of the general formulas (III) or (IV), or wherein at least 1% of all groups F contain at least one moiety of the general formula (IIIa) or (IVa), more preferably at least 10% of all groups F contain at least one moiety of the general formula (IIIa) or (IVa), even more preferably at least 50% of all groups F contain at least one moiety of the general formula (IIIa) or (IVa), and most preferably 100% of all groups F contain at least one moiety of the general formulas (IIIa) or (IVa).

According to this embodiment, it is preferred when each moiety of the general formula (III) or (IV) of the groups F contains at least one R⁶ selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene,

more preferred each moiety of the general formula (III) or (IV) of the groups F contains at least one R⁶ selected optionally hydroxyl-substituted from hexadecylene, heptadecylene, octadecylene, nonadecylene, eicosylene, hexadecenylene, heptadecenylene, octadecenylene, nonadecenylene, eicosenylene,
and m is 1, 2, 3, 4 or 5, and most preferably R⁶ in each moiety of the general formula (III) or (IV) of the groups F is derived from ricinoleic acid, 10-hydroxy stearic acid, 12-hydroxy stearic acid or lesquerolic acid,
and m is 1, 2, 3, 4 or 5.

In a further preferred embodiment according to the invention, in the compound of the formula R¹(—F)ₓ (I) as defined above at least 1% of all groups R² contain at least one moiety of the general formula (III) or (IV), more preferably at least 10% of all groups R² contain at least one moiety of the general formula (III) or (IV), even more preferably at least 50% of all groups R² contain at least one moiety of the general formula (III) or (IV), and most preferably 100% of all groups R² contain at least one moiety of the general formulas (III) or (IV), or wherein at least 1% of all groups R² contain at least one moiety of the general formula (IIIa) or (IVa), more preferably at least 10% of all groups R² contain at least one moiety of the general formula (IIIa) or (IVa), even more preferably at least 50% of all groups R² contain at least one moiety of the general formula (IIIa) or (IVa), and most preferably 100% of all groups R² contain at least one moiety of the general formulas (IIIa) or (IVa).

According to this embodiment, it is preferred when each moiety of the general formula (III) or (IV) of the groups R² contains at least one R⁶ selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene,

more preferred when each moiety of the general formula (III) or (IV) of the groups R² contains at least one R⁶ selected optionally hydroxyl-substituted from hexadecylene, heptadecylene, octadecylene, nonadecylene, eicosylene, hexadecenylene, heptadecenylene, octadecenylene, nonadecenylene, eicosenylene,
and m is 1, 2, 3, 4 or 5, and
most preferably R⁶ in each moiety of the general formula (III) or (IV) of the groups R² is derived from ricinoleic acid, 10-hydroxy stearic acid, 12-hydroxy stearic acid or lesquerolic acid, and m is 1, 2, 3, 4 or 5.

In another preferred embodiment according to the invention, in the compound of the formula R¹(—F)ₓ (I) as defined above at least 1% of all groups R³, R⁴ and R⁵ contain at least one moiety of the general formula (III) or (IV), more preferably at least 10% of all groups R³, R⁴ and R⁵ contain at least one moiety of the general formula (III) or (IV), even more preferably at least 50% of all groups R³, R⁴ and R⁵ contain at least one moiety of the general formula (III) or (IV), and most preferably 100% of all groups R³, R⁴ and R⁵ contain at least one moiety of the general formulas (III) or (IV), or wherein at least 1% of all groups R³, R⁴ and R⁵ contain at least one moiety of the general formula (IIIa) or (IVa), more preferably at least 10% of all groups R³, R⁴ and R⁵ contain at least one moiety of the general formula (IIIa) or (IVa), even more preferably at least 50% of all groups R³, R⁴ and R⁵ contain at least one moiety of the general formula (IIIa) or (IVa), and most preferably 100% of all groups R³, R⁴ and R⁵ contain at least one moiety of the general formulas (IIIa) or (IVa).

According to this embodiment, it is preferred when each moiety of the general formula (III) or (IV) of the groups R³, R⁴ and R⁵ contains at least one R⁶ selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene,

more preferred when each moiety of the general formula (III) or (IV) of the groups R³, R⁴ and R⁵ contains at least one R⁶ selected optionally hydroxyl-substituted from hexadecylene, heptadecylene, octadecylene, nonadecylene, eicosylene, hexadecenylene, heptadecenylene, octadecenylene, nonadecenylene, eicosenylene,
and m is 1, 2, 3, 4 or 5, and
most preferably R⁶ in each moiety of the general formula (III) or (IV) of the groups R³, R⁴ and R⁵ is derived from ricinoleic acid, 10-hydroxy stearic acid, 12-hydroxy stearic acid or lesquerolic acid,
and m is 1, 2, 3, 4 or 5.

In still another preferred embodiment according to the invention, in the compound of the general formula R¹(—F)ₓ (I) as defined above

x is 2 and the compound is of the general formula (V):

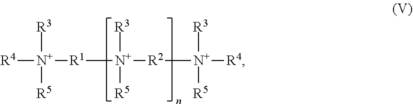

wherein R¹, R², R³, R⁴, R⁵ and n are as defined above.

In a preferred embodiment of the present invention, a compound of the formula:

R¹(—F)ₓ (I)

as defined above is provided, wherein
R¹ is selected from monovalent to pentacontavalent, optionally substituted hydrocarbon radicals which have up to 1000 carbon atoms, preferred 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, tertiary amino groups

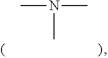

groups and can be substituted by —OH groups and halide groups, preferably R¹ is a C3-C18 glycerol-based polyether radical or a C1-C8 linear alkylene radical, and
F has the general formula (VI), which corresponds to formula (II) with n being equal to 0:

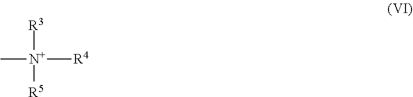

and the groups F bind to a carbon atom of R¹,
wherein
R³, R⁴, R⁵ are independently selected from hydrogen and optionally substituted straight-chain, cyclic or branched, saturated, unsaturated or aromatic hydrocarbon radicals which have up to 300 carbon atoms, preferred 1 to 200 carbon atoms, more preferred 1 to 150 carbon atoms, even more preferred 1 to 50 carbon atoms, specifically 1 to 20 carbon atoms, more specifically 1 to 10 carbon atoms which optionally contain one or more groups selected from —O—, —NH—, —C(O)—, —C(S)—, tertiary amino groups

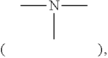

quaternary ammonium groups

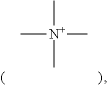

and can be substituted by OH, preferably R³ to R⁵ are C1-C8 linear alkyl groups, such as methyl ethyl, propyl or butyl, or linear alkyl groups containing one or more moieties of the general formula (III) or (IV), more preferably linear alkyl groups terminated by a group of the general formula (IIIa) or (IVa),
the counter ions A⁻ are selected from mono- to trivalent inorganic anions and mono- to 30000-valent, preferably mono- to kiliavalent organic anions, preferably selected from halide anions, such as chloride, bromide, iodide, sulphate, phosphate, phosphonate, sulphonate, methosulphate, carboxylate anions, such as acetate, propionate, lactate, octanoate, 2-ethyl-hexanoate, dodecanoate, hexadecanoate, octadecanoate, oleate, ricinololate, 12-hydroxy-octadecanoate, succinate, maleate, tartrate, polyethercarboxylate, polymeric fatty acid carboxylates of the type
R¹[(—C(O)—X—R⁶)ₘ—C(O)—X—R⁷]ₓ or
R¹[(X—C(O)—R⁶)ₘ—X—C(O)—R⁷]ₓ, wherein either R¹ or at least one of R⁷, or both R¹ and at least one of R⁷ bear one or more carboxylate groups,
preferably with X=O,
in particular
linear polymeric fatty acid carboxylates of the type
   ⁻O—C(O)—R⁶(—X—C(O)—R⁶)ₘ₋₁—X—C(O)—R⁷, preferably
   ⁻O—C(O)—R⁶—(O—C(O)—R⁶)ₘ—O—C(O)—R⁷,
   i.e. derived from linear poly fatty acid structures,
branched linear polymeric fatty acid carboxylates,
   i.e. derived from branched poly fatty acid structures,
   in particular branched linear polymeric fatty acid carboxylates derived from partial esters of polyfunctional carboxylic acids, in particular of the dicarboxylic acids succinic acid and maleic acid, with castor oil or lesquerella oil, such as

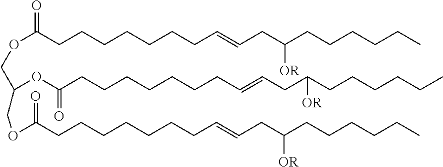

with one

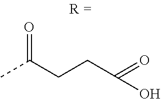

and the remaining two

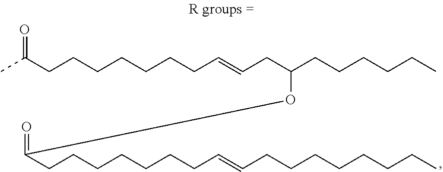

dendritic polymeric fatty acid carboxylates,
   i.e. derived from dendritic poly fatty acid structures,
   or of the types

X—R⁶(—C(O)—X—R⁶)ₘ₋₁—C(O)—X—R⁷ or

R⁶(—C(O)—X—R⁶)ₘ₋₁—C(O)—X—R⁷,

wherein in the two latter types the R⁷ group bears at least one anionic carboxylate group, or of the type

R¹[(—C(O)—X—R₆)ₘ—C(O)O⁻]ₓ,

and wherein X, R¹, R⁶, R⁷, m and x are as defined above
wherein the counter ions A⁻ of this group are preferably mono- to pentacontavalent, more preferably mono- to decavalent, even more preferably mono- to pentavalent, most preferably pentavalent, tetravalent, trivalent, divalent or monovalent anions,
or the counter anions are selected from the group consisting of carboxylate anions based on poly (acrylic acid) homo- and co-polymers,
i.e. carboxylates derived from poly acrylic acid homopolymers

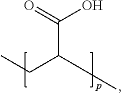

wherein p=2 to 10000, preferred 10 to 10000, more preferred 100 to 10000, even more preferred 1000 to 10000,
carboxylate anions derived from poly acrylic acid copolymers,
i.e. containing non-reactive comonomers, for example

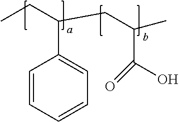

with
a=2 to 10000, preferred 10 to 10000, more preferred 100 to 10000, even more preferred 1000 to 10000
b=2 to 10000, preferred 10 to 10000, more preferred 100 to 10000, even more preferred 1000 to 10000 (valid for all comonomers), wherein
the copolymers can have a blockwise or random distribution of the comonomer units,
or carboxylate anions derived from poly acrylic acid copolymers
containing comonomers providing OH and amine functions, which can be functionalised via additional ester or amide bonds, in particular with fatty acids or poly fatty acids, for example

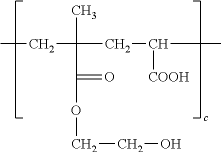

with c=2 to 10000, preferred 10 to 10000, more preferred 100 to 10000, even more preferred 1000 to 10000,
or carboxylate anions derived from poly acrylic acid copolymers containing carboxylic acid functions-containing comonomers, wherein
the copolymers can have a blockwise or random distribution of the comonomer units, for example

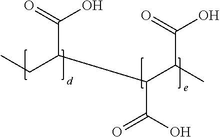

with
d=2 to 10000, preferred 10 to 10000, more preferred 100 to 10000, even more preferred 1000 to 10000
e=2 to 10000, preferred 10 to 10000, more preferred 100 to 10000, even more preferred 1000 to 10000 (valid for all comonomers),
carboxylates based on maleic acid copolymers, in particular derived from maleic anhydride copolymers, wherein
the copolymers can have a blockwise or random distribution of the comonomer units, for example

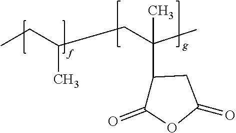

with
f=2 to 10000, preferred 10 to 10000, more preferred 100 to 10000, even more preferred 1000 to 10000 (valid for all comonomers), and
g=2 to 10000, preferred 10 to 10000, more preferred 100 to 10000, even more preferred 1000 to 10000,
carboxylates based on poly (itaconic acid) homo und copolymers,
i.e. derived from poly itaconic acid homopolymers

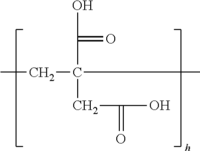

with h=2 to 10000, preferred 10 to 10000, more preferred 100 to 10000, even more preferred 1000 to 10000,
or from poly (itaconic acid) copolymers,
i.e. containing non-reactive comonomers, wherein
the copolymers can have a blockwise or random distribution of the comonomer units, for example

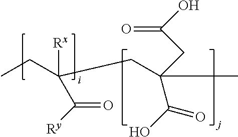

R^{x}=CH₃, R^{y}=OCH₃ Poly(methylmethacrylate-co-itaconic acid-PMIAA
R^{x}=H, R^{y}=NH₂ Poly(acrylamide-co-itaconic acid) PAIAA
   with
   i=2 to 10000, preferred 10 to 10000, more preferred 100 to 10000, even more preferred 1000 to 10000 (valid for all comonomers),
   j=2 to 10000, preferred 10 to 10000, more preferred 100 to 10000, even more preferred 1000 to 10000,
   or from poly itaconic acid copolymers containing comonomers providing OH and amine functions, which can be functionalised via additional ester or amide bonds, in particular with fatty acids or poly fatty acids, for example 2-hydroxyethyl methacrylate—itaconic acid copolymers,
   or from poly itaconic acid copolymers containing carboxylic acid functions containing comonomers, wherein
   the copolymers can have a blockwise or random distribution of the comonomer units, for example

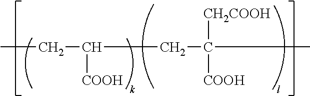

with
k=2 to 10000, preferred 10 to 10000, more preferred 100 to 10000, even more preferred 1000 to 10000 (valid for all comonomers),
l=2 to 10000, preferred 10 to 10000, more preferred 100 to 10000, even more preferred 1000 to 10000,
wherein the anions of this group are preferably di- to 30000-valent, more preferably di- to kiliavalent, even more preferably deca- to kiliavalent, even further preferably pentaconta- to kiliavalent, and most preferably hecta- to kiliavalent anions,
with the proviso that at least one of the radicals R¹, R³, R⁴, R⁵ of the cationic structures of the general formulas (I) and (II) contains at least one moiety of the general formulas (IIIa) or (IVa):

(—X—C(O)—R⁶)ₘ—X—C(O)—R⁷ (IIIa)

(—C(O)—X—R⁶)ₘ—C(O)—X—R⁷ (IVa)

wherein X is as defined above,
m=1 to 20, preferred 1 to 10, more preferred 1 to 6, even more preferred 2 to 6, specifically 1, 2, 3, 4, 5, 6, and
R¹¹ is preferably selected from the group consisting of hydrogen, n-, iso-, or tert. —C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, C₆-C₃₀-alkylaryl, C₂-C₂₂-alkenyl, C₂-C₂₂-alkenyloxyalkyl, which optionally can be each substituted by hydroxyl and halogen, and which optionally can contain one or more ether groups (—O—), preferably hydrogen or n-, iso-, or tert. —C₁-C₂₂-alkyl,
R⁶ is independently selected from optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms, preferably R⁶ is a C6 to C24 linear alkylene or alkenylene group, most preferably derived from ricinoleic acid or lesquerolic acid,
R⁷ is independently selected from optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms, optionally containing one or more groups selected from —O—, —NH—, —C(O)—, —C(S)—, tertiary amino groups

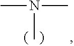

quaternary ammonium groups

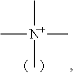

and which can be substituted with OH groups or halide groups, wherein the radical R⁷ cannot contain a combination of a —C(O)— group and a —O— group or a combination of a —C(O)— group and a —NH— or tertiary amino group forming an internal carboxylate group or an internal amide group., preferably R⁷ is a C6 to C24 alkyl or alkenyl group, more preferably a linear C12 to C24 alkyl or C12 to C24 alkenyl group, most preferably derived from linoleic, linolenic or oleic acid, with the proviso that at least one R⁶ has more than 6 carbon atoms, and that for x=1
R¹, R³, R⁴, R⁵ do not bind through —OCH₂CH₂— to the nitrogen atom of the group

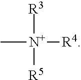

In a further preferred embodiment of the present invention, a compound of the formula:

R¹(—F)ₓ (I) is provided, wherein

R¹ is selected from monovalent to pentacontavalent, preferred monovalent to triacontavalent, more preferred monovalent to eicosavalent, even more preferred monovalent to decavalent, specifically monovalent, divalent trivalent, tetravalent, pentavalent, hexavalent, heptavalent, octavalent, nonavalent, decavalent optionally substituted hydrocarbon radicals which have up to 1000 carbon atoms, preferred 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, tertiary amino groups

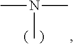

quaternary ammonium groups

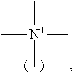

and can be substituted by —OH, and
F has the general formula (VI):

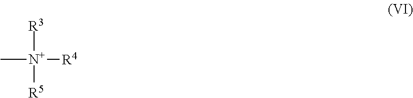

and the groups F bind to a carbon atom of R¹, wherein
R³, R⁴, R⁵ are selected from optionally substituted straight-chain, cyclic or branched, saturated, unsaturated or aromatic hydrocarbon radicals which have up to 300 carbon atoms, preferred 1 to 200 carbon atoms, more preferred 1 to 150 carbon atoms, even more preferred 1 to 50 carbon atoms, specifically 1 to 20 carbon atoms, more specifically 1 to 10 carbon atoms which optionally contain one or more groups selected from —O—, —NH—, —C(O)—, —C(S)—, tertiary amino groups

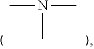

quaternary ammonium groups

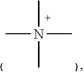

and can be substituted by OH,
the counter ions A⁻ are selected from mono- to trivalent inorganic anions and mono- to 30000-valent, in particular mono- to kiliavalent organic anions, preferably selected from halide anions, such as chloride, bromide, iodide, sulphate, phosphate, phosphonate, sulphonate, methosulphate, carboxylate anions, such as acetate, propionate, lactate, octanoate, 2-ethyl-hexanoate, dodecanoate, hexadecanoate, octadecanoate, oleate, ricinololate, 12-hydroxy-octadecanoate, succinate, maleate, tartrate, polyethercarboxylate, polymeric fatty acid carboxylates of the type
R¹[(—C(O)—X—R⁶)ₘ—C(O)—X—R⁷]ₓ or
R¹[(X—C(O)—R⁶)ₘ—X—C(O)—R⁷]ₓ, wherein either R¹ or at least one of R⁷, or both R¹ and at least one of R⁷ bear one or more carboxylate groups,
preferably with X=O,
in particular
linear polymeric fatty acid carboxylates of the type

—O—C(O)—R⁶(—X—C(O)—R⁶)ₘ₋₁—X—C(O)—R⁷, preferably

—O—C(O)—R⁶—(O—C(O)—R⁶)ₘ—O—C(O)—R⁷,

i.e. derived from linear poly fatty acid structures,
branched linear polymeric fatty acid carboxylates,
   i.e. derived from branched poly fatty acid structures, in particular branched linear polymeric fatty acid carboxylates derived from partial esters of polyfunctional carboxylic acids, in particular of the dicarboxylic acids succinic acid and maleic acid, with castor oil or lesquerella oil, such as

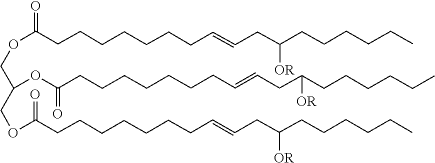

with on

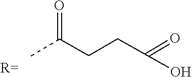

and the remaining two

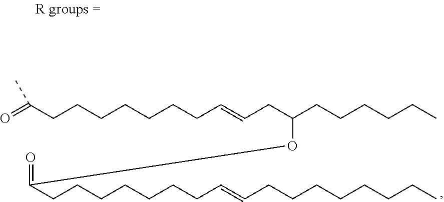

dendritic polymeric fatty acid carboxylates,
   i.e. derived from dendritic poly fatty acid structures,
   or of the types

X—R⁶(—C(O)—X—R⁶)ₘ₋₁—C(O)—X—R⁷ or

R⁶(—C(O)—X—R⁶)ₘ₋₁—C(O)—X—R⁷,

wherein in the two latter types the R⁷ group bears at least one anionic carboxylate group, or of the type

R¹[(—C(O)—X—R₆)ₘ—C(O)O⁻]ₓ,

and wherein X, R¹, R⁶, R⁷, m and x are as defined above and
wherein the counter ions A⁻ of this group are preferably mono- to pentacontavalent, more preferably mono- to decavalent, even more preferably mono- to pentavalent, most preferably pentavalent, tetravalent, trivalent, divalent or monovalent anions,
or the counter ions are selected from the group consisting of carboxylate anions based on poly (acrylic acid) homo- and co-polymers,
i.e. carboxylates derived from poly acrylic acid homopolymers

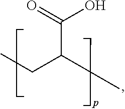

wherein p=2 to 10000, preferred 10 to 10000, more preferred 100 to 10000, even more preferred 1000 to 10000,
carboxylate anions derived from poly acrylic acid copolymers,
i.e. containing non-reactive comonomers, for example

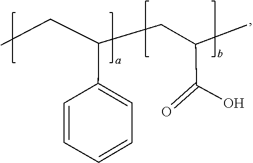

with
a=2 to 10000, preferred 10 to 10000, more preferred 100 to 10000, even more preferred 1000 to 10000
b=2 to 10000, preferred 10 to 10000, more preferred 100 to 10000, even more preferred 1000 to 10000 (valid for all comonomers), wherein
the copolymers can have a blockwise or random distribution of the comonomer units, or carboxylate anions derived from poly acrylic acid copolymers
containing comonomers providing OH and amine functions, which can be functionalised via additional ester or amide bonds, in particular with fatty acids or poly fatty acids, for example

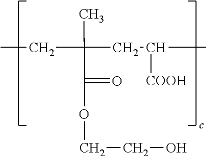

with c=2 to 10000, preferred 10 to 10000, more preferred 100 to 10000, even more preferred 1000 to 10000,
or carboxylate anions derived from poly acrylic acid copolymers containing carboxylic acid functions-containing comonomers, wherein
the copolymers can have a blockwise or random distribution of the comonomer units, for example

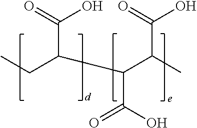

with
d=2 to 10000, preferred 10 to 10000, more preferred 100 to 10000, even more preferred 1000 to 10000
e=2 to 10000, preferred 10 to 10000, more preferred 100 to 10000, even more preferred 1000 to 10000 (valid for all comonomers),
carboxylates based on maleic acid copolymers, in particular derived from maleic anhydride copolymers, wherein
the copolymers can have a blockwise or random distribution of the comonomer units, for example

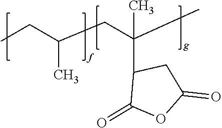

with
f=2 to 10000, preferred 10 to 10000, more preferred 100 to 10000, even more preferred 1000 to 10000 (valid for all comonomers), and
g=2 to 10000, preferred 10 to 10000, more preferred 100 to 10000, even more preferred 1000 to 10000,
carboxylates based on poly (itaconic acid) homo und copolymers,
i.e. derived from poly itaconic acid homopolymers

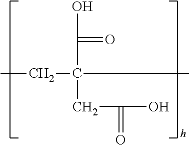

with h=2 to 10000, preferred 10 to 10000, more preferred 100 to 10000, even more preferred 1000 to 10000,
or from poly itaconic acid copolymers,
i.e. containing non-reactive comonomers, wherein
the copolymers can have a blockwise or random distribution of the comonomer units, for example

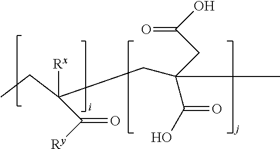

R^{x}=CH₃, R^{y}=OCH₃ Poly(methylmethacrylate-co-itaconic acid-PMIAA
R^{x}=H, R^{y}=NH₂ Poly(acrylamide-co-itaconic acid) PAIAA
   with
   i=2 to 10000, preferred 10 to 10000, more preferred 100 to 10000, even more preferred 1000 to 10000 (valid for all comonomers),
   j=2 to 10000, preferred 10 to 10000, more preferred 100 to 10000, even more preferred 1000 to 10000,
   or from poly itaconic acid copolymers containing comonomers providing OH and amine functions, which can be functionalised via additional ester or amide bonds, in particular with fatty acids or poly fatty acids, for example 2-hydroxyethyl methacrylate-itaconic acid copolymers,
   or from poly itaconic acid copolymers containing carboxylic acid functions containing comonomers, wherein
   the copolymers can have a blockwise or random distribution of the comonomer units, for example

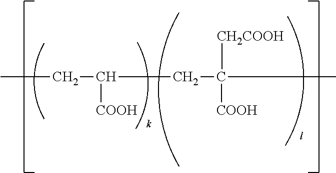

with
k=2 to 10000, preferred 10 to 10000, more preferred 100 to 10000, even more preferred 1000 to 10000 (valid for all comonomers),
l=2 to 10000, preferred 10 to 10000, more preferred 100 to 10000, even more preferred 1000 to 10000,
wherein the anions of this group are preferably di- to 30000-valent, more preferably di- to kiliavalent, even more preferably deca- to kiliavalent, and most preferably hecta- to kiliavalent anions,
with the proviso that at least one of the radicals R¹, R³, R⁴, R⁵ of the cationic structure of the formulas (I) and (II) contains at least one moiety of the general formulas (VII) or (Vill):

—X—C(O)—R^{x}—(X—C(O)—R^{x})ₘ—X—C(O)—R⁷ (VII) or

—X—C(O)—R^{x}—(X—C(O)—R^{x})ₘ—X—C(O)—R⁷ (VIII)

wherein

### X is 0 or NR,

m=1 to 20, preferred 1 to 10, more preferred 1 to 6, even more preferred 2 to 6, specifically 1, 2, 3, 4, 5, 6 and
the total number of carbon atoms in R^{x}+R⁷ (Σcarbon atoms R^{x}, R⁷) is 19 to 300, preferred 25 to 300, more preferred 35 to 300, even more preferred 50 to 300, specifically 35 to 200, more specifically 35 to 150, even more specifically 50 to 150,
R¹¹ is preferably selected from the group consisting of hydrogen, n-, iso-, or tert. —C₁-C₂₂-alkyl, more preferred hydrogen,
R^{x} is optionally OH, —O—C(O)—R⁷, —O—C(O)—R⁶—(O—C(O)—R⁶)₀₋₁₉—O—C(O)—R⁷ substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms, preferably derived from monohydroxy carboxylic acids, in particular glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, lesquerolic acid, ricinoleic acid, or dihydroxy carboxylic acids, in particular 2,2′-di-hydroxymethyl propanoic acid, 9,10-dihydroxy stearic acid, or polyhydroxy carboxylic acids, in particular gluconic acid,
R⁶ is as defined above,
R⁷ is optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms, preferably derived from acetic acid, octanoic acid, nonanoic acid, decanoic acid, dodecanoic acid, tetradecanoic acid, hexadecanoic acid, octadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, erucic acid.

According to this embodiment, it is preferred when R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, more preferably independently selected from optionally hydroxyl-substituted hexadecylene, heptadecylene, octadecylene, nonadecylene, eicosylene, hexadecenylene, heptadecenylene, octadecenylene, nonadecenylene, eicosenylene, most preferably each R⁶ is independently derived from ricinoleic acid, 10-hydroxy stearic acid, 12-hydroxy stearic acid or lesquerolic acid.

In a further preferred embodiment of the present invention, a compound of the formula:

R¹(—F)ₓ (I)

as defined above is provided, wherein
R¹ is selected from the group consisting of:
monovalent to octadecavalent, preferably divalent to octadecavalent, more preferably divalent to hexavalent, even more preferably divalent, trivalent and tetravalent optionally OH or amido substituted straight-chain, cyclic or branched, saturated, unsaturated or aromatic hydrocarbyl groups, derived from tertiary amines having at least three, preferred more than three carbon atoms, in particular trimethylamine, triethylamine, tributylamine, N,N-dimethylethanolamine, N,N-dimethylpropanolamine, N-methyl imidazole, N,N,N′,N′-tetramethyl-1,2-diaminoethane, N,N,N′,N′-tetramethyl-1,4-diaminobutane, N,N,N′,N′-tetramethyl-1,6-diaminohexane, N,N,N′,N′,N′-pentamethyl-diethylenetriamine, N,N,N′,N′,N′-pentamethyl-dipropylenetriamine, bis-(2-dimethylaminoethyl)ether, bis-(2-dimethylaminopropyl)ether, 2,2′-dimorpholinodiethylether, N,N-bis-(3-dimethylaminopropyl)-N-isopropanolamine, N,N,N′-trimethylaminoethyl-ethanolamine, 1,3,5-tris(3-(dimethylamino)propyl)-hexahydro-s-triazine, condensation products of epoxy compounds, in particular glycidyl ethers, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol etc., or derived from polypropylene glycols, like dipropylene glycol (e.g, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid, with primary or secondary amino functionalized amines, in particular N,N-dimethylpropylenediamine, N,N,N′,N′-tetramethyl-diethylenetriamine, N,N,N′,N′-tetramethyl-dipropylenetriamine, N-methylmorpholine, N-methylpiperazine; and
monovalent to octadecavalent, preferably divalent to octadecavalent, more preferably divalent to hexavalent, even more preferably divalent, trivalent and tetravalent optionally OH, amino or amido substituted straight-chain, cyclic or branched, saturated, unsaturated or aromatic hydrocarbon groups, derived from alkyl halogenides having more than one, preferred more than two carbon atoms such as alkyl chlorides, bromides, iodides, e.g. 1,3-dichloropropane, 1,3-dichlorobutane, 1,4-dichlorobutane, dichloro-monohydroxy propane isomers, 1,2,3-trichloro propane, 1,2-dichloro hexanediol, 1,2-dichloro hexane, or the respective bromides and iodide derivatives;
monovalent to octadecavalent, preferably divalent to octadecavalent, more preferably divalent to hexavalent, even more preferably divalent, trivalent and tetravalent optionally OH, amino or amido substituted straight-chain, cyclic or branched, saturated, unsaturated or aromatic hydrocarbon groups, derived from esters of halogenated carboxylic acids, preferred, chloro carboxylic acids, in total (ester) having more than two, preferred more than three carbon atoms such as esters of chloroacetic acid, 3-chloropropionic acid, 4-chlorobutanoic acid or the respective bromo carboxylic acids, with alcohols, in particular methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol, or derived from polypropylene glycols, like dipropylene glycol (e.g, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers,
monovalent to octadecavalent, preferably divalent to octadecavalent, more preferably divalent to hexavalent, even more preferably divalent, trivalent and tetravalent optionally OH substituted straight-chain, cyclic or branched, saturated, unsaturated or aromatic hydrocarbon groups, derived from ethers or esters of epoxy compounds, in total having more than three, preferred more than four carbon atoms, preferred glycidyl ethers, with alcohols, in particular methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxide)s, such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol, or derived from polypropylene glycols, like dipropylene glycol (e.g, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide)- and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid,
monovalent to octadecavalent, preferably divalent to octadecavalent, more preferably divalent to hexavalent, even more preferably divalent, trivalent and tetravalent optionally OH, amino or amido substituted straight-chain, cyclic or branched, saturated, unsaturated or aromatic hydrocarbon groups, formed from esters of halogenated carboxylic acids, preferred chloro carboxylic acids, in total having more than two, preferably more than three carbon atoms, such as chloroacetic acid, 3-chloropropionic acid, 4-chlorobutanoic acid or the respective bromo carboxylic acids, with ethers or esters of epoxy compounds, preferred glycidyl ethers, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2-hexanediol, 1,6-hexanediol, glycerol, diglycerol, triglycerol, and higher linear or branched oligoglycerols, trimethylol propane, castor oil (ricinoleic acid triglyceride), pentaerythritol, sorbitol, poly(alkylene oxide)s, such as (ethylene oxide)-, (propylene oxide)- and/or (butylene oxide)-based polyethers, in particular derived from polyethylene glycols, like diethylene glycol, triethylene glycol, tetraethylene glycol, and pentaethylene glycol, or derived from polypropylene glycols, like dipropylene glycol (in particular, derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide)- and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid,
monovalent to octadecavalent, preferably divalent to octadecavalent, more preferably divalent to hexavalent, even more preferably divalent, trivalent and tetravalent, optionally OH substituted straight-chain, cyclic or branched, saturated, unsaturated or aromatic hydrocarbon groups, formed from ethers of epoxy compounds, in total having more than seven, preferred more than eight carbon atoms, preferred glycidyl ethers, with di- to hexavalent carboxylic acids, in particular maleic acid, succinic acid, adipic acid, sebacic acid, itaconic acid, tartaric acid, trimellitic acid, fatty dimer acids, carboxyl (—C(O)OH) functionalized polyesters, in particular preferably formed by the condensation of di- to hexavalent carboxylic acids, e.g. maleic acid, succinic acid, adipic acid, sebacic acid, itaconic acid, tartaric acid, trimellitic acid, fatty dimer acids, with di- to hexavalent alcohols as outlined above or alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether and oligomeric glycerol glycidyl ethers, butanediol diglycidylether, in particular the condensation products of succinic acid, maleic acid and tartaric acid, fatty dimer acids with glycerol diglycidyl ether, polyesters, in particular preferably derived from oligomerized hydroxycarboxylic acids, in particular oligomerized lactic acid, 12-hydroxy stearic acid, lesquerolic acid, ricinoleic acid,
monovalent to octadecavalent, preferably divalent to octadecavalent, more preferably divalent to hexavalent, even more preferably divalent, trivalent and tetravalent, optionally OH substituted straight-chain, cyclic or branched, saturated, unsaturated or aromatic hydrocarbon groups,
   derived from esters of halogenated carboxylic acids, preferably chloro carboxylic acids, in total having more than five, preferred more than six carbon atoms such as esters of chloroacetic acid, 3-chloropropionic acid, 4-chlorobutanoic acid or the respective bromo carboxylic acids, with OH functionalized polyesters, in particular preferably formed by the condensation of di- to hexavalent carboxylic acids, e.g. maleic acid, succinic acid, adipic acid, sebacic acid, itaconic acid, tartaric acid, trimellitic acid, fatty dimer acids, with di- to hexavalent alcohols as outlined above or alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether and oligomeric glycerol glycidyl ethers, butanediol diglycidylether, in particular the condensation products of succinic acid, maleic acid and tartaric acid or fatty dimer acids with glycerol diglycidyl ether.

In a further preferred embodiment of the present invention, a compound of the formula:

R¹(—F)ₓ (I)

as defined above is provided, wherein
R¹ is selected from poly(alkylene oxide) groups, preferably poly(alkylene oxide) groups of the general formula (IX):

—[CH₂CH₂O]_{q1}—[CH₂CH(CH₃)O]ᵣ₁—[CH₂CH(C₂H₅)O]ₛ₁—{[CH₂CH₂]_{q2}—[CH₂CH(CH₃)]ᵣ₂—[CH₂CH(C₂H₅)]ₛ₂}— (IX)

with
q1=0 to 49, preferred 0 to 10, more preferred 1 to 10, even more preferred 1 to 5,
r1=0 to 32, preferred 0 to 10, more preferred 1 to 10, even more preferred 1 to 5,
   s1=0 to 24, preferred 0 to 10, more preferred 1 to 10, even more preferred 1 to 5,
   q2=0 or 1,
   r2=0 or 1,
   s2=0 or 1, and
   Σ(q2+r2+s2)=1,
   with the proviso that the sum of the carbon atoms in such poly(alkylene oxide) groups is 2 to 100, preferred 2 to 50, more preferred 2 to 30, even more preferred 2 to 20, specific 2 to 15, or
   R¹ is selected from divalent hydrocarbon groups derived from oligoglycerols of the general formula (X):

—[CH₂CH(R⁸)CH₂O]ₜ₁—[CH₂CH(R⁸)CH₂)]ₜ₂— (X)

with
t1=0 to 32, preferred 0 to 10, more preferred 1 to 10, even more preferred 1 to 5, specifically 1 and 2,
t2=1,
R⁸=OH or —O—C(O)—R⁶—(O—C(O)—R¹)ₘ—O—C(O)—R⁷, —O—C(O)—R⁶—N+(R³, R⁴, R⁵), wherein m, X, R³, R⁴, R⁵, R⁶ and R⁷ are as defined above,
with the proviso that the sum of the carbon atoms is 2 to 100, preferred 2 to 50, more preferred 2 to 30, even more preferred 2 to 20, specific 2 to 15, or R¹ is selected from divalent hydrocarbon groups, comprising at least one ester group of the general formula (XI):

—[CH₂CH₂O]_{q1}—R⁹—[CH₂CH₂O]_{q1}—[CH2CH2]_{q2}— (XI)

with q1 being the same or different and being as defined above and q2=1 and of the formula (XII)

—[CH₂CH(R⁸)CH₂O]ₜ₁—R⁹—[CH₂CH(R⁸)CH₂O]ₜ₁—[CH₂CH(R⁸)CH₂)]ₜ₂— (XII)

with t1, t2 and R⁸ as defined above, and
R⁹ being selected from —C(O)C(O)O—, —C(O)(CH₂)₁₋₈C(O)O—, such as being derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)—CH₂C(O)O—, —C(O)CH(OH)CH(OH)C(O)O—,
with the proviso that the sum of the carbon atoms in R⁹ is 2 to 100, preferred 2 to 50, more preferred 2 to 30, even more preferred 2 to 20, specifically 2 to 15.

According to this embodiment, preferably, q2=0, and one or two of q1, r1 and s1 are 0, and more preferably

q2=0, r1 and s1 are 0, or
q2=0, q1 and s1 are 0.

In a further preferred embodiment of the invention, the compound of the general formula (I) is as defined in the above embodiments, and R¹ contains one or more groups —O—, such as one to five. These groups —O— are preferably ether groups but can also form an ester group together with a carbonyl group, and preferably the group R¹ is substituted by one or more hydroxyl groups.

In a further preferred embodiment of the present invention, a compound of the formula

R¹(—F)ₓ (I)

as defined above is provided,
wherein
when one or more of the radicals R¹, R³, R⁴, R⁵ bonded to N⁺ contain the at least one moiety of the general formulas (III) or (IV)

(—X—C(O)—R⁶)ₘ—X—C(O)— (III) or

(—C(O)—X—R⁶)ₘ—C(O)—X— (IV),

preferably of the general formulas (IIIa) or (IVa)

(—X—C(O)—R⁶)ₘ—X—C(O)—R⁷ (IIIa), or

(—C(O)—X—R⁶)ₘ—C(O)—X—R⁷ (IVa)

with m=1-20 and X, R⁶ and R⁷ being as defined above, the at least one moiety has the structure of the general formulas (XIII) or (XIV)

—R¹⁰(—X—C(O)—R⁶)ₘ—X—C(O)— (XIII) or

—R¹⁰(—C(O)—X—R⁶)ₘ—C(O)—X— (XIV),

preferably of the general formulas (XIIIa) and (XIVa)

—R¹⁰(—X—C(O)—R⁶)ₘ—X—C(O)—R⁷ (XIIIa)

—R¹⁰(—C(O)—X—R⁶)ₘ—C(O)—X—R⁷ (XIVa).

wherein R¹⁰ is selected from divalent to octadecavalent, preferred divalent to decavalent, more preferred divalent to decavalent, specifically divalent, trivalent, tetravalent, pentavalent, hexavalent, heptavalent, octavalent, nonavalent, decavalent optionally substituted hydrocarbon radicals which have up to 200 carbon atoms, preferred 2 to 200 carbon atoms, more preferred 2 to 100 carbon atoms, even more preferred 2 to and 50 carbon atoms, specifically 2 to 20 carbon atoms, more specifically 2 to 10 carbon atoms and may contain optionally one or more groups selected from —O—, —NH—, —C(O)—, —C(S)—, tertiary amino groups

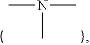

quaternary ammonium groups

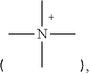

and can be substituted by —OH or halide groups, wherein the radical R¹⁰ cannot contain a combination of a —C(O)— group and a —O— group or a combination of a —C(O)— group and a —NH— or tertiary amino group forming an internal carboxylate group or an internal amide group,
and preferably R¹⁰ is represented by
divalent radicals, in particular-CH₂—, —CH₂CH₂—, —CH₂CH₂CH₂—, preferably derived from monochloro carboxylic acids such as chloro acetic acid, chloro propionic acid, chlorobutanoic acid, or preferably derived from tertiary amino alcohols such as N,N-dimethylethanolamine, N,N-dimethylpropanolamine,
trivalent radicals, preferably derived from partial esters of said monochloro carboxylic acids, in particular esters of chloro acetic acid, with trivalent alcohols, in particular glycerol, trimethylol propane, castor oil (ricinoleic acid triglyceride) or preferably derived from tertiary amino alcohols such as N,N,N′-trimethylaminoethyl-ethanolamine, or preferably derived from esters of tertiary amino alcohols, in particular N,N-dimethylethanolamine, N,N-dimethylpropanolamine, with dihydroxy carboxylic acids, in particular 2,2-hydroxymethyl propanoic acid,
tetravalent to hexavalent radicals, preferably derived from partial esters of said monochloro carboxylic acids, in particular esters of chloro acetic acid, with tetravalent alcohols, in particular erythritol, pentaerythritol, diglycerol, pentavalent alcohols, in particular xylitol, triglycerol, hexavalent alcohols, in particular sorbitol, tetraglycerol, or preferably derived from esters of tertiary amino alcohols, in particular N,N-dimethylethanolamine, N,N-dimethylpropanolamine, with dendrimeric oligomers of dihydroxy carboxylic acid oligomers, in particular dendrimeric oligomers of 2,2-hydroxymethyl propanoic acid, heptavalent to octadecavalent radicals, preferably derived from partial esters of said monochloro carboxylic acids, in particular esters of chloro acetic acid, with heptavalent to octadecavalent alcohols, in particular pentaglycerol to hexadecaglycerol,
   with the proviso that R¹⁰ is linked by a single bond to a N⁺ moiety and is linked to at least one radical, preferred one, two, three, four radicals of the structures of the general formulas (III) or (IV)

(—X—C(O)—R⁶)ₘ—X—C(O)— (III) or

(—C(O)—X—R⁶)ₘ—C(O)—X— (IV),

and more preferred to one, two, three, four radicals of the general formulas (IIIa) or (IVa)

(—X—C(O)—R⁶)ₘ—X—C(O)—R⁷ (IIIa)

(—C(O)—X—R⁶)ₘ—C(O)—X—R⁷ (IVa)

with X, m, R¹¹, R⁶, R⁷ as defined above.

According to the above embodiment, it is preferred when X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom,
and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl,
and m is 1,2, 3, 4 or 5.

It is most preferred according to this embodiment when X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid, and
m is 1, 2, 3, 4 or 5.

In a further preferred embodiment of the present invention, a compound of the formula

R¹(—F)ₓ (I)

as defined in the previous embodiment
is provided,
wherein for the moiety

—R¹(—X—C(O)—R⁶)ₘ—X—C(O)— (XIII) or preferably

R¹(—X—C(O)—R⁶)ₘ—X—C(O)R⁷ (XIIIa)

with the X adjacent to R¹⁰ being 0,
R¹⁰ is derived from
mono or di-(chloroacetic acid) esters of glycerol or castor oil (ricinoleic acid triglyceride), and is bonded to one or two moieties (—X—C(O)—R⁶)ₘ—X—C(O)—, preferably (—X—C(O)—R⁶)ₘ—O—C(O)—R⁷, in total,
or R¹⁰ is derived from
   esters of tertiary aminoalcohols, in particular N,N-dimethylethanolamine, N,N-dimethylpropanolamine, N,N,N′-trimethylaminoethyl-ethanolamine, and is bonded to one moiety (—X—C(O)—R⁶)ₘ—O—C(O)—R⁷, preferably (—X—C(O)—R⁶)ₘ—O—C(O)—R⁷, in total,
   or R¹⁰ is derived from esters of tertiary aminoalcohols, in particular N,N-dimethylethanolamine, N,N-dimethylpropanolamine, with dihydroxy carboxylic acids, in particular 2,2-hydroxymethyl propanoic acid, and is bonded to two moieties (—X—C(O)—R⁶)ₘ—X—C(O)—, preferably (—X—C(O)—R⁶)ₘ—O—C(O)—R⁷ in total,
   or R¹⁰ is derived from esters of tertiary aminoalcohols, in particular N,N-dimethylethanolamine, N,N-dimethylpropanolamine, with dendrimeric oligomers of dihydroxy carboxylic acids, in particular dendrimeric oligomers of 2,2-hydroxymethyl propanoic acid, and is bonded to more than two, preferred three or four moieties (—X—C(O)—R⁶)ₘ—X—C(O)—, preferably (—X—C(O)—R⁶)ₘ—O—C(O)—R⁷ in total, and for the moiety

—R¹(—X—C(O)—R⁶)ₘ—X—C(O)— (XIII) or preferably

—R¹(—X—C(O)—R⁶)ₘ—X—C(O)—R⁷ (XIIIa)

with the X adjacent to R¹⁰ being N
R¹⁰ is derived from
tertiary-primary amines, in particular N,N-dimethyl-1,3-propanediamine, N-methyl-N′-aminopropyl-piperazine, tertiary-secondary amines, in particular N-methylpiperazine, and
   for both types of moieties
   R⁶ is as defined above and preferably derived from lactic acid, ricinoleic acid, lesquerolic acid 10-hydroxy stearic acid, 12-hydroxy stearic acid, 14-hydroxy tetradecanoic acid, most preferably derived from ricinoleic acid or lesquerolic acid,
   R⁷ is as defined above and preferably derived from octadecanoic acid, eicosanoic acid, docosanoic acid, 2-ethyl hexanoic acid, 2,2-dimethyl propionic acid, neodecanoic acid, oleic acid, m=1 to 20, preferred 1 to 10, more preferred 1 to 6, even more preferred 2 to 6, specifically 1, 2, 3, 4, 5, 6, 7, and the total number of carbon atoms in R⁶+R⁷ (Σ carbon atoms of R⁶ and R⁷) is 19 to 300, preferred 25 to 300, more preferred 35 to 300, even more preferred 50 to 300, specifically 35 to 200, more specifically 35 to 150, even more specifically 50 to 150,
   R¹¹ is preferably selected from the group consisting of hydrogen or a ring-forming alkylene, in particular derived from a piperazine ring.

In a further preferred embodiment of the present invention, a compound of the formula

R¹(—F)ₓ (I)

as defined in the two previous embodiments
is provided,
wherein
R⁶ is as defined above and preferably derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid,
R⁷ is as defined above and preferably derived from octadecanoic acid, eicosanoic acid, docosanoic acid, 2-ethyl hexanoic acid, 2,2-dimethyl propionic acid, neodecanoic acid, oleic acid, and for the moieties

—R¹⁰(—X—C(O)—R⁶)ₘ—X—C(O)— (XIII) and

—R¹⁰(—C(O)—X—R⁶)ₘ—C(O)—X— (XIV),

preferably for the moieties

—R¹⁰(—X—C(O)—R⁶)ₘ—X—C(O)—R⁷ (XIIIa) and

—R¹⁰(—C(O)—X—R⁶)ₘ—C(O)—X—R⁷ (XIVa)

the sequence of the radicals R⁶ and if present R⁷ within the ester segments is either random or block-like, and for block-like sequences, the compound contains the structures of the general formulas (XV) or (XVI)

—R¹⁰—X—C(O)—R⁶(—X—C(O)—R⁶¹)ₘ₁(—X—C(O)—R⁶²)ₘ₂—X—C(O)— (XV)

—R¹⁰—C(O)—X—R⁶(—C(O)—X—R⁶¹)ₘ₁(—C(O)—X—R⁶²)ₘ₂—C(O)—X— (XVI),

preferably of the general formulas (XVa) or (XVIa)

—R¹⁰—X—C(O)—R⁶(—X—C(O)—R⁶¹)ₘ₁(—X—C(O)—R⁶²)ₘ₂—X—C(O)—R⁷ (XVa)

—R¹⁰—C(O)—X—R⁶(—C(O)—X—R⁶¹)ₘ₁(—C(O)—X—R⁶²)ₘ₂—C(O)—X—R⁷ (XVIa)

with
R⁶¹ and R⁶² being selected from R⁶,
m1=0 to 20, preferred 0 to 10, more preferred 0 to 6, even more preferred 1 to 6, specifically 0, 1, 2, 3, 4, 5, 6,
m2=0 to 20, preferred 0 to 10, more preferred 0 to 6, even more preferred 1 to 6, specifically 0, 1, 2, 3, 4, 5, 6,
m=(m1+m2)+1,
m=1 to 20, preferred 1 to 10, more preferred 1 to 6, even more preferred 1 to 6, specifically 1, 2, 3, 4, 5, 6, 7 and the total number of carbon atoms in R⁶+R⁷ (Σ carbon atoms of R⁶ and R⁷) being 19 to 300, preferably 25 to 300, more preferably 35 to 300, even more preferably 50 to 300, specifically 35 to 200, more specifically 35 to 150, even more specifically 50 to 150, wherein the sequences of the structures of the general formulas (XV) and (XVI) are preferably selected from

| | | R⁶² at the terminus |
|---|---|---|
| R⁶ adjacent to R¹⁰ | R⁶¹ adjacent to R⁶ | opposite of R¹⁰ |
| derived from | derived from | derived from |
| unsaturated acid, in | unsaturated acid, in | unsaturated acid, in |
| particular ricinoleic | particular ricinoleic | particular ricinoleic |
| acid or lesquerolic | acid or lesquerolic | acid or lesquerolic |
| acid | acid | acid |
| unsaturated acid, in | unsaturated acid, in | saturated acid, in |
| particular ricinoleic | particular ricinoleic | particular 12- |
| acid or lesquerolic | acid or lesquerolic | hydroxystearic acid |
| acid | acid | |
| unsaturated acid, in | saturated acid, in | saturated acid, in |
| particular ricinoleic | particular 12- | particular 12- |
| acid or lesquerolic | hydroxystearic acid | hydroxystearic acid |
| acid | | |
| saturated acid, in | saturated acid, in | saturated acid, in |
| particular 12- | particular 12- | particular 12- |
| hydroxystearic acid | hydroxystearic acid | hydroxystearic acid |
| saturated acid, in | saturated acid, in | unsaturated acid, in |
| particular 12- | particular 12- | particular ricinoleic |
| hydroxystearic acid | hydroxystearic acid | acid or lesquerolic |
| | | acid |
| saturated acid, i.e. | unsaturated acid, in | unsaturated acid, in |
| 12-hydroxystearic | particular ricinoleic | particular ricinoleic |
| acid | acid or lesquerolic | acid or lesquerolic |
| | acid | acid |
| saturated acid, in | unsaturated acid, in | saturated acid, in |
| particular 12- | particular ricinoleic | particular 12- |
| hydroxystearic acid | acid or lesquerolic | hydroxystearic aicd |
| | acid | |
| unsaturated acid, in | saturated acid, in | unsaturated acid, in |
| particular ricinoleic | particular 12- | particular ricinoleic |
| acid or lesquerolic | hydroxystearic acid | acid or lesquerolic |
| acid | | acid |

and wherein the sequences of the structures of the general formulas (XVa) and (XVIa) are preferably selected from

| R⁶ adjacent to R¹⁰ | R⁶¹ adjacent to R⁶ | R⁶² adjacent to R⁷ | |
|---|---|---|---|
| derived from | derived from | derived from | R⁷ derived from |
| unsaturated acid, in | unsaturated acid, in | unsaturated acid, in | unsaturated acid, in |
| particular ricinoleic | particular ricinoleic | particular ricinoleic | particular oleic aicd |
| acid or lesquerolic | acid or lesquerolic | acid or lesquerolic | |
| acid | acid | acid | |
| unsaturated acid, in | unsaturated acid, in | unsaturated acid, in | saturated acid, in |
| particular ricinoleic | particular ricinoleic | particular ricinoleic | particular |
| acid or lesquerolic | acid or lesquerolic | acid or lesquerolic | octadecanoic acid, |
| acid | acid | acid | neodecanoic acid |
| unsaturated acid, in | unsaturated acid, in | saturated acid, in | saturated acid, in |
| particular ricinoleic | particular ricinoleic | particular 12- | particular |
| acid or lesquerolic | acid or lesquerolic | hydroxystearic acid | octadecanoic acid, |
| acid | acid | | neodecanoic acid |
| unsaturated acid, in | saturated acid, in | saturated acid, in | saturated acid, |
| particular ricinoleic | particular 12- | particular 12- | i.e. octadecanoic |
| acid or lesquerolic | hydroxystearic acid | hydroxystearic acid | acid, neodecanoic |
| acid | | | acid |
| saturated acid, in | saturated acid, in | saturated acid, in | saturated acid, in |
| particular 12- | particular 12- | particular 12- | particular |
| hydroxystearic acid | hydroxystearic acid | hydroxystearic acid | octadecanoic acid, |
| | | | neodecanoic acid |
| saturated acid, in | saturated acid, in | saturated acid, in | unsaturated acid, in |
| particular 12- | particular 12- | particular 12- | particular oleic acid |
| hydroxystearic acid | hydroxystearic acid | hydroxystearic acid | |
| saturated acid, in | saturated acid, in | unsaturated acid, in | unsaturated acid, in |
| particular 12- | particular 12- | particular ricinoleic | particular oleic acid |
| hydroxystearic acid | hydroxystearic acid | acid or lesquerolic | |
| | | acid | |
| saturated acid, i.e. | unsaturated acid, in | unsaturated acid, in | unsaturated acid, in |
| 12-hydroxystearic | particular ricinoleic | particular ricinoleic | particular oleic acid |
| acid | acid or lesquerolic | acid or lesquerolic | |
| | acid | acid | |
| saturated acid, in | unsaturated acid, in | saturated acid, in | unsaturated acid, in |
| particular 12- | particular ricinoleic | particular 12- | particular oleic acid |
| hydroxystearic acid | acid or lesquerolic | hydroxystearic aicd | |
| | acid | | |
| unsaturated acid, in | saturated acid, in | unsaturated acid, in | saturated acid, in |
| particular ricinoleic | particular 12- | particular ricinoleic | particular |
| acid or lesquerolic | hydroxystearic acid | acid or lesquerolic | octadecanoic acid, |
| acid | | acid | neodecanoic acid. |

According to this embodiment, it is particularly preferred when the combination of R⁶, R⁶¹, R⁶² or of R⁶, R⁶¹, R⁶² and R⁷ is selected according to the specific compounds named in one of the lines of the table above

It is in general within the scope of the invention to incorporate into the moieties

(—X—C(O)—R⁶)ₘ—X—C(O)— (III) and

(—C(O)—X—R⁶)ₘ—C(O)—X— (IV)

R⁶-containing ester segments which are either monomodal or polymodal with respect to their molecular weight distribution. Within the context of the present invention the term monomodal means that 80% of the ester segments have the same molecular weight. The term polymodal means that none of the individual ester segments reaches 80% of the total composition.

It is also generally within the scope of the invention to incorporate into the moieties

(—X—C(O)—R⁶)ₘ—X—C(O)—R⁷ (IIIa) and

(—C(O)—X—R⁶)ₘ—C(O)—X—R⁷ (IVa)

R⁶ and R⁷ containing ester segments which are either monomodal or polymodal with respect to their molecular weight distribution.

It is in particular within the scope of the present invention to incorporate into the moieties

—R¹⁰(—X—C(O)—R⁶)ₘ—X—C(O)— (XIII) and

—R¹⁰(—C(O)—X—R⁶)ₘ—C(O)—X— (XIV),

in particular into the moieties

—R¹⁰(—X—C(O)—R⁶)ₘ—X—C(O)—R⁷ (XIIIa) and

—R¹⁰(—C(O)—X—R⁶)ₘ—C(O)—X—R⁷ (XIVa)

R⁶ and if present R⁷-containing ester segments which are either monomodal or polymodal with respect to their molecular weight distribution. The terms “monomodal” and “polymodal” have the meaning as defined above.

Therein, according to this embodiment, it is preferred when X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl, and m is 1,2, 3, 4 or 5.

It is most preferred according to this embodiment when X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid, and m is 1, 2, 3, 4 or 5.

It is also within the scope of the invention to incorporate into the compounds according to the invention, which can be mono-, di- and polyquaternary compounds, one type or more than one type (mixtures of different structures) of the moieties

(—X—C(O)—R⁶)ₘ—X—C(O)— (III) and

(—C(O)—X—R⁶)ₘ—C(O)—X— (IV).

Accordingly, it is also within the scope of the invention to incorporate into the compounds according to the invention one type or more than one type (mixture of different structures) of the moieties

(—X—C(O)—R⁶)ₘ—X—C(O)—R⁷ (IIIa) and

(—C(O)—X—R⁶)ₘ—C(O)—X—R⁷ (IVa),

and it is also within the scope of the invention to incorporate into the compounds according to the invention one type or more than one type (mixture of different structures) of the moieties

—R¹⁰(—X—C(O)—R⁶)ₘ—X—C(O)— (XIII) and

—R¹⁰(—C(O)—X—R⁶)ₘ—C(O)—X— (XIV),

and in particular it is within the scope of the invention to incorporate into the compounds according to the invention one type or more than one type (mixture of different structures) of the moieties

—R¹⁰(—X—C(O)—R⁶)ₘ—X—C(O)—R⁷ (XIIIa) and

—R¹⁰(—C(O)—X—R⁶)ₘ—C(O)—X—R⁷ (XIVa).

According to the invention the R⁶-containing ester elements in the moieties

(—X—C(O)—R⁶)ₘ—X—C(O)— (III),

(—C(O)—X—R⁶)ₘ—C(O)—X— (IV),

and in particular the R⁶- and R⁷-containing ester elements in the moieties

(—X—C(O)—R⁶)ₘ—X—C(O)—R⁷ (IIIa) and

(—C(O)—X—R⁶)ₘ—C(O)—X—R⁷ (IVa),

and even more in particular in the moieties

—R¹⁰(—X—C(O)—R⁶)ₘ—X—C(O)— (XIII),

—R¹⁰(—C(O)—X—R⁶)ₘ—C(O)—X— (XIV)

—R¹⁰(—X—C(O)—R⁶)ₘ—X—C(O)—R⁷ (XIIIa) and

—R¹⁰(—C(O)—X—R⁶)ₘ—C(O)—X—R⁷ (XIVa),

for instance in the moieties

—R¹⁰—O—C(O)—R⁶—(O—C(O)—R⁶)ₘ—O—C(O)—,

—R¹⁰—NR¹—C(O)—R⁶—(O—C(O)—R⁶)ₘ—O—C(O)—,

—R¹⁰—O—C(O)—R⁶—(O—C(O)—R⁶)ₘ—O—C(O)—R⁷ and

—R¹⁰—NR¹—C(O)—R⁶—(O—C(O)—R⁶)ₘ—O—C(O)—R⁷,

can be synthesized from the corresponding carboxylic acids by esterification using methods known in the prior art. In a preferred embodiment these esterifications can be carried out thermally at 150-350° C. preferred at 180 to 250° C. under reduced pressure (US2011/0282084, GB 841554, DE 694943). Additionally, catalysts can be used to run the esterifications (EP 3009494, WO 2012069386, DD 150064, CH 151317, T. A. Isbell, Grasas y Aceites, 2011, 62(1), 8-20). In another preferred embodiment, enzymes are used to condensate the carboxylic acids (JP 05304966, JP 05211878, JP 01016591, A. Bodalo et al., Biochem. Eng. J., 2008, 39(3), 450-456, A. Bodalo et al., Biochem. Eng. J. 2005, 26(2-3), 155-158, Y. Yasuko et al., J. Am. Oil Chem. Soc., 1997, 74(3), 261-267). In general, the above described methods provide polymodal condensates.

In general monomodal condensates can be synthesized by a condensation sequence based on the stepwise esterification of carboxylic acid anhydrides (K. Meier, Farbe und Lack, 1951, 57, 437-439, F. H. H. Valentin, J. South African Chem. Inst. 1949, 2, 59-61) or, preferred, carboxylic acid chlorides (K. D. Pathak et al., J. Scientific & Industrial Research, 1955, 14B, 637-639) with OH groups of hydroxylated carboxylic acids and their derivatives.

Repetitons of a cycle based on an esterification and an acid chloride synthesis provide in general monomodal ester condensates. Further details will be outlined in the example section.

Below, a schematic representation of a sequence for the synthesis of ester condensates based on the stepwise esterification of carboxylic acid chlorides and OH groups of hydroxylated carboxylic acids and their derivatives is given:

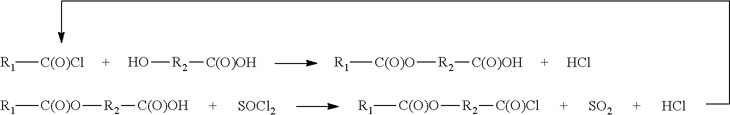

Herein, the arrow indicates that the product obtained by esterification of an acyl chloride of a fatty acid R₁—C(O)CL by reaction with the hydroxyl-carboxylic acid HO—R₂—C(O)OH and subsequent formation of an acyl chloride by reaction with SOCl₂ can be resubmitted to such reaction sequence. Accordingly, in the next reaction sequence R₁ of the starting material R₁—C(O)CL is “R₁—C(O)O—R₂” of the previous reaction sequence. Thus, estolide structures can be obtained in an iterative manner, and the number of fatty acid residues comprised by the final estolide moiety is determined by the number of iteration steps of the circular process.

Carboxylic acids free of OH groups terminate the chains of the ester condensates. Monohydroxy carboxylic acids extend the chains in the ester condensates. Generally, di- and polyhydroxy carboxylic acids provide branched and dendrimeric (self repeating) elements within the ester condensates.

In a further preferred embodiment of the invention, a compound of the formula

R¹(—F)ₓ (I)

as defined above is provided, wherein
low melting and high melting fatty acids≥C5 are specifically positioned within the R⁶ containing ester elements of the general formulas (III) and (IV)

(—X—C(O)—R⁶)ₘ—X—C(O)— (III)

(—C(O)—X—R⁶)ₘ—C(O)—X— (IV), in particular

within the R⁶- and R⁷-containing ester elements of the general formulas (IIIa) and (IVa)

(—X—C(O)—R⁶)ₘ—X—C(O)—R⁷ (IIIa) and

(—C(O)—X—R⁶)ₘ—C(O)—X—R⁷ (IVa) or

within the R⁶ containing ester elements of the general formulas (XIII) and (XIV)

—R¹⁰(—X—C(O)—R⁶)ₘ—X—C(O)— (XIII)

—R¹⁰(—C(O)—X—R⁶)ₘ—C(O)—X—(XIV), in particular

within the R⁶- and R⁷-containing ester elements of the general formulas (XIIIa) and (XIVa)

—R¹⁰(—X—C(O)—R⁶)ₘ—X—C(O)—R⁷ (XIIIa)

—R¹⁰(—C(O)—X—R⁶)ₘ—C(O)—X—R⁷ (XIVa).

It is in general within the scope of the invention that low melting and high melting fatty acids C5 are specifically positioned independently for individual ester groups of moieties selected from the moieties of the general formulas (III), (IV), (IIIa), (IVa), (XIII), (XIV), (XIIIa) and (XIVa) present in the compounds of the general formula (I). For instance, it is according to this embodiment of the invention if a number of moieties of the general formula (III) displays the specific positioning of low melting fatty acids and high melting fatty acid scaffolds as described in the following while other moieties of the general formula (III) do not. This may be in particular the case for moieties present in different residues R¹, R², R³, R⁴ and R⁵ as defined above.

Within the frame of the present invention low melting fatty acids≥C5 are defined by a melting point≤40° C. Preferred examples are in particular oleic acid, lesquerolic acid, ricinoleic acid, octanoic acid, decanoic acid, pivalinic acid, neodecanoic acid.

Within the frame of the present invention high melting fatty acids≥C5 are defined by a melting point>40° C. Preferred examples are in particular dodecanoic acid, tetradecanoic acid, hexadecanoic acid, octadecanoic acid, arachidic acid, behenic acid, 10-hydroxy octadecanoic acid, 12-hydroxy octadecanoic acid acid, 14-hydroxy tetradecanoic acid.

The corresponding melting points can be taken from the literature (G. Knothe et al., J Am Oil Chem Soc, 2009, 86, 844-856).

In a further preferred embodiment according to the invention, a compound of the formula

R¹(—F)ₓ (I)

as defined above is provided, wherein
at least one, preferred more than one, more preferred one, two or three low melting fatty acids≥C5 each forming a group R⁶ are positioned at the one terminus of a R⁶-containing ester element of the formula (III) or (IV), while at least one, preferred more than one, more preferred one, two or three high melting fatty acids≥C5 form the radical or radicals R⁶ at the opposite terminus of the ester element of the formula (III) or (IV), or in such a manner that at least one, preferred more than one, more preferred one, two or three high melting fatty acids≥C5 each forming a group R⁶ are positioned at the one terminus of a R⁶-containing ester element of the formula (III) or (IV), while at least one, preferred more than one, more preferred one, two or three low melting fatty acids≥C5 form the radical or radicals R⁶ at the opposite terminus of the ester element of the formula (III) or (IV), or
at least one, preferred more than one, more preferred one, two or three low melting fatty acids≥C5 each forming a group R⁶ are contained in the radical or the radicals R⁶ adjacent to R⁷, while at least one, preferred more than one, more preferred one, two or three high melting fatty acids≥C5 form the radical or radicals R⁶ at the opposite terminus of a R⁶- and R⁷-containing ester element of the formula (IIIa) or (IVa), or in such a manner that least one, preferred more than one, more preferred one, two or three high melting fatty acids≥C5 each forming R⁶ form the radical or radicals R⁶ adjacent to R⁷, while at least one, preferred more than one, more preferred one, two or three low melting fatty acids≥C5 form the radical or radicals R⁶ at the opposite terminus of a R⁶- and R⁷-containing ester element of the formula (IIIa) or (IVa), or
at least one, preferred more than one, more preferred one, two or three low melting fatty acids≥C5 each forming a group R⁶ are positioned adjacent to the radical R¹⁰ while at least one, preferred more than one, more preferred one, two or three high melting fatty acids≥C5 form the radical or the radicals R⁶ at the opposite terminus of the ester element of the formula (XIII) or (XIV), or in such a manner that least one, preferred more than one, more preferred one, two or three high melting fatty acids≥C5 each forming R⁶ form the radical or radicals R⁶ adjacent to the radical R¹⁰, while at least one, preferred more than one, more preferred one, two or three low melting fatty acids≥C5 form the radical or radicals R⁶ at the opposite terminus of a R⁶- and R⁷-containing ester element of the formula (XIII) or (XIV), or
at least one, preferred more than one, more preferred one, two or three low melting fatty acids≥C5 each forming a group R⁶ are positioned adjacent to the radical R¹⁰ while at least one, preferred more than one, more preferred one, two or three high melting fatty acids≥C5 form the radical or the radicals R⁶ adjacent to R⁷ in the moieties of the formulas (XIIIa) or (XIVa), or in such a manner that at least one, preferred more than one, more preferred one, two or three high melting fatty acids≥C5 each forming R⁶ form the radical or radicals R⁶ adjacent to the radical R¹⁰ while at least one, preferred more than one, more preferred one, two or three low melting fatty acids≥C5 form the radical or radicals R⁶ adjacent to R⁷ in the moieties of the formulas (XIIIa) or (XIVa).

As already stated above, the specific positioning of high and low melting fatty acids may be independently varied for each individual R⁶-containing ester moiety of the formulas given above.

The above outlined preferred embodiments allow the incorporation of R⁶- as well as R⁶- and R⁷-containing ester elements having a locally varying tendency towards crystallization, viscosity build up and phase formation over the whole length of these ester elements in the moieties of the general formulas

(—X—C(O)—R⁶)ₘ—X—C(O)— (III)

(—C(O)—X—R⁶)ₘ—C(O)—X— (IV),

(—X—C(O)—R⁶)ₘ—X—C(O)—R⁷ (IIIa) and

(—C(O)—X—R⁶)ₘ—C(O)—X—R⁷ (IVa),

—R¹⁰(—X—C(O)—R⁶)ₘ—X—C(O)— (XIII),

—R¹⁰(—C(O)—X—R⁶)ₘ—C(O)—X— (XIV),

—R¹(—X—C(O)—R⁶)ₘ—X—C(O)—R⁷ (XIIIa) and

—R¹⁰(—C(O)—X—R⁶)ₘ—C(O)—X—R⁷ (XIVa),

in particular in

—R¹⁰—C(O)—R⁶—(O—C(O)—R⁶)ₘ—O—C(O)—,

—R¹⁰—NR¹—C(O)—R⁶—(O—C(O)—R⁶)ₘ—O—C(O)—,

—R¹⁰—C(O)—R⁶—(O—C(O)—R⁶)ₘ—O—C(O)—R⁷ and

—R¹⁰—NR¹—C(O)—R⁶—(O—C(O)—R⁶)ₘ—O—C(O)—R⁷.

On purpose combination of the above-mentioned carboxylic acids and synthetic concepts gives access to ester condensates having defined molecular weights, molecular weight distributions, carboxylic acid sequences and properties such as viscosity.

In general, the radical R¹⁰ can be linked to the R⁶ and R⁶- and R⁷-containing ester elements in

—R¹(—X—C(O)—R⁶)ₘ—X—C(O)—R⁷ (XIII),

—R¹⁰(—C(O)—X—R⁶)ₘ—C(O)—X—R⁷ (XIV),

—R¹⁰(—X—C(O)—R⁶)ₘ—X—C(O)—R⁷ (XIIIa) and

—R¹(—C(O)—X—R⁶)ₘ—C(O)—X—R⁷ (XIVa),

in particular in

—R¹⁰—C(O)—R⁶—(O—C(O)—R⁶)ₘ—O—C(O)— and

—R¹⁰—NR¹—C(O)—R⁶—(O—C(O)—R⁶)ₘ—O—C(O)—,

—R¹⁰—C(O)—R⁶—(O—C(O)—R⁶)ₘ—O—C(O)—R⁷ and

—R¹⁰—NR¹—C(O)—R⁶—(O—C(O)—R⁶)ₘ—O—C(O)—R⁷

in different ways.

In a preferred embodiment according to the invention, a compound of the general formula (I) as defined in the previous embodiments is provided, wherein R¹⁰, preferably in

—R¹⁰(—X—C(O)—R⁶)ₘ—X—C(O)— (XIII)

—R¹⁰(—X—C(O)—R⁶)ₘ—X—C(O)—R⁷ (XIIIa),

more preferably in

—R¹⁰—C(O)—R⁶—(O—C(O)—R⁶)ₘ—O—C(O)—,

—R¹⁰—C(O)—R⁶—(O—C(O)—R⁶)ₘ—O—C(O)—R⁷,

is derived from mono or di-(chloroacetic acid) esters of glycerol or castor oil (ricinoleic acid triglyceride) containing two or more moieties —O—C(O)—R⁶—(O—C(O)—R⁶)ₘ—O—C(O)— or is bonded to one or two moieties —O—C(O)—R⁶—(O—C(O)—R⁶)ₘ—O—C(O)—R⁷ in total.

According to this embodiment, it is preferred when X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl, and m is 1,2, 3, 4 or 5.

It is most preferred according to this embodiment when X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid, and m is 1,2, 3, 4 or 5.

The esterification of hydroxylated fatty acids or hydroxylated glycerol fatty acid derivatives with chloro acetic acid (R. Oda, Kogyo Kagaku Zasshi, 1933, 36, suppl. Binding 496-497) or chloro acetic acid chloride (EP 0283994, A. Baydar et al., Int. J. Cosmet. Sci., 1991, 13(4), 169-190), is described in the prior art. Further details are outlined in the example section.

In another preferred embodiment according to the invention, a compound of the general formula (I) as defined above is provided, wherein R¹⁰, preferably in

—R¹⁰(—X—C(O)—R⁶)ₘ,—X—C(O)— (XIII) and

—R¹⁰(—X—C(O)—R⁶)ₘ—X—C(O)—R⁷ (XIIIa),

more preferably in

—R¹⁰—C(O)—R⁶—(O—C(O)—R⁶)ₘ—O—C(O)— and

—R¹⁰—C(O)—R⁶—(O—C(O)—R⁶)ₘ—O—C(O)—R⁷,

is derived from esters of tertiary aminoalcohols, in particular N,N-dimethylethanolamine, N,N-dimethylpropanolamine, N,N,N′-trimethylaminoethyl-ethanolamine with esters of hydroxylated carboxylic acids.

The esterification of tertiary amino groups containing alcohols with carboxylic acid chlorides is described in the prior art (U.S. Pat. No. 2,460,182). Further details are described in the example section.

In another preferred embodiment R¹⁰, preferably in

—R¹⁰(—X—C(O)—R⁶)ₘ,—X—C(O)— (XIII) and

—R¹⁰(—X—C(O)—R⁶)ₘ—X—C(O)—R⁷ (XIIIa),

more preferably in

—R¹⁰—NR¹—C(O)—R⁶—(O—C(O)—R⁶)ₘ—O—C(O)— or

—R¹⁰—NR¹—C(O)—R⁶—(O—C(O)—R⁶)ₘ—O—C(O)—R⁷,

is derived from amides of tertiary-primary amines, in particular N,N-dimethyl-1,3-propanediamine, N-methyl-N′-aminopropyl-piperazine, tertiary-secondary amines, in particular N-methylpiperazine.

The synthesis of tertiary amino groups containing fatty amides starting from fatty acid esters (U.S. Pat. No. 4,221,733) or the free fatty acids (U.S. Pat. No. 3,768,646) is described in the prior art. Further details are described in the example section.

In a preferred embodiment of the invention, a compound of the general formula (I) is provided, wherein the moiety R¹ of the compound of the general formula (I) as defined above is formed by the reaction of halogenated carboxylic acids, preferred chloro acetic acid, with OH functionalized hydrocarbons. The synthesis of chloro acetic acid esters starting from chloro acetic acid or chloro acetic acid chloride and OH functionalized hydrocarbons is described in the prior art (R. Oda, Kogyo Kagaku Zasshi, 1933, 36, suppl. Binding 496-497, WO 0210257).

In another preferred embodiment according to the invention R¹ of the compound of the general formula (I) as defined above is formed by the reaction of epoxy derivatives, preferred glycidyl ether or glycidyl ester derivatives, of hydrocarbons with difunctional carboxylic acids. These glycidyl ether or glycidyl ester derivatives are either commercial or can be synthesized from the corresponding alcohol or carboxylic acid precursors. Preferred commercial epoxy derivatives are the Denacol types (Nagase) or Heloxy modifiers (Hexion), i.e. the corresponding derivatives based on 1,4-butanediol, glycerol, oligoglycerol, castor oil and dimer acid. The synthesis of glycidyl ethers or glycidyl esters is described in the prior art (GB 763559, U.S. Pat. Nos. 3,766,221, 5,420,312, WO 2012041816).

In another preferred embodiment of the invention, a compound of the general formula (I) is provided, wherein R¹ of the compound of the general formula (I) as defined above is formed by the reaction of esters of halogenated carboxylic acids, preferred chloro acetic acid, with epoxy functionalized hydrocarbons or epoxy esters based on epoxy functionalized hydrocarbons with difunctional carboxylic acids. The synthesis of this type of esters is described in US 2018/0016397.

In general, the counter ions A⁻ of the ammonium anions of the compound of the general formula (I) as defined above are selected from mono- to trivalent inorganic anions and mono- to 30000-valent, preferably mono- to kiliavalent organic anions, which are preferably selected from the group consisting of halides, such as chloride, bromide, iodide, sulphate, phosphate, phosphonate, sulphonate, methosulphate, carboxylates, such as acetate, propionate, lactate, octanoate, 2-ethyl-hexanoate, dodecanoate, hexadecanoate, octadecanoate, oleate, ricinoleate, 12-hydroxy-octadecanoate, succinate, maleate, tartrate, polyethercarboxylate, polymeric fatty acid carboxylates of the type

R¹[(—C(O)—X—R⁶)ₘ—C(O)—X—R⁷]ₓ or

R¹[(X—C(O)—R⁶)ₘ—X—C(O)—R⁷]ₓ,

wherein either R¹ or at least one of R⁷, or both R¹ and at least one of R⁷ bear one or more carboxylate groups, preferably with X=O,
in particular
linear polymeric fatty acid carboxylates of the type

—O—C(O)—R⁶(—X—C(O)—R⁶)ₘ₋₁—X—C(O)—R⁷, preferably

—O—C(O)—R⁶—(O—C(O)—R⁶)ₘ—O—C(O)—R⁷,

branched linear polymeric fatty acid carboxylates,
i.e. derived from branched poly fatty acid structures, in particular branched linear polymeric fatty acid carboxylates derived from partial esters of polyfunctional carboxylic acids, in particular of the dicarboxylic acids succinic acid and maleic acid, with castor oil or lesquerella oil, such as

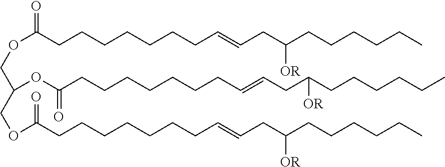

with one

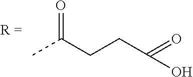

and the remaining two

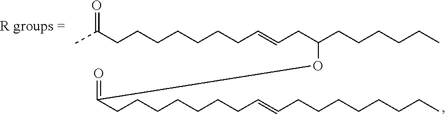

dendritic polymeric fatty acid carboxylates,
i.e. derived from dendritic poly fatty acid structures,
or of the types

X—R⁶(—C(O)—X—R⁶)ₘ₋₁C(O)—X—R⁷ or

R⁶(—C(O)—X—R⁶)ₘ₋₁C(O)—X—R⁷,

wherein in the two latter types the R⁷ group bears at least one anionic carboxylate group, or of the type

R¹[(—C(O)—X—Re)ₘ—C(O)O⁻]ₓ, such as

wherein X, R¹, R⁶, R⁷, m and x are as defined above and
wherein the counter ions A⁻ of this group are preferably mono- to pentacontavalent, more preferably mono- to decavalent, even more preferably mono- to pentavalent, most preferably pentavalent, tetravalent, trivalent, divalent or monovalent anions,
or the counter anions are selected from the group consisting of carboxylate anions based on poly (acrylic acid) homo and copolymers and poly (itaconic acid) homo and copolymers, wherein the anions of this group are preferably di- to 30000-valent, more preferably di-to kiliavalent, even more preferably deca- to kiliavalent, even further preferably pentaconta- to kiliavalent, and most preferably hecta- to kiliavalent anions, and preferably from polymeric fatty acid carboxylates of the type

—O—C(O)—R⁶(—X—C(O)—R⁶)ₘ₋₁—X—C(O)—R⁷

being single chain molecules without esterified OH-substituents, or preferably from polymeric fatty acid carboxylates of the type

R¹[(—C(O)—X—R₆)ₘ—C(O)O⁻]ₓ as defined above

being branched or dendrimeric (self repeating) motifs-containing carboxylates, in particular derived from 2,2′-di-hydroxymethyl propanoic acid.

The synthesis of dendrimeric structures of 2,2′-di-hydroxymethyl propanoic acid is described in US 2016/0102179.

In a further preferred embodiment according to the invention, the counter ions A⁻ of the compounds according to the invention of the general formula (I) as defined above are mono-to trivalent inorganic anions and mono- to 30000-valent, preferably mono- to kiliavalent organic anions selected from the group consisting of halide anions, such as chloride, bromide, iodide, sulphate, phosphate, phosphonate, sulphonate, methosulphate, carboxylate anions, such as acetate, propionate, lactate, octanoate, 2-ethyl-hexanoate, dodecanoate, hexadecanoate, octadecanoate, oleate, ricinoleate, 12-hydroxy-octadecanoate, succinate, maleate, tartrate, polyethercarboxylate, polymeric fatty acid carboxylates of the type

R¹[(—C(O)—X—R⁶)ₘ—C(O)—X—R⁷]ₓ or

R¹[(X—C(O)—R⁶)ₘ—X—C(O)—R⁷]ₓ,

wherein either R¹ or at least one of R⁷, or both R¹ and at least one of R⁷ bear one or more carboxylate groups, preferably with X=O, in particular linear polymeric fatty acid carboxylates of the type

—O—C(O)—R⁶(—X—C(O)—R⁶)ₘ₋₁—X—C(O)—R⁷, preferably

—O—C(O)—R⁶—(O—C(O)—R⁶)ₘ—O—C(O)—R⁷,

branched linear polymeric fatty acid carboxylates,
i.e. derived from branched poly fatty acid structures, in particular branched linear polymeric fatty acid carboxylates derived from partial esters of polyfunctional carboxylic acids, in particular of the dicarboxylic acids succinic acid and maleic acid, with castor oil or lesquerella oil, such as

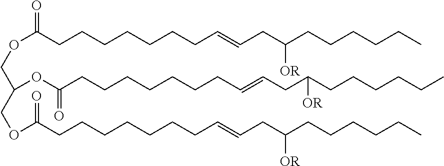

with one

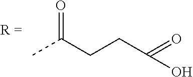

and the remaining two

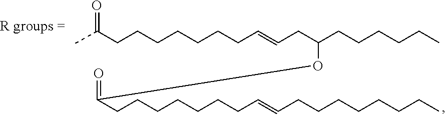

dendritic polymeric fatty acid carboxylates,
i.e. derived from dendritic poly fatty acid structures,
or of the types

X—R⁶(—C(O)—X—R⁶)ₘ₋₁—C(O)—X—R⁷ or

R⁶(—C(O)—X—R⁶)ₘ₋₁—C(O)—X—R⁷,

wherein in the two latter types the R⁷ group bears at least one anionic carboxylate group, or of the type

R¹[(—C(O)—X—R₆)ₘ—C(O)O⁻]ₓ, such as

wherein X, R¹, R⁶, R⁷, m and x are as defined above and wherein the counter ions A⁻ of this group are preferably mono- to pentacontavalent, more preferably mono- to decavalent, even more preferably mono- to pentavalent, most preferably pentavalent, tetravalent, trivalent, divalent or monovalent anions,
or the group consisting of poly (acrylic acid) homo and copolymers, poly (itaconic acid) homo and copolymers, wherein the anions of this group are preferably di- to 30000-valent, more preferably di-to kiliavalent, even more preferably deca- to kiliavalent, even further preferably pentaconta- to kiliavalent, and most preferably hecta- to kiliavalent anions, preferably from polymeric fatty acid carboxylates of the type

—O—C(O)—R⁶(—X—C(O)—R⁶)ₘ₋₁—X—C(O)—R⁷

being single chain molecules without esterified OH-substituents, or preferably from polymeric fatty acid carboxylates of the type

R¹[(—C(O)—X—R₆)ₘ—C(O)O⁻]ₓ

as defined above
being branched or dendrimeric (self repeating) motifs-containing carboxylates, in particular derived from 2,2′-di-hydroxymethyl propanoic acid.

The desired counter ion can be incorporated into the quaternized materials either in the course of the quaternization or by anion exchange. In this context it is possible to exchange inorganic counter ions, such as chlorine or bromine against organic counter ions, such as fatty acid carboxylates or polymeric fatty acid carboxylates by adding alkali salts, preferred sodium and potassium salts, of fatty acids or polymeric fatty acids to the initially inorganic counter ions containing materials thus yielding the target materials and alkali metal halides, in particular NaCl, NaBr, KCl, KBr.

In a preferred embodiment according to the invention, in Formula (III) and/or (IV), X=O, and preferably the compound according to the invention does not contain any amide group.

Amide bonds are generally more stable to hydrolysis, however, they also confer structural rigidity when compared to an ester group. According to this embodiment, the group X thus represents an oxygen atom in in all structures of the Formula (III) and/or (IV) present in the compound, and it is preferred that the compound does not contain any amide group at all.

In another preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—]₂,

wherein R^{1*} is a divalent C1-C100 hydrocarbon radical, preferably a C1-C12 alkylene, most preferably a methylene, ethylene, 1,3-propylene, 1,4-butylene, 1,6-hexylene, 1,2-propylene, 1,3-butylene radical,
m is independently selected from 1 to 12, and
R⁶ is as defined above.

Therein, the moieties of the formula

R^{1*}[(—O—C(O)—R⁶)ₘ—O—C(O)—]₂,

are preferably formed starting from alkylene diols, more preferably from α,o-alkylene diols such as 1,2-ethane diol, 1,3-propane diol, 1,4-butane diol and 1,6 hexanediol, by sequential or blockwise ester chain formation.

Regardless whether single hydroxy-substituted carboxylic acids are added in an iterative manner or if estolide chains with a carboxylic acid group are brought to reaction with a such diol, using an excess of the carboxylic acid reactant results in the formation of a product in which the diol is predominantly esterified in the same manner on both ends, i.e. a symmetrical structure of the formula

R^{1*}[(—O—C(O)—R⁶)ₘ—O—C(O)—]₂

is thus obtained.

The structure of the group R^{1*} thus usually corresponds directly to the alkylene diols applied as starting materials.

According to the invention, R^{1*} is divalent C2-C100 hydrocarbon radical, which includes all types of linear, branched and cyclic aliphatic and aromatic divalent hydrocarbon groups, such as alkylenes, alkenylenes, alkynylenes as well as aromatic structures, such as phenylenes.

As C1-C12 alkylene diols are preferred starting materials, R^{1*} is preferably a C1-12 alkylene group, more preferably a methylene, ethylene, n-propylene, n-butylene, n-pentylene, n-hexylene, even more preferably a methylene, ethylene, n-propylene or n-butylene or n-hexylene group.

While according to this embodiment m is independently selected, it is preferred that both m of the general structure

R^{1*}[(—O—C(O)—R⁶)ₘ—O—C(O)—]₂,

are the same, as the moiety is typically symmetrical.

It is further preferred that m is independently selected from 1-6, more preferably from 1-4, even more preferably both m are the same and selected from 1-6, most preferably both m are the same and selected from 1-4.

According to the embodiment, R⁶ is as defined above, but preferably the R⁶ radical is selected from linear alkylene groups and linear alkenylene groups, in particular from linear C6-C24 alkyene groups such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

More preferably, R⁶ is derived from C7-C25 fatty acids bearing one hydroxyl group as substituent, even more preferably R⁶ is derived from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid.

Most preferably R⁶ is derived from ricinoleic acid.

It is generally preferred in this and any other embodiment as described herein that all R⁶ groups of a moiety of the general formula

R^{1*}[(—O—C(O)—R⁶)ₘ—O—C(O)—]₂,

are the same.

In a further preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—]₂,

wherein R^{1*} is selected from methylene, ethylene, 1,3-propylene, 1,4-butylene, 1,6-hexylene, 1,2-propylene, 1,3-butylene,
R⁶ is derived from C8-C24 monocarboxy-monohydroxy carboxylic acids, in particular from ricinoleic acid, 12-hydroxy stearic acid, lesquerolic acid, 11-hydroxy-undecanoic acid, and m is independently selected from 1 to 6.

According to this embodiment, it is preferred that all R⁶ are derived from the same carboxylic acid, and that both m of the structure are the same.

In an even further preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—]₂,

which is represented by one of the following structural formulas: —C(O)—O—(mono or oligo C8-C24 hydroxy fatty acid)—C(O)—O—(C2-C10 hydrocarbon)—O—C(O)— (mono or oligo C8-C24 hydroxy fatty acid)—O—C(O)— wherein
C2-C10 hydrocarbon is a C2-C10 hydrocarbylene group, in particular derived from ethylene glycol, 1,3 propylene glycol, 1,4 butanediol, 1,6 hexanediol, 1,2 propylene glycol, 1,3 butanediol,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid, with a degree of oligomerization of 2 to 20, preferred, 2 to 10, more preferred 2 to 6, even more preferred 2 to 4.

Such compounds are exemplified by the following structural formulas:

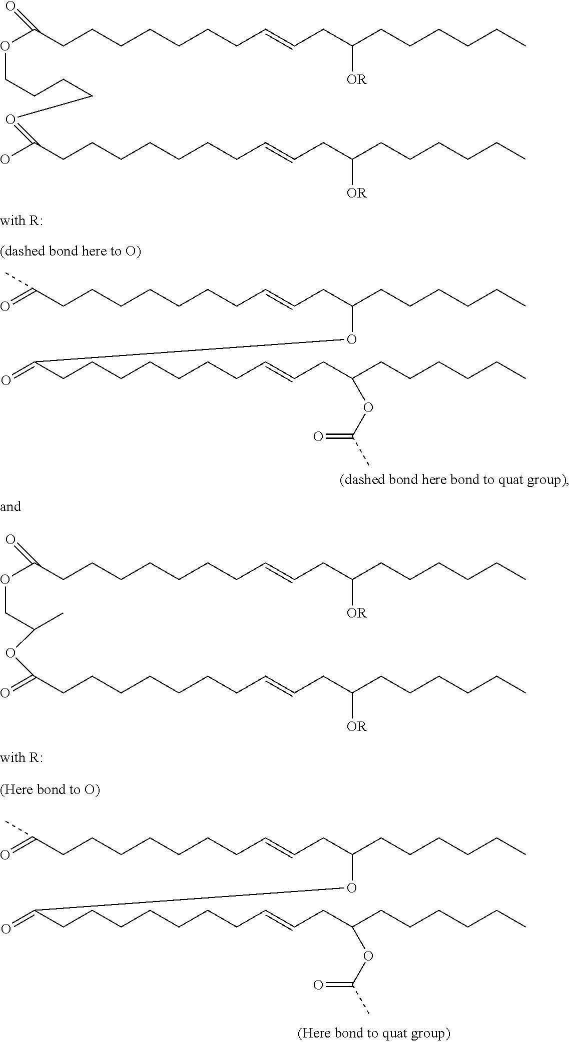

In still another further preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—R⁷*—]₂,

wherein
R^{1*}, R⁶, and m are as defined above,
and R^{7*}is a C1-C12 alkylene group, preferably a methylene, ethylene, propylene or butylene group.

In this embodiment, the estolide chains of the above-shown moieties are bonded to alkylene groups, as typically, after the formation of the estolide chain structures comprised by the moieties of the general formula

R^{1*}[(—O—C(O)—R⁶)ₘ—O—C(O)—R⁷*—]₂,

the terminal hydroxy groups of the chain structures are brought to reaction with carboxylic acids or carboxylic acid chlorides having a functionalized alkyl chain, in particular halo-alkyl carboxylic acid chlorides. By this, the precursor of the group R^{7*} is attached to the structure, and upon further functionalization the moiety of the general structure

R^{1*}[(—O—C(O)—R⁶)ₘ—O—C(O)—R⁷*—]₂

is obtained.

According to this embodiment, R^{7*} is a C1-C12 alkylene group, preferably a methylene, ethylene, propylene or butylene group, most preferably a methylene group.

For instance, R^{7*} being a methylene group may be obtained by esterification of the terminal hydroxy groups of the estolide chains with chloro-acetic acid chloride, and subsequent functionalization, e.g. by using the chloro group as a leaving group, for instance in a quaternization reaction of tertiary amines.

In a further preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—R⁷*—]₂,

wherein
R^{1*} is selected from methylene, ethylene, 1,3-propylene and 1,4-butylene, 1,6-hexylene,
R⁶ is derived from C8-C24 monocarboxy-monohydroxy carboxylic acids, in particular ricinoleic acid, 12-hydroxy stearic acid, lesquerolic acid, 11-hydroxy-undecanoic acid,
m is independently selected from 1 to 6,
and R^{7*} is selected from methylene and ethylene.

In an even further preferred embodiment, in the compound according to the invention at least one of the groups R¹, R², R³, R⁴, R⁵ present in the cationic structure of the general formulas (I) and (II) contains at least one moiety of the formula

R^{1*}[(—O—C(O)—R⁶)ₘ—O—C(O)—R⁷*—]₂

which is represented by one of the following structural formulas:
i) —CH₂—C(O)—O—(mono or oligo C8-C24 hydroxy fatty acid)—C(O)—O—(C2-C10 hydrocarbon)—O—C(O)—(mono or oligo C8-C24 hydroxy fatty acid)—O—C(O)—CH₂—
or
   ii) —CH₂CH₂—C(O)—O—(mono or oligo C8-C24 hydroxy fatty acid)—C(O)—O—(C2-C10 hydrocarbon)-O—C(O)—(mono or oligo C8-C24 hydroxy fatty acid)—O—C(O)—CH₂CH₂—, wherein
C2-C10 hydrocarbon is a C2-C10 hydrocarbylene group, in particular derived from ethylene glycol, 1,3 propylene glycol, 1,4 butanediol, 1,6 hexanediol, 1,2 propylene glycol, 1,3 butanediol,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid, with a degree of oligomerization of 2 to 20, preferred, 2 to 10, more preferred 2 to 6, even more preferred 2 to 4.

Such compounds are exemplified by the following structural formulas:

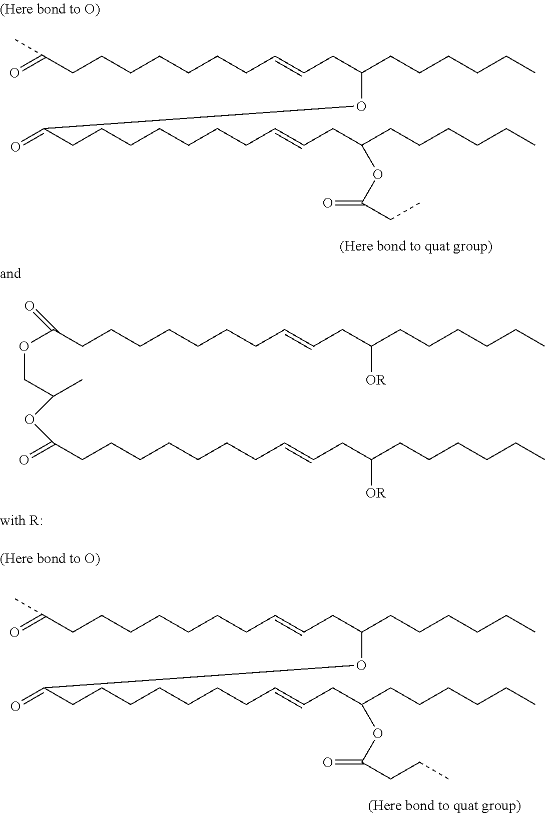

In a further preferred embodiment, in the compound according to the invention at least one moiety of the general formula

R^{1*}[(—O—C(O)—R⁶)ₘ—O—C(O)—R⁷*—]₂

is bonded to a quaternary N atom on one or both terminal R^{7*} groups.

Accordingly, according to this embodiment, the compound according to the invention contains at least one moiety of the general formula

R^{1*}[(—O—C(O)—R⁶)ₘ—O—C(O)—R⁷*—N⁺]₂,

wherein both terminal R^{7*} groups are bonded to a quaternary N atom, and/or at least one moiety of the general formula

N⁺—R⁷*C(O)—O—(R⁶—C(O)—O)ₘ—R^{1*}—^{*}(—O—C(O)—R⁶)ₘ—O—C(O)—R⁷*—,

wherein one terminal R^{7*} group is bonded to a quaternary N atom.

It is further preferred that the one or two quaternary N atom each bear two groups independently selected from methyl, ethyl, propyl and butyl groups.

More preferably, the one or two quaternary N atoms each bear two methyl substituents, and even more preferably, the fourth substituent is either an alkyl amino group, or an alkyl group substituted by an ammonium group, most preferably the fourth substituent is selected from an ethylene dimethylammonium group, a propylene dimethylammonium group, a butylene dimethylammonium group or a hexylene dimethylammonium group.

Preferably, these groups are derived from N,N,N′,N′-tetramethyl-1,2-ethylenediamine, N,N,N′,N′-tetramethyl-1,4-butylenediamine, N,N,N′,N′-tetramethyl-1,6-hexylenediamine. Alternatively, quaternary ammonium groups bearing one methyl group can be derived from N,N′-dimethylpiperazine.

In another preferred embodiment according to the invention, at least one moiety of the general formula

R^{1*}[(—O—C(O)—R⁶)ₘ—O—C(O)—R⁷*—]₂

is bonded to a quaternary N atom on one or both terminal R^{7*} groups, and the compound is a di-quat or a tetra-quat compound.

Therein, it is preferred that both terminal R^{7*} groups are bonded to a quaternary N atom, more preferably both terminal R^{7*} groups are each bonded to a quaternary N atom bearing three alkyl substituents each having 1 to 12 carbon atoms, or to a quaternary N atom represented by the formula —N⁺(CH₃)₂-ALK-N⁺(CH₃)₃, wherein ALK is a divalent alkylene group having 1-12 carbon atoms, preferably a linear alkylene group.

According to the embodiment, it is preferred that if both terminal R^{7*} groups are bonded to a quaternary N atom bearing three alkyl substituents each having 1 to 12 carbon atoms, it is preferred that the alkyl substituents are selected from methyl, ethyl, propyl and butyl groups, most preferably all three substituents are methyl groups.

If both terminal R^{7*} groups are bonded to a quaternary N atom represented by the formula —N⁺(CH₃)₂-ALK-N⁺(CH₃)₃ as defined above, it is preferred that the group ALK is a methylene group, ethylene group, n-propyl group, n-butylene group or n-hexylene group.

In a further preferred embodiment, the compound according to the invention comprises at least two moieties of the general formula

R^{1*}[(—O—C(O)—R⁶)ₘ—O—C(O)—R⁷*—]₂,

wherein said moieties are linked to each other via di-quaternary ammonium alkylene groups of the general structure

—N⁺(CH₃)₂-ALK-N⁺(CH₃)₂—,

wherein ALK is a divalent alkylene group having 1-12 carbon atoms, preferably a linear alkylene group.

Preferably, the compound comprises more than 4, more preferably more than 6, even more preferably more than 8 moieties of the formula

R^{1*}[(—O—C(O)—R⁶)ₘ—O—C(O)—R⁷*—]₂

which are linked via diammonium alkylene groups.
Further preferably, the group ALK is independently selected from ethylene, n-propylene, n-butylene or n-hexylene, and more preferably all groups ALK are the same type of alkylene group.

In another preferred embodiment of the compund according to the invention, at least one of the groups R¹, R², R³, R⁴, R⁵ present in the cationic structure of the general formulas (I) and (II) contains at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—

wherein R⁶ is as defined above,
l is an integer independently selected from 0-20, more preferably from 1-12, even more preferably from 2 to 10, and
L is a divalent hydrocarbon radical which may have 1 to 30 carbon atoms and may contain optionally one or more groups selected from —O—, —S—, —NH—, —C(O)—, —C(S)—, and tertiary
amino groups

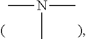

preferably L is a divalent alkylene or alkenylene radical having 1 to 30 carbon atoms, more preferably L is selected from methylene, ethylene, propylene, butylene, pentylene, hexylene, heptylene, octylene, nonylene, ethenylene, propenylene, butenylene, pentenylene, hexenylene, heptenylene, octenylene, nonenylene, most preferably L is selected from methylene, ethylene, ethenylene or butenylene.

According to this embodiment, R⁶ is preferably independently derived from C8-C24 monocarboxy-monohydroxy carboxylic acids, in particular from ricinoleic acid, 12-hydroxy stearic acid, lesquerolic acid, and 11-hydroxy-undecanoic acid.

In a preferred embodiment of the compound according to the invention,

at least one of the groups R¹, R², R³, R⁴, R⁵ present in the cationic structure of the general formulas (I) and (II) contains at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—,
wherein L and l are as defined above,
   and R⁶ is independently derived from C8-C24 monocarboxy-monohydroxy carboxylic acids, preferably derived from. ricinoleic acid, 12-hydroxy stearic acid, lesquerolic acid, 11-hydroxy-undecanoic acid, most preferably R⁶ is derived from ricinoleic acid.

Further preferably, L is selected from methylene or ethylene, —CH═CH— and —C(═CH₂)—CH₂—, l is independently selected from an integer in the range of 0 to 6, and R⁶ is derived from ricinoleic acid.

In a preferred embodiment of the compound according to the invention, at least one of the groups R¹, R², R³, R⁴, R⁵ present in the cationic structure of the general formulas (I) and (II) contains at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—,

wherein L is selected from methylene, ethylene, and ethenylene,
R⁶ is derived from ricinoleic acid, and
l is independently selected from 0, 1, 2 and 3, and the sum of l is in the range of 0-4.

Preferably, L is an ethylene group, R⁶ is derived from ricinoleic acid, and l is independently selected from 0 or 1.

In a preferred embodiment of the compound according to the invention, at least one of the groups R¹, R², R³, R⁴, R⁵ present in the cationic structure of the general formulas (I) and (II) contains at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—,

which is represented by the following structure:
—O—C(O)—(mono or oligo C8-C24 hydroxy fatty acid)—O—C(O)—(C1-C12 hydrocarbon)—C(O)—O— (mono or oligo C8-C24 hydroxy fatty acid)—C(O)—O—
wherein
C1-C12 hydrocarbon is a C1-C12 hydrocarbylene group, preferably a C2 to C10 hydrocarbylene group, and
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, with a degree of oligomerization of 2 to 20, preferred, 2 to 10, more preferred 2 to 6, even more preferred 2 to 4.

The C1-C12 hydrocarbon group is preferably derived from succinic acid, maleic acid, itaconic acid, adipic acid, sebacic acid, or dodecanedioic acid, the mono or oligo C8-C24 hydroxy fatty acid group is preferably derived from mono or oligo ricinoleic acid with a degree of oligomerization of 2 to 20, preferred, 2 to 10, more preferred 2 to 6, even more preferred 2 to 4.

Compounds of such structure are exemplified by

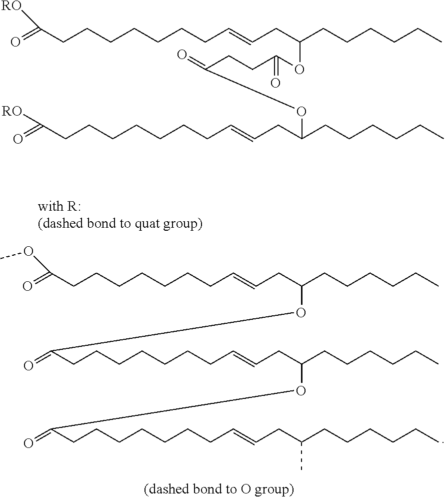

In a preferred embodiment of the compound according to the invention, at least one of the groups R¹, R², R³, R⁴, R⁵ present in the cationic structure of the general formulas (I) and (II) contains at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—R¹²,

wherein L, l, R⁶ are as defined above,
and R¹² is a C1 to C12 linear or branched hydrocarbylene group which may contain up to 4 —O— groups and up to 4 tertiary amino groups, and which is bonded to the —O— group of an ester group on one terminus and to a quaternary N atom at the other terminus,
preferably R¹² is derived from tertiary amino alcohols, in particular from the amino alcohols having the structures

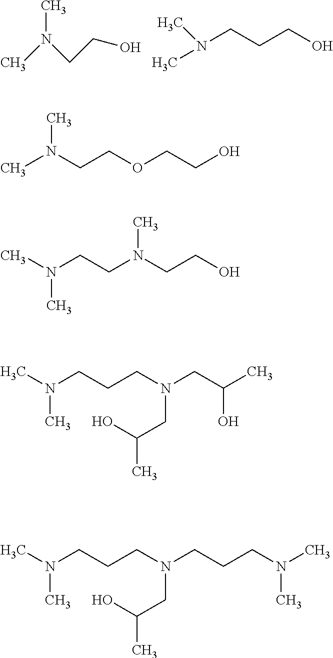

Preferably, L, l and R⁶ are as defined above, and R¹² is selected from —CH₂CH₂— and —CH₂CH₂CH₂—.

Further preferably, l is independently selected from the range of 0-6, preferably 1-6, more preferably 2-6.

R⁶ is preferably derived from ricinoleic acid, and
R¹² is preferably selected from —CH₂CH₂— and —CH₂CH₂CH₂—.

The present invention also relates to a process for the synthesis of compounds of the general formula (I)

R¹(—F)ₓ (I)

as defined by all of the previous embodiments according to the invention, wherein
alkyl halogenides are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa),

or
esters of halogen carboxylic acids, preferably chloro acetic acid, with alcohols or epoxides, as defined above, are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa),

or
epoxy functionalized ethers and esters, preferably glycidyl ethers and esters, with alcohols or carboxylic acids, as defined above, are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa)

in the presence of an acid,
or
tertiary amino groups containing hydrocarbons are reacted with esters of halogen carboxylic acids, as defined above, containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa),

or
tertiary amino groups containing hydrocarbons are reacted with epoxy functionalized ethers and esters, as defined above, containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa)

in the presence of an acid,
wherein X, R⁶, R⁷, m and x are as defined above.

According to this embodiment, it is preferred when

### X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl,
and m is 1,2, 3, 4 or 5.

It is most preferred according to this embodiment when

### X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid,
and m is 1,2, 3, 4 or 5.

In a preferred embodiment according to the invention, a process for the synthesis of compounds of the general formula (I)

R¹(—F)ₓ (I)

with R¹ being linked through a quaternized nitrogen atom N⁺ to R³, R⁴, and R⁵, and R¹(—F)ₓ containing at least one moiety of the general formula (III)

(—X—C(O)—R⁶)ₘ—X—C(O)— (III),

or of the general formula (IV)

(—C(O)—X—R⁶)ₘ—C(O)—X— (IV),

preferably
bearing at least one moiety of the general formula (IIIa)

(—X—C(O)—R⁶)ₘ—X—C(O)—R⁷ (IIIa),

or of the general formula (IVa)

(—C(O)—X—R⁶)ₘ—C(O)—X—R⁷ (IVa)

is provided,
wherein
alkyl halogenides are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷(IVa),

or
esters of halogen carboxylic acids, preferably chloro acetic acid, with alcohols or epoxides, as defined above, are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa),

or
epoxy functionalized ethers and esters, preferably glycidyl ethers and esters, with alcohols or carboxylic acids, as defined above, are reacted with tertiary amines containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa)

in the presence of an acid,
or
tertiary amino groups containing hydrocarbons are reacted with esters of halogen carboxylic acids, as defined above, containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa),

or
tertiary amino groups containing hydrocarbons are reacted with epoxy functionalized ethers and esters, as defined above, containing at least one moiety

(—X—C(O)—R⁶)ₘ—X—C(O)— (III) or

(—C(O)—X—R⁶)ₘ—C(O)—X— (IV),

preferably bearing at least one moiety

(—X—C(O)—R⁶)ₘ—X—C(O)—R⁷ (IIIa) or

(—C(O)—X—R⁶)ₘ—C(O)—X—R⁷ (IVa)

in the presence of an acid
wherein X, R⁶, R⁷, m and x are as defined above.

According to this embodiment, it is preferred when

### X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl,
and m is 1,2, 3, 4 or 5.

It is most preferred according to this embodiment when

### X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid, and m is 1,2, 3, 4 or 5.

In another preferred embodiment of the invention, a process for the synthesis of compounds of the general formula (I)

R¹(—F)ₓ (I)

with R¹ being linked to a quaternized nitrogen atom N⁺ and R¹(—F)ₓ bearing at least one moiety of the general formulas (XIII), (XIV), (XIIIa) or (XIVa)

—R¹⁰(—X—C(O)—R⁶)ₘ—X—C(O)— (XIII)

—R¹⁰(—C(O)—X—R⁶)ₘ—C(O)—X— (XIV),

preferably

—R¹⁰(—X—C(O)—R⁶)ₘ—X—C(O)—R⁷ (XIIIa)

—R¹⁰(—C(O)—X—R⁶)ₘ—C(O)—X—R⁷ (XIVa)

is provided, wherein
alkyl halogenides are reacted with tertiary amines bearing at least one moiety of the general formulas (XIII), (XIV), (XIIIa) or (XIVa) in order to obtain such compounds wherein R¹ is linked to a quaternized nitrogen atom N⁺ and R¹(—F)ₓ is bearing at least one moiety of the general formulas (XIII), (XIV), (XIIIa) or (XIVa),
or
esters of halogen carboxylic acids, preferred chloro acetic acid, the esters being formed with alcohols or epoxides, are reacted with tertiary amines bearing at least one moiety of the general formulas (XIII), (XIV), (XIIIa) or (XIVa) in order to obtain such compounds as stated above,
or
epoxy functionalized ethers and esters, preferred glycidyl ethers and esters, formed from alcohols or carboxylic acids, as defined above, are reacted with tertiary amines bearing at least one moiety of the general formulas (XIII), (XIV), (XIIIa) or (XIVa) in the presence of an acid in order to obtain such compounds as stated above,
or
tertiary amino groups containing hydrocarbons are reacted with esters of halogenated carboxylic acids bearing at least one moiety of the general formulas (XIII), (XIV), (XIIIa) or (XIVa) in order to obtain such compounds as stated above, or
tertiary amino groups containing hydrocarbons are reacted with epoxy functionalized ethers and esters, as defined above, bearing at least one moiety of the general formulas (XIII), (XIV), (XIIIa) or (XIVa) in the presence of an acid, wherein R¹⁰, X, R⁶, R⁷, m and x are as defined above in order to obtain such compounds as stated above.

According to this embodiment, it is preferred when

### X=O,

R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom, and if present,
R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom,
R¹ is an unsubstituted C1-C8 alkylene group not containing functional group, or R¹ is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units, and
m is 1-10, preferably 1, 2, 3, 4 or 5.

It is even more preferred when X=O,

R⁶ is selected from hexadecylene, heptadecylene, octadecylene, nonadecylene, eicosylene, hexadecenylene, heptadecenylene, octadecenylene, nonadecenylene, eicosenylene, and if present,
R⁷ is selected from hexadecyl, heptadecyl, octadecyl, nonadecyl, eicosyl hexadecenyl, heptadecenyl, octadecenyl, nonadecenyl, eicosenyl,
R¹ is an unsubstituted C1-C8 alkylene group not containing functional group, or R¹ is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units,
and m is 1,2, 3, 4 or 5.

It is most preferred according to this embodiment when X=O,

R⁶ is derived from ricinoleic acid, 10-hydroxy stearic acid, 12-hydroxy stearic acid or lesquerolic acid, and if present,
R⁷ is derived from oleic acid, ricinoleic acid or stearic acid,
R¹ is an unsubstituted C1-C8 alkylene group not containing functional group, or R1 is a linear C3 to C50 alkylene group derived from diglycidyl ether, glycerol diglycidyl ether, diglycerol diglycidyl ether, diethylene glycol diglycidyl ether,
and m is 1, 2, 3, 4 or 5.

The invention further relates to the use of the above-described polymeric fatty acid compounds of the general formula (I) in cosmetic formulations for skin and hair care, in particular conditioners and shampoos, in polishing agents for treating and coating hard surfaces, in formulations for drying automobiles and other hard surfaces, for example following automatic washing, for finishing textiles and textile fibers, as separate softeners for use after textiles have been washed with nonionic or anionic/nonionic detergent formulations, as softeners in formulations for washing textiles that are based upon nonionic or anionic/nonionic surfactants, and as means for preventing or removing wrinkles in textiles.

The invention further relates to the use of the above-described polymeric fatty acid compounds in cosmetic compositions for the treatment of fibers, preferred amino acid based fibers, more preferred human hair, in particular being useful for strengthening of hair, for hair color retention, for hair shine enhancement, for hair color enhancement, for hair color protection, for shaping of hair, in particular the curling and straightening of hair, for hair conditioning, for hair smoothening or softening, for improving manageability of the hair, in particular for improving the combability of the hair, the anti-frizz and anti-static properties.

Preferred compositions according to the invention are cosmetic compositions for the treatment of hair selected from the group consisting of a hair shampoo composition, hair care composition, hair conditioning composition, hair strengthening composition, hair coloration or dyeing composition, hair combability improving composition, anti-frizz composition, hair rinse-off and leave-on compositions.

The invention further relates to compositions that contain at least one of the polymeric fatty acid compounds, together with at least one additional component that is commonly used in such a composition.

Below, a number of typical examples of these types of compositions are provided, in which the polymeric fatty acid compounds of the invention may be advantageously used. Typical adjuvants in these types of compositions are, e.g., those materials described in A. Domsch: Die kosmetischen Praeparate [Cosmetic Preparations] Vol. I and II, 4th Edition, Verl. fuer chem. Industrie [Publishers for the Chemical Industry], U. Ziolkowsky K G, Augsburg, and the International Cosmetic Ingredient Dictionary and Handbook 7^{th} Ed. 1997 by J. A. Wenninger, G. N. McEwen Vol. 1-4 by The Cosmetic, Toiletry and Fragrance Association Washington D.C.

In particular, the invention relates to such compositions as defined above for the treatment of hair selected from the group consisting of hair shampoo compositions, hair conditioning compositions, hair strengthening compositions, hair coloration or dyeing compositions, hair combability improving compositions, anti-frizz compositions, hair rinse-off and leave-on compositions. In the subsequent formulations, the term “Polymeric fatty acid compound of the invention” is used to refer to the compounds as defined above.

### Formulation Examples

### Anionic Shampoo

This formulation example is intended as a basic formulation. Anionic shampoos customarily contain, but are not limited to, the following components: Alkyl sulfates, alkyl ether sulfates, sodium lauryl sulfate, sodium lauryl ether sulfate, ammonium lauryl sulfate, ammonium lauryl-ether sulfate, TEA-lauryl sulfate, TEA-lauryl-ether sulfate, alkylbenzene sulfonates, α-olefinsulfonates, paraffin sulfonates, sulfosuccinates, N-acyltaurides, sulfate-glycerides, sulfatized alkanolamides, carboxylate salts, N-acyl-amino acid salts, silicones, etc.

| Components | wt-% |
|---|---|
| Ammonium lauryl sulphate | 10.00-30.00 |
| Ammonium lauryl-ether sulphate | 5.00-20.00 |
| Cocamidopropyl betaine | 0.00-15.00 |
| Lauramide DEA | 0.00-5.00 |
| Cocamide MEA | 0.00-5.00 |
| Dimethicone copolyol | 0.00-5.00 |
| (dimethylsiloxane glycol copolymer) | |
| Cyclopentasiloxane | 0.00-5.00 |
| Polymeric fatty acid | 0.50-5.00 |
| compound of the invention | |
| Polyquaternium-10 | 0.00-2.00 |
| Preservatives | 0.00-0.50 |
| Fragrance | 0.00-5.00 |
| Deionized water | q.s. 100% |
| Sodium chloride | q.s. |

### Nonionic Shampoo

This formulation example is intended as a basic formulation. Nonionic shampoos customarily contain, but are not limited to, the following components: monoalkanolamides, monoethanolamides, monoisopropanolamides, polyhydroxy derivatives, sucrose monolaurate, polyglycerine ether, amine oxides, polyethoxylated derivatives, sorbitol derivatives, silicones, etc.

| Components | wt-% |
|---|---|
| Lauramide DEA | 10.00-30.00 |
| Lauramide oxide | 5.00-20.00 |
| Cocamide Mea | 0.00-5.00 |
| Dimethicone copolyol | 0.00-5.00 |
| Polymeric fatty acid | 0.50-5.00 |
| compound of the invention | |
| Preservatives | 0.00-0.50 |
| Fragrance | 0.00-5.00 |
| Deionized water | q.s. 100% |
| Sodium chloride | q.s. |

### Amphoteric Shampoo

This formulation example is intended as a basic formulation. Formulations of this category customarily contain, but are not limited to, the following components: N-alkyl-iminodipropionates, N-alkyl-iminopropionates, amino acids, amino acid derivatives, amido betaine, imidazolinium derivatives, sulfobetaines, sultaines, betaines, silicones, etc.

| Components | wt-% |
|---|---|
| PEG-80-sorbitane laurate | 10.00-30.00 |
| Lauroamphoglycinate | 0.00-10.00 |
| Cocamidopropyl-hydroxysultain | 0.00-15.00 |
| PEG-150-distearate | 0.00-5.00 |
| Laurylether-13-carboxylate | 0.00-5.00 |
| Polymeric fatty acid | 0.50-5.00 |
| compound of the invention | |
| Fragrance | 0.00-5.00 |
| Deionized water | q.s. 100% |
| Sodium chloride | q.s. |

### Cationic Shampoo

This formulation example is intended only as a basic formulation. Formulations of this category customarily contain, but are not limited to, the following components: bis-quaternary ammonium compounds, bis-(trialkylammonium acetyl)diamines, amido amines, ammonium alkyl esters, silicones, etc.

| Components | wt-% |
|---|---|
| Laurylether-13-carboxylate | 10.00-30.00 |
| Isopropyl myristate | 5.00-20.00 |
| Cocamidopropyl-betaine | 0.00-15.00 |
| Lauramide DEA | 0.00-5.00 |
| Cocamide MEA | 0.00-5.00 |
| Polymeric fatty acid | 0.50-5.00 |
| compound of the invention | |
| Preservatives | 0.00-0.50 |
| Fragrance | 0.00-5.00 |
| Deionized water | q.s. 100% |
| Sodium chloride | q.s. |

### Setting Agents

This formulation example is intended only as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickeners, silicones, etc.

| Components | wt-% |
|---|---|
| Ceteareth-20 | 0.10-10.00 |
| Steareth-20 | 0.10-10.00 |
| Stearyl alcohol | 0.10-10.00 |
| Stearamidopropyl-dimethylamine | 0.00-10.00 |
| Dicetyldimonium-chloride | 0.00-10.00 |
| Polymeric fatty acid | 0.50-5.00 |
| compound of the invention | |
| Cyclopentasiloxane | 0.00-5.00 |
| Dimethicone | 0.00-5.00 |
| Preservatives | 0.00-0.50 |
| Fragrance | 0.00-5.00 |
| Deionized water | q.s. 100% |

### “Clear Rinse-Off” Setting Agents

This formulation example is intended as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, etc.

| Components | wt-% |
|---|---|
| Glycerin | 0.10-10.00 |
| Cetrimonium chloride | 0.00-10.00 |
| Polymeric fatty acid | 0.50-5.00 |
| compound of the invention | |
| Hydroxyethyl cellulose | 0.00-5.00 |
| Preservatives | 0.00-0.50 |
| Fragrance | 0.00-5.00 |
| Deionized water | q.s. 100% |

### Foam Setting Agents for Hair

This formulation example is intended as a basic formulation. Formulations of this category contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorated aerosol propellants, dimethylether, compressed gases, etc.

| Components | wt-% |
|---|---|
| Polymeric fatty acid | 0.50-5.00 |
| compound of the invention | |
| Nonoxynol-15 | 0.00-2.00 |
| Nonoxynol-20 | 0.00-2.00 |
| Aerosol propellants | 0.00-20.00 |
| Preservatives | 0.00-0.50 |
| Fragrance | 0.00-5.00 |
| Deionized water | q.s. 100% |

### Pump Spray (Setting Agents) for Hair

This formulation example is intended only as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, etc.

| Components | wt-% |
|---|---|
| Polymeric fatty acid | 0.50-5.00 |
| compound of the invention | |
| Cyclomethicone | 0.00-80.00 |
| Ethanol | 0.00-80.00 |
| Preservatives | 0.00-0.50 |
| Fragrance | 0.00-5.00 |
| Deionized water | q.s. 100% |

### Setting Agent Spray for Hair

This formulation example is intended as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethyl ether, compressed gases, etc.

| Components | wt-% |
|---|---|
| Polymeric fatty acid | 0.50-5.00 |
| compound of the invention | |
| Cyclomethicone | 0.00-80.00 |
| Ethanol | 0.00-50.00 |
| Aerosol propellants | 0.00-50.00 |
| Preservatives | 0.00-0.50 |
| Fragrance | 0.00-5.00 |
| Deionized water | q.s. 100% |

### Gel Setting Agents for Hair

This formulation example is intended as a basic formulation. Formulations of this category customarily contain, but are not limited to, the following components: thickening agents, cellulose derivatives, acrylic acid derivatives, fixative polymers, conditioning chemicals, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, silicones, solvents, ethanol, isopropanol, isoparaffin solvents, etc.

| Components | wt-% |
|---|---|
| Polymeric fatty acid | 0.50-5.00 |
| compound of the invention | |
| Hydroxyethyl cellulose | 0.00-2.00 |
| Citric acid | 0.00-2.00 |
| Preservatives | 0.00-0.50 |
| Fragrance | 0.00-5.00 |
| Deionized water | q.s. 100% |

### Rinse Off Conditioner

This formulation example is intended as a basic formulation. Formulations of this category customarily contain, but are not limited to, the following components: hydrocarbon based cationic conditioning agents, silicone based cationic conditioning agents, high melting fatty compounds, low melting oil like ester compounds, thickening agents, cellulose derivatives, fixative polymers, ethylene glycols, propylene glycols, glycol esters, glycerin, glycerin esters, monohydric alcohols, polyhydric alcohols, cationic polymers, nonionic and betaine co-emulsifiers, silicones, complexing agents, solvents, fragrances, vitamins, solvents, etc.

| Components | wt-% |
|---|---|
| Polymeric fatty acid | 0.50-5.00 |
| compound of the invention | |
| Cetyl Hydroxyethyl cellulose | 0.00-3.00 |
| Cetearyl alcohol | 0.00-3.00 |
| Citric acid | 0.00-2.00 |
| Glyceryl stearate and | 0.00-3.00 |
| PEG-100 stearate (ratio of glyceryl | |
| stearate:PEG-100 stearate from 0:1 to | |
| 1:0) | |
| Tetrasodium EDTA | 0.00-1.00 |
| Deionized water | q.s. 100% |

### Styling Gel for Hair

This formulation example is intended as a basic formulation. Formulations of this category customarily contain, but are not limited to, the following components: Fixative polymers, lacquers, acrylic acid derivatives, cellulose derivatives, vinyl derivatives, conditioning chemicals, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, etc.

| Components | wt-% |
|---|---|
| Polymeric fatty acid | 0.50-5.00 |
| compound of the invention | |
| Fixing agents | 0.10-10.00 |
| Hydroxyethyl cellulose | 0.00-2.00 |
| Citric acid | 0.00-2.00 |
| Fragrance | 0.00-5.00 |
| Deionized water | q.s. 100% |

### Styling Spray for Hair

This formulation example is intended as a basic formulation. Formulations of this category customarily contain, but are not limited to, the following components: Fixative polymers, lacquers, vinyl derivatives, fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethyl ether, compressed gases, etc.

| Components | wt-% |
|---|---|
| Polymeric fatty acid | 0.50-5.00 |
| compound of the invention | |
| Cyclomethicone | 0.00-80.00 |
| Fixing agents | 0.10-10.00 |
| Ethanol | 0.00-50.00 |
| Aerosol propellants | 0.00-50.00 |
| Preservatives | 0.00-0.50 |
| Fragrance | 0.00-5.00 |
| Deionized water | q.s. 100% |

### Pump Spray (Styling) for Hair

This formulation example is intended as a basic formulation. Formulations of this category customarily contain, but are not limited to, the following components: Vinyl derivatives, fixative polymers, lacquers, fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethyl ether, compressed gases, etc.

| Components | wt-% |
|---|---|
| Polymeric fatty acid | 0.50-5.00 |
| compound of the invention | |
| Cyclomethicone | 0.00-80.00 |
| Fixing agents | 0.10-10.00 |
| Ethanol | 0.00-50.00 |
| Preservatives | 0.00-0.50 |
| Fragrance | 0.00-5.00 |
| Deionized water | q.s. 100% |

The use of the polymeric fatty acid derivatives specified in the invention for applications in the hair care field produces favorable results with respect to strengthening, shine, fixing (hold), body, volume, moisture regulation, color retention, protection against environmental factors (LuV, salt water, etc.), manageability, combability, anti-frizz, anti-static properties, ability to dye, etc.

### Further Formulation Examples

In the following formulation examples, all values given represent the amount in “wt-% of the total composition” unless otherwise noted.

### Naturally Derived Crystal Clear, Betaine-Free Conditioning: Shampoo

Phase A

| | | |
|---|---|---|
| Aqua | | q.s. to 100 |
| Polyquta 400 KC (KCl Limited) | Anti-static cationic | 0.2 |
| (Polyquaternium-10) | polyquaternium | |
| | polymer | |

Phase B

| | | |
|---|---|---|
| Pureact WS Conc (Innospec) | mild anionic surfactant | 9.4 |
| (Sodium methyl cocoyl taurate) | | |
| Pureact Gluco L (Innospec) | Non ionic surfactant | 3.6 |
| (Lauryl glucoside) | foam boosting/cleanser | |
| Pureact MS-CG (Innospec) | Mild anionic surfactant | 3.6 |
| (Sodium methyl oleoyl taurate) | | |
| Pureact LSR (Innospec) | Mild anionic surfactant | 1.35 |
| (Sodium lauroyl sarcosinate) | | |

Phase C

| | | |
|---|---|---|
| Polymeric fatty acid | cationic | 1 |
| compound of the invention | conditioning | |
| | agent | |
| Surfac SB09 (Surfachem) | amphoteric surfactants | 9.4 |
| (Cocamidopropyl | | |
| hydroxysultaine) | | |

Phase D

| | | |
|---|---|---|
| Sodium benzoate | Preservatives | 0.5 |

Phase E

| | |
|---|---|
| Pomette (Azur Fragrances) | 0.5 |
| (Fragrance) | |

Phase F

| | | |
|---|---|---|
| Citric acid (50% w/w) | pH adjuster | q.s. to pH 4.2- |
| | | 4.7 |

Phase G

| | |
|---|---|
| Sodium chloride | qs to 4500- |
| | 8000 cps (max |
| | 0.8%) |

### Procedure

To a vortex of aqua Polyquta 400 KC was added and mixed until it is fully dispersed and clear. Phase A was heated to 40-45° C. Pureact WS Conc, Pureact Gluco L and Pureact MS-CG were homogenized by heating to 40-45° C. and mixing the products before adding to the main vessel. Sequentially, the ingredients in phase B were added and mix until uniform and clear. Slowly, Surfac SB09 & the polymeric fatty acid compound of the invention (phase C) were added to the main vessel and mixed until uniform. The vessel was cooled to below 40° C. and then the preservative was added, it was mixed until the mixture was clear and uniform. Fragrance was added and mixed until it is fully emulsified and clear. It was adjust pH to 4.2-4.7 with citric acid solution (50% w/w) as required. Small aliquots of sodium chloride, (0.2% w/w) were added as required until the desired viscosity was obtained.

### Fatal Attraction Hair Mist

Procedure
Phase A

| | | |
|---|---|---|
| Water | | 85.03 |
| Panthenol | Active | 0.5 |
| Dissolvine GL-38(AkzoNobel speciality chemicals) | Chelating | 0.15 |
| (Water, tetrasodium glutamate diacetate, sodium | agent | |
| hydroxide) | | |
| Gluconolactone SB (MakingCosmetics) | Preservative | 2 |
| (Gluconolactone, sodium benzoate, | | |
| calcium gluconate) | | |
| Propanediol | Humectant | 2 |

Phase B

| | | |
|---|---|---|
| Keracyn (Centerchem) (Propanediol, Water, | Antioxidant, | 1 |
| Glycerin, Cynara Scolymus (Artichoke) | acid | |
| Leaf Extract) | | |
| Sensfeel for Her (Centerchem) (Propanediol, | Fragrance | 5 |
| Jasminum Officinale Flower Extract, | | |
| Ceratonia Siliqua Fruit Extract, Phenethyl | | |
| Alcohol) | | |

Phase C

| | | |
|---|---|---|
| Fragrance | | 0.25 |
| Polymeric fatty acid | Cationic | 1 |
| compound of the invention | conditioning | |
| | polymer | |
| Caprylyl/capryl glycoside, polyglyceryl-4 | Nonionic | 2.5 |
| caprate, polyglyceryl-6 laurate, pentylene | surfactants | |
| glycol, sodium dilauramidoglutaminde lysin | | |

Phase D

| | |
|---|---|
| Sodium hydroxide, 20% | 0.57 |

### Procedure

Phase A was added to a vessel with gentle agitation. It was mixed until transparent. With agitation at RT, phase B was added into phase A. It was mixed until homogeneous. With continued agitation phase C was slowly added into Phase A/B with fitting mixer. When the mixture was uniform, the pH value was adjusted to 5.00-5.50 with phase D.

### Have A Peachy Day Jelly Shampoo

Phase A

| | | |
|---|---|---|
| Deionized water | | 39.95 |
| Endiquest GL-47S (Coast Southwest) | Chelating | 0.6 |
| (Tetrasodium glutamate diacetate) | agent | |
| Glycerin 99.7% USP Kosher (Coast | Humectant | 3 |
| Southwest, Inc.) (Glycerin) | | |
| Green Tea Concentrate (Coast Southwest, | Active | 2 |
| Tea Guys) (Water (and) camellia sinensis) | | |
| Synthalen W2000 (Coast Southwest, 3V | Anionic | 4 |
| Sigma-USA) (Acrylates/palmeth-25 acrylate | acrylic | |
| copolymer) | copolymer | |

Phase B

| | | |
|---|---|---|
| Endinol MILD B-SF65 (Coast Southwest) | Sulfate-free | 40 |
| (Sodium cocoyl isethionate (and) | surfactant | |
| cocamidopropyl hydroxysultaine (and) | package | |
| lauryl glucoside (and) cocamidopropylamine | | |
| (and) caprylyl/capryl glucoside) | | |
| NaOH 40% w/w solution | | q.s. |
| (Sodium hydroxide) | | |

Phase C

| | | |
|---|---|---|
| Yangu Oil (Coast Southwest, International | Active | 3 |
| Cosmetic Science Centre) (Calodendrum | | |
| capense nut oil) | | |
| Polymeric fatty acid | Cationic | 1 |
| compound of the invention | conditioning | |
| | polymer | |
| Cosmosil 660 Shea Oil (Coast Southwest, | Active | 1.5 |
| International Cosmetic Science Centre) | | |
| (Butyrospermum parkii (shea) oil) | | |
| Olivatis 19 (Coast Southwest, Medolla | Active | 3 |
| Limited) (Olive oil polyglyceryl-6 | | |
| esters (and) phospholipids) | | |

Phase D

| | | |
|---|---|---|
| Sharomix CPC30 (Coast Southwest, Sharon- | Preservative | 0.5 |
| Laboratories)(Phenylpropanol (and) | | |
| caprylyl glycol (and) chlorphenesin) | | |
| Fragrance | | 1.45 |

### Procedure

In the main vessel, the phase A ingredients in formula order were combined with shear mixing and heated to 140° F. to 149° F. (60-65° C.). Phase B was added to phase A in formula order with continuous mixing. The solution thickened once neutralized to desired pH. In a separate vessel, phase C ingredients were combined and heated to 140° F. to 149° F. (60-65° C.). Once uniform, phase C was added to phase AB under propeller mixing. Phase D was combined in a separate vessel, then added to the main vessel under continuous mixing. The mixture was transfer to the final container.

### PEG-Free Shampoo

Phase A

| | | |
|---|---|---|
| Standapol ES-2 (BASF) (Sodium laureth | Anionic | 25.2 |
| sulfate) | Surfactant | |
| Plantaren 2000 N UP (BASF) (Decyl | Nonionic | 15.1 |
| glucoside) | surfactant | |
| Lexaine C (Inolex) (Cocamidopropyl betaine) | Amphoteric | 10.2 |
| | surfactant | |
| Sulfochem AEG Surfactant Blend (Lubrizol) | Surfactant blend | 4 |
| (Ammonium lauryl sulfate (and) ALES (and) | | |
| CAPB (and) cocamide DEA (and) lauramide | | |
| DEA) | | |
| Glycerin, USP (Dow Chemical) (Glycerin) | Humectant | 0.5 |

Phase B

| | | |
|---|---|---|
| Polymeric fatty acid | Cationic | 1 |
| compound of the invention | conditioning | |
| | polymer | |
| Floraesters K-20W Jojoba (Floratech) | Oil | 5.3 |
| (Hydrolyzed jojoba esters (and) Floratech | | |
| water (Aqua)) | | |

Phase C

| | | |
|---|---|---|
| Deionized water | | q.s. |
| | | 100 |
| Quatrisoft Polymer LM-200 (Dow Chemical) | cationic | 0.1 |
| (Polyquaternium-24) | polyquaternuim | |
| | polymer | |
| Tauranol I-78 (Innospec Performance | Isethionate | 4.7 |
| Chemicals) (Sodium cocoyl isethionate) | surfactant | |

Phase D

| | |
|---|---|
| Preservative | q.s. |
| Fragrance | q.s. |
| Color | q.s. |

### Procedure

The ingredients of phase A were mixed with moderate propeller agitation while heating to 70° C. until uniform. Phase B was added to phase A and mixed until uniform. Deionized water of phase C was heated to 65-70° C. and the the Quatrisoft Polymer LM-200 was dissolved. Slowly Tauranol 1-78 was added once the Quatrisoft Polymer LM-200 had completely dissolved. Phase C was slowly added to phase AB. It was mix until uniform and cooled to 50° C. Phase D was added in the order listed to Phase ABC with moderate propeller agitation. The mixture was cooled to room temperature.

### Repairing Shampoo Bar

Phase A

| | | |
|---|---|---|
| Sodium Cocoyl Isethionate, Stearic Acid | Anionic | 38 |
| | surfactant with | |
| | fatty acid | |
| Water | | 8.7 |
| Citric Acid, 50% | | 0.5 |
| AMA-PROT (Centerchem) (Water, Glycerin, Amaranthus Caudatus | Extract | 3 |
| Seed Extract, Zea Mays (Corn) Starch.) | | |
| KERACYN (Centerchem) (Propanediol, Water, Glycerin, Cynara | Extract | 3 |
| Scolymus (Artichoke) Leaf Extract.) | | |
| BAICAPIL(Centerchem) (Propanediol, Water, Arginine, Lactic Acid, | Active | 2 |
| Glycine Soja (Soybean) Germ Extract, Triticum Vulgare (Wheat) | | |
| Germ Extract, Scutellaria Baicalensis Root Extract.) | | |

Phase B

| | | |
|---|---|---|
| Sucrose Palmitate | Nonionic | 1 |
| | surfactant | |
| Sodium Methyl Cocoyl Taurate | Mild anionic | 27 |
| | solid surfactant | |
| Erythritol | | 6 |

Phase C

| | | |
|---|---|---|
| Polymeric fatty acid | Cationic | 1 |
| compound of the invention | conditioning | |
| | polymer | |
| VITAOILS PLUS (Centerchem) (Helianthus Annuus (Sunflower) | Oils | 8 |
| Seed Oil, Cocos Nucifera (Coconut) Oil, Linum Usitatissimum | | |
| (Linseed) Seed Oil, Persea Gratissima (Avocado) Oil, Argania | | |
| Spinosa Kernel Oil, Macadamia Ternifolia Seed Oil.) | | |
| Sucrose Tetrastearate Triacetate | Fatty acid | 1.5 |
| | modifed sugar | |

Phase D

| | |
|---|---|
| Fragrance | 0.3 |

### Procedure

In the main vessel, the components of Phase A were added with gentle mixing, and it was heated to 70-75° C. Phase B was added into Phase A with continued mixing and maintaining a temperature of 70-75° C. Phase C was added into Phase AB with continued mixing and maintaining a temperature of 70-75° C. When the batch is uniform, it was cooledPhase D was added to the batch. When the batch was cooled to 45° C., the pre-heated sticks were filled. The sticks were placed in a freezer for 12-24 hrs. before the first use.

### Frozen Yogurt Hair Mask

Phase A

| | | |
|---|---|---|
| Deionized water (Water) | | 57.64 |
| Phytic Acid Extreme (Phytic acid, water) | Active | 0.5 |
| Liponic Bio EG-1 (Glycereth-26) | Humectant/ | 5 |
| | emulsifier | |

Phase B

| | | |
|---|---|---|
| Carbopol Ultrez 21 (Acrylates/C10-30 | polyacrylic | 0.35 |
| alkyl acrylate crosspolymer) | acid | |
| | derivative | |

Phase C

| | |
|---|---|
| Sodium hydroxide 10% solution (Water, | 1.91 |
| sodium hydroxide) | |

Phase D

| | | |
|---|---|---|
| Lipomulse Luxe MB (Vantage Personal | Non ionic | 3 |
| Care) (Cetearyl alcohol, glyceryl | Surfactant/ | |
| stearate, PEG-40 stearate, ceteareth-20) | Emulsifier | |
| Lipovol C-76 (Cocos nucifera (coconut) oil) | conditioning | 6 |
| | Oil | |
| Polymeric fatty acid | Cationic | 1 |
| compound of the invention | condioning | |
| | polymer | |
| Avocado oil organic (Persea gratissima | Conditioning | 6 |
| (avocado) oil) | oil | |
| Iso Jojoba 35 (Simmondsia chinensis | Conditioning | 3 |
| (jojoba) butter) | oil | |

Phase E

| | |
|---|---|
| Preservative | 0.6 |

Phase F

| | | |
|---|---|---|
| Coconut Avocado Hair Milk (Water, cocos nucifera | Active | 15 |
| (coconut) oil, persea gratissima (avocado) oil, | | |
| propanediol, glyceryl stearate, phospholipids, | | |
| cocos nucifera (coconut) water, cocos nucifera | | |
| (coconut) fruit juice, polyglyceryl-10 oleate, | | |
| polyglyceryl-10 dioleate, cetearyl alcohol, sodium | | |
| stearoyl lactylate, glycerin) | | |

### Procedure

In the main beaker, Phase A was weighed and heated to 75° C. Phase B was sprinkled on the aqueous phase and it was waited until carbopol was fully hydrated. It was homogenized, Phase C was added to neutralize, and it was homogenized again. In an annex container, Phase D was weighed and heated to 75° C. D was added slowly into the aqueous phase under High stirring. Then, the emulsion was cooled down with moderate stirring. At 35° C., Phase E and Phase F were added and homogenized.

### Crystal Clear Healthy Hair Shampoo

Phase A

| | | |
|---|---|---|
| Deionized Water | | 59 |
| Hostapon SCI 85P (Clariant) (Sodium | Mild anionic | 3.5 |
| cocoyl isethionate) | Surfactant | |
| Glucotain Plus (Clariant) (Capryloyl/ | Mild nonionic | 10 |
| Caproyl Methyl Glucamide (and) | Surfactant | |
| Lauroyl/Myristoyl Methyl Glucamide) | | |
| Genopal LT (Clariant) (PEG 150 PG-2) | PEG based | 1.5 |
| | Thickner | |
| Amphosol CS-50 (Stepan)(Cocamidopropyl | Amphoteric | 9 |
| Hydroxysultaine) | Surfactant/foam | |
| | booster | |

Phase B

| | | |
|---|---|---|
| Glucquat 125 (Lubrizol) (Lauryl Methyl | Humenctant | 1.5 |
| Gluceth-10 HydroxypropylDimonium Chloride) | | |
| Celquat 240C (Polyquaternium-10 | Cationic | 0.15 |
| (Akzo Nobel) | polyquaternium | |
| | conditioner | |
| Deionized water | | 10 |

Phase C

| | | |
|---|---|---|
| Polymeric fatty acid | cationic | 1 |
| compound of the invention | conditioning | |
| | agent | |
| Nipaguard SCP (Clariant) (Phenoxyethanol | Preservative | 1 |
| (and) Sorbitan Caprylate) | | |

Phase D

| | |
|---|---|
| Sodium Hydroxide/Citric Acid | q.s. |
| Deionized Water | q.s. |

### Procedure

In an appropriate vessel the water was added. While the water was heated to 80-85° C., the Hostapon SCI, Glucotain Plus and Amphosol CS-50 were added. With the temperature at 80-85° C., it was mixed until uniform and removed from the heat. In a separate beaker, Celquat 240C, Glucquat 125 and Deionized water were mixed until uniform. Once homogenous, Celquat/Gluquat Blend were added to the Main Batch (Phase A). Phase C ingredients were added one by one and mixed well. The solution pH was adjusted with 20% citric acid or 20% NaOH to pH 6.0 to 6.5. The batch was filled to 100% with deionized water.

### Hair Repairing Serum

Phase A

| | | |
|---|---|---|
| Deionized water | | 61 |
| Tetrasodium glutamate diacetate, | Stabilizer | 0.2 |
| sodium hydroxide, water | | |
| Hydroxyethyl cellulose | Thickner | 2 |
| Potassium sorbate | Stabilizer/preservative | 0.15 |
| Sodium benzoate | Preservatives | 0.15 |

Phase B

| | | |
|---|---|---|
| Water | | 19.92 |
| Polyquaternium-16 | Cationic | 0.5 |
| | polyquarternium | |
| | polymer | |

Phase C

| | | |
|---|---|---|
| Phenethyl alcohol | Masking agent | 0.8 |
| Polymeric fatty acid | Cationic condioning | 1 |
| compound of the invention | agent | |
| PPG-26 butheth-26, PEG-40 | non-ionic surfactants | 5.4 |
| hydrogenated castor oil | | |
| Propanediol | humectant | 1.25 |

Phase D

| | | |
|---|---|---|
| Lactic acid, 50% | pH adjuster | 0.13 |

Phase E

| | | |
|---|---|---|
| Amber Extract MS (Provital | Active | 2.5 |
| S.A./Centerchem Inc.) | | |
| Keratrix (Provital | Active | 5 |
| S.A./Centerchem Inc.) | | |

### Procedure:

In separate vessel, the components of phase A were added separately with mixing while heating to a temperature of 50° C. It was mixed until uniform and homogeneous. The mixture was cooled to a temperature<35° C. Each in separate vessel, the components of phase B and phase C were added separately with mixing and mixed until uniform. When the main vessel had cooled to 35° C., phases B and C were added to phase A. It was mixed until uniform. Phase D was added to the main vessel to adjust pH to 4.80-5.40. Phase E was added to the main vessel with gentle agitation, and it was mixed until uniform.

Hair Repairing Serum with Keratrix

Phase A

| | | |
|---|---|---|
| Deionized water | | 66 |
| Dissolvine GL-38(AkzoNobel speciality | Chelating | 0.2 |
| chemicals) (Water, tetrasodium | agent | |
| glutamate diacetate, sodium | | |
| hydroxide) | | |
| Hydroxyethyl cellulose | Suger based | 2 |
| | thickner | |
| Potassium sorbate | | 0.15 |
| Sodium benzoate | | 0.15 |

Phase B

| | | |
|---|---|---|
| Water | | 19.92 |
| Polyquaternium-16 | Cationic Poly- | 0.5 |
| | quarternium | |
| | polymer | |

Phase C

| | | |
|---|---|---|
| Phenethyl Alcohol | | 0.8 |
| Polymeric fatty acid | Cationic | 1 |
| compound of the invention | conditioning | |
| | polymer | |
| Solubilisant LRI (Sensient Cosmetic & | non-ionic | 0.4 |
| Fragrances) (PPG-26 Butheth-26, | surfactants | |
| PEG-40 hydrogenated castor oil) | mixture | |
| Propanediol | | 1.25 |

Phase D

| | |
|---|---|
| Lactic acid, 50% | 0.13 |

Phase E

| | |
|---|---|
| Amber Extract MS (Provital/Centerchem) | 2.5 |
| Keratrix (Provital/Centerchem) | 5 |

### Procedure

In separate vessel, the components of phase A were added separately with mixing while heating to a temperature of 50° C. It was mixed until uniform and homogeneousand the mixture was cooled to a temperature<3500. Each in a separate vessel, the components of phase B and phase C were added separately with mixing, and mixed until uniform. When the main vessel had cooled to 3500, phases B and C were added to phase A, and it was mixed until uniform. Phase D was added to the main vessel to adjust pH to 4.80-5.40. Phase E was added to the main vessel with gentle agitation, and it was mixed until uniform.

### Put More Life In Your Hair Clay Mask

Phase A

| | | |
|---|---|---|
| Deionized water (Deionized water) | | 70.9 |
| Dissolvine NA2-S (Coast Southwest, Akzo | | 0.5 |
| Nobel Functional Chemicals) (Disodium EDTA) | | |
| Glycerin 99.7% USP Kosher (Coast Southwest) | | 2 |
| Conditioner P10 (Coast Southwest, 3V Sigma | cationic | 0.8 |
| USA)(Polyquaternium-10) | polyquaternium | |
| | polymer | |

Phase B

| | | |
|---|---|---|
| Polymeric fatty acid | Cationic | 1 |
| compound of the invention | conditioning | |
| | polymer | |
| Olivatis 19 (Coast Southwest, Medolla Limited) | polyglyceryl | 3 |
| (Olive oil polyglyceryl-6 esters (and) | ester with | |
| phospholipids) | phospholipids | |
| Olivatis 18 (Coast Southwest, Medolla Limited) | polyglyceryl | 4 |
| (Olive oil polyglyceryl-6 esters (and) sodium | ester with | |
| stearyl Lactylate (and) cetearyl alcohol) | fatty alcohols | |
| Cosmodan 20 (Coast Southwest, International | | 4 |
| Cosmetic Science Centre) (Elaeis guineensis | | |
| (palm) oil (and) brassica campestris (rapeseed) | | |
| seed oil) | | |
| Tamanu Butter (Coast Southwest, Inc., | Butter | 2 |
| International Cosmetic Science Centre) | | |
| (Calophyllum inophyllum seed oil (and) | | |
| butyrospermum parkii nut extract) | | |
| Kpangnan Butter (Coast Southwest, International | | 2 |
| Cosmetic Science Centre) (Pentadesma | | |
| butyraceae seed butter) | | |
| Coconut Olein (Coast Southwest, International | | 1 |
| Cosmetics Science Centre) (Cocos nucifera | | |
| (coconut) oil) | | |
| Cosmosil B (Coast Southwest, Inc., International | Oil | 0.5 |
| Cosmetic Science Centre) (Brassica campestris | | |
| seed oil (and) oryza sativa bran oil) | | |
| Endimate 33V (Coast Southwest) (Caprylic/capric | vegetable- | 1 |
| triglyceride) | origin, | |
| | medium chain | |
| | triglyceride | |
| Glossamer L6600 (Coast Southwest) (Brassica | | 2 |
| campestris/aleurites fordi oil copolymer) | | |

Phase C

| | | |
|---|---|---|
| Pelavie Yellow Clay (Coast Southwest, Inc., The | Clay | 4 |
| Innovation Company) (Bentonite) | | |

Phase D

| | |
|---|---|
| Sharomix 704 (Coast Southwest., Sharon | 0.8 |
| Laboratories) (Benzoic Acid (and) dehydroacetic | |
| acid (and) phenoxyethanol) | |

### Procedure:

Phase A ingredients were combined in formula order into main vessel with propeller mixing and heated to 60-70° C. In a separate vessel, the phase B ingredients in formula order were combined under propeller mixing and it was heated to 60-70° C. Once both phase A and phase B were fully uniform, phase B was added to phase A with continuous mixing. Once fully dispersed, heat was discontinued. Once the temperature is at 35-40° C., phase C was added to phase AB with continuous mixing. Phase D was added to Phase ABC, mixing was discontinued and it was switched to a homogenizer. The mixture was homogenized for 10-30 seconds. Once complete, the mixture was transferred to a holding vessel.

### Detox Hair Sleeping Pack

Phase A

| | | |
|---|---|---|
| Water | | 87.03 |
| Panthenol | Multifuctional benefits | 2 |
| Ethylhexylglycerin | Preservative | 0.5 |
| Citric acid, 50% | pH adjuster | 0.6 |
| Sodium methylparaben | Preservative | 0.25 |
| Lexgard Natural MB (INOLEX | Emollient | 1 |
| Incorporated)Glyceryl | | |
| caprylate, glyceryl undecylenate | | |

Phase B

| | | |
|---|---|---|
| Polymeric fatty acid | cationic conditioning | 1 |
| compound of the invention | agent | |
| Acrylkates/C10-30 alkyl acrylate | polyacrylic acid | 1 |
| crosspolymer | derivative | |

Phase C

| | | |
|---|---|---|
| Ama-leaf (Provital S.A./Centerchem) | Active | 1 |
| Kercyn (Provital S.A./Centerchem) | Active | 2 |
| Pronalen bio-protect znsn | Active | 2 |
| (Provital S.A./Centerchem) | | |
| Sodium hydroxide, 20% | | 1.62 |

### Procedure

In main vessel, the components of phase A were added separately with gentle mixing until the mixture was uniform. Phase B was dispersed into the vessel with high shear mixing. When the batch was uniform, the components of phase C were added individually, wherein it was mixed until uniform before the next addition. The pH value was adjusted to 5.40-6.00 with sodium hydroxide, and it was mixed until the gel was uniform.

### Clean Beauty Light & Clean Conditioner

Phase A

| | |
|---|---|
| Water, deionized (water) | 89.8 |

Phase B

| | | |
|---|---|---|
| Emulsense HC (Inolex) (Brassicyl | cationic anti- | 1.5 |
| isoleucinate esylate, brassica alcohol) | static agent | |
| | and emulsifier | |
| Argan Oil (DSM Nutritional Products) | Conditioning oil | 0.5 |
| (Argania spinosa kernel oil) | | |
| Cetyl alcohol | co-surfactant | 0.7 |
| Neossance Hemisqualane CN | Emollient | 1 |
| (Aprinnova/Centerchem) (C13-16 isoparaffin) | | |

Phase C

| | | |
|---|---|---|
| Polymeric fatty acid | cationic | 1 |
| compound of the invention | conditioning | |
| | agent | |
| Ama-prot (Provital S.A./Centerchem) (Water, | Active | 1.5 |
| glycerin, amaranthus caudatus seed extract, | | |
| zea mays starch) | | |
| Baicapil (Provital S.A./Centerchem) | Active | 2 |
| (Propanediol, water, arginine, lactic acid, | | |
| glycine soja(soybean) germ extract, triticum | | |
| vulgare (wheat) germ extract, Scutellaria | | |
| baicalensis root extract) | | |
| Leucidal SF Max (Active Micro Technologies) | Active | 2 |
| (Lactobacillus ferment) | | |

### Procedure

Phase A was heated to 75° C. under agitation. In a separate vessel, the ingredients of phase B were heated to 75° C. under agitation. Phase B was added to phase A and mixing for 10 minutes was continued. The heat was removed and stirring was continued until the product reached 40° C. The ingredients of phase C were combined and mixed well under medium agitation. Phase C was added to phase A/B at 40° C. and stirring was continued until the product reached room temperature.

### Gently Bubbles It' Mild Shampoo

Phase A

| Water (Aqua) | q.s. to |
|---|---|
| | 100 |

Phase B

| | | |
|---|---|---|
| Polyquta 400 KC (KCl Limited) (Polyquaternium-10) | cationic | 0.2 |
| | polyquaternium | |
| | polymer | |

Phase C

| | | |
|---|---|---|
| Iselux Ultra Mild (Innospec) (Aqua, sodium lauroyl methyl | Mild anionic, | 32 |
| isethionate, cocamidopropyl betaine, sodium methyl oleoyl | amphoteric & non | |
| taurate, lauryl glucoside, coco-glucoside) | ionic surfactants | |

Phase D

| | | |
|---|---|---|
| Polymeric fatty acid | cationic conditioning | 2 |
| compound of the invention | agent | |
| Emulsil S-393 (Innospec) (PEG-12 dimethicone) | hydrophilic silicones | 0.75 |

Phase E

| | |
|---|---|
| Odersynthesis fragrance (Intarome) (Fragrance) | 0.25 |

Phase F

| | | |
|---|---|---|
| Euxyl K100 (Schülke) (Methylchloroisothiazolinone, | preservative | 0.05 |
| methylisothiazolinone, benzyl alcohol) | | |

Phase G

| | | |
|---|---|---|
| Citric acid (50% solution) (Citric acid, water) | pH adjuster | q.s. to |
| | | pH 5.5- |
| | | 6.0 |

### Procedure

Water was charged into a mixing vessel and Polyquaternium-10 (B) was sprinkled into the water, and it was mixed until clear. With moderate mixing Iselux Ultra Mild (C) was poured into the main vessel. Phase (D) was mixed to the batch. It was mix until clear, then the desired fragrance and preservative was added and the pH was adjusted using citric acid (50% w/w solution) (G).

### Coconut Dream Conditioner

Phase A

| Water | | to |
|---|---|---|
| | | 100.00 |
| Dehyquart A-CA (BASF) | cationic | 0.5 |
| (Cetrimonium chloride) | Surfactant | |
| Sodium EDTA | | 0.15 |

Phase B

| | | |
|---|---|---|
| Crodacol C90 (Croda) (Cetyl | Emulsion | 1.5 |
| alcohol) | stabiliser | |
| Crodacol S95 (Croda) (Stearyl | Emulsion | 2.5 |
| alcohol) | stabiliser | |
| Polymeric fatty acid | cationic | 1 |
| compound of the invention | conditioning | |
| | agent | |
| Coconut Oil | conditioning oil | 0.3 |
| Arquat 2HT-75 PG (Akzo Nobel) | cationic quaternary | 0.8 |
| (Quaternium-18, propylenglycol) | conditioning agent | |
| Eumulgin B2 (BASF) | non-ionic | 0.5 |
| (Ceteareth-20) | emulsifier | |

Phase C

| | | |
|---|---|---|
| Tocopherolacetate | active | 0.2 |
| BeauSil AMO 8950 EM (CHT) | Amodimethicone | 3.5 |
| (Amodimethicone, cetrimonium | | |
| chloride, trideceth-12) | | |

Phase D

| | | |
|---|---|---|
| Euxyl K320 (schülke) | Preservative | 0.5 |
| (Preservative agent) | | |
| Fragrance | | q.s. |

### Procedure

The ingredients of Phase A were mixed and heat to 80° C. The ingredients of phase B were blended at 80° C. Phase B was added to phase A. It is cooled down to 40° C. and phase C is added. It is cooled further, phase D is added, and the mixture is adjusted to pH 4.3-4.7.

### Glycolic Acid Shampoo

Phase A

| | | |
|---|---|---|
| Deionized water | | 51.3 |
| Glycerin (Coast Southwest) | Humenctant | 4 |
| Endiquest GLDA (Coast Southwest) | Stabilizer | 0.2 |
| (Tetrasodium glutamate diacetate) | | |
| Synthalen W2000 (Coast Southwest, | anionic acrylic | 5 |
| 3V-Sigma USA) (Acrylates/palmeth-25 | copolymer | |
| acrylate copolymer) | | |

Phase B

| | | |
|---|---|---|
| Deionized water | | 5 |
| GlyAcid 70 (Coast Southwest, | active | 4 |
| CrossChem) (Glycolic acid) | | |
| NaOH (30% aq.) (Sodium hydroxide) | | q.s. |

Phase C

| | | |
|---|---|---|
| Endinol Mild SF-65 (Coast Southwest) | Mild anionic, | 20 |
| (Sodium cocoyl isethionate, | nonanionic & | |
| cocamidopropyl hydroxysultaine, | amphoteric | |
| lauryl glucoside, cocamidopropylamine | Surfactant blend | |
| oxide, caprylyl/capryl glucoside) | | |
| Polymeric fatty acid | cationic | 1 |
| compound of the invention | conditioning | |
| | agent | |
| GlucoTain Clear (Coast Southwest, Clariant) | Nonionic | 5 |
| (Capryloyl/caproyl methyl glucamide) | Surfactants | |
| Enditeric COAB (Coast Southwest) | Amphoteric | 5 |
| (Cocamidopropyl betaine) | surfactant | |

Phase D

| | |
|---|---|
| NaOH (30% aq.) (Sodium hydroxide) | q.s. |

### Procedure

In the main vessel, phase A was added and mixed until uniform. Phase B is added to phase A. In a side vessel, phase C is combined, then it was add slowly to phase AB, wherein the pH is required to be >4. The preservative was added to phase ABC. While the batch was initially discontinuous, mixing was continued. -Slowly add the surfactant to phase ABCD. The batch will become uniform and increase in viscosity. Finally fragrance was added.

### 2-Phase Super Hydration Hair Treatment

Phase A

| | |
|---|---|
| Water (aqua) | to 100.00 |

Phase B

| | | |
|---|---|---|
| Jaguar C-162 (Solvay) (hydroxypropyl | | 0.9 |
| guar, hydroxypropyltrimonium chloride) | | |
| Glycerin (Merck) | humectant | 0.9 |

Phase C

| | | |
|---|---|---|
| Lactic Acid 80 (Lactic acid) | pH adjuster | 0.15 |
| BeauSil AMO 918 (CHT) (Gluconamido | Amodimethicone | 1.1 |
| amodimethicone, trideceth-7, trideceth-8) | | |
| Panthenol | multifictional | 0.2 |
| | active | |
| Sodium benzoate | Preservative | 0.1 |

Phase D

| | | |
|---|---|---|
| Polymeric fatty acid | cationic | 1 |
| compound of the invention | conditioning | |
| | agent | |
| BeauSil Fluid 8301 (CHT) (C13-15 | emolient | 7 |
| alkane, Isododecane, caprylyl methicone) | | |
| BeauSil PEG 010 (CHT) (PEG/PPG-15/5 | ambiphilic | 1 |
| dimethicone) | surfactant | |
| BeauSil Gum 8501 (CHT) (C13-15 | conditioning | 5 |
| alkane, isododecane, caprylyl methicone, | agent | |
| dimethiconol) | | |

Phase E

| | |
|---|---|
| Dye and fragrance | q.s. |

### Procedure

The ingredients of phase B were blended and added to phase A while mixing. Then the ingredients of phase C were added to phase AB. The ingredients of phase D were blended and added with high-shear to phase ABC. Then phase E was added.

### Cleanse & Nourish Oil Shampoo

| Phase A | | |
|---|---|---|
| Water deionized (Aqua) | | to 100 |
| Crodateric CAS 50 (Croda) (Cocamidopropyl | Amphoteric | 8 |
| hydroxysultaine (and) water (aqua)) | surfactant | |
| Crodateric CAB 30 (Croda) (Cocamidopropyl | Amphoteric | 15 |
| betaine (and) water (aqua)) | surfactant | |
| Jeelate ES-3 (Jeen) Sodium laureth | Anionic | 35 |
| sulfate 30%) | surfactant | |
| Phase B | | |
| Versathix (Croda) (PEG-150 pentaerythrityl | | 1.5 |
| tetrastearate (and) PPG-2 hydroxyethyl | | |
| cocamide (and) water (aqua)) | | |
| Crovol A70 (Croda) (PEG-60 almond | Nonionic | 2 |
| glycerides) | emollient | |
| Cromollient SCE (Croda) (Di-PPG-2 | | 0.5 |
| myreth-10 adipate) | | |
| Polymeric fatty acid | cationic | 1 |
| compound of the invention | conditioning | |
| | agent | |
| Ariasilk EFA (Croda) (Linoleamidopropyl | cationic | 1.5 |
| PG- dimonium chloride phosphate (and) | surfactant | |
| propylene glycol (and) water (aqua)) | | |
| Cropure Almond (Croda) (Prunus amygdalus | | 0.25 |
| dulcis (sweet almond) oil) | | |
| Procetyl AWS (Croda) (PPG-5-ceteth-20) | | 3 |
| Phase C | | |
| Phytessence French Oak (Crodarom) (Water | | 0.5 |
| (aqua) (and) glycerin (and) quercus | | |
| petraea fruit extract) | | |
| Neolone 950 (Dow) | | 0.1 |
| (Methylchloroisothiazolinone) | | |
| Citric acid (25% solution) | | 0.07 |
| Sodium chloride | | 0.5 |

### Procedure

Phase A was heated to 75-80° C. Phase B was premixed and added to part A with medium speed mixing. It was cooled to 40° C. and Phase C was added. The pH was checked and adjusted if necessary using citric acid solution.

### Hair Wax Formulation

Phase A

| Water (aqua) (deionized) | to 100 |
|---|---|
| Propylene glycol | 10 |
| Sorbitol | 7 |

Phase B

| | | |
|---|---|---|
| Mineral oil (Paraffinum liquidum) | | 12 |
| Sensolene Care DD (Hallstar | fatty acid/esters | 3 |
| Italia) (Lauryl olivate) | | |
| Steareth-20 | Nonionic surfactant | 20 |
| Steareth-2 | Nonionic surfactant | 3 |
| Polymeric fatty acid | cationic | 1 |
| compound of the invention | conditioning | |
| | agent | |
| SALCARE SC 96 (BASF) | cationic | 2.5 |
| (Polyquaternium-37, propylene | polyquaternium | |
| glycol dicaprylate/dicaprate, | polymer | |
| PPG-1 trideceth-6) | | |
| BHT | | 0.1 |

Phase C

| | |
|---|---|
| Silica, titanium dioxide, tin oxide | 0.5 |

Phase D

| | |
|---|---|
| Preservative | a.n. |
| Fragrance (Parfum) | a.n. |

### Procedure

Phase A was prepared and heated to 75-80° C. Phase B was prepared and heated to 70-75° C. Phase B was added to phase A and homogenized for a few minutes using a suitable dispersion unit (e.g. Silverson, Ultra Turrax, etc.). It was cooled to 40° C., and the phases. C and D were added and mixed for a few minutes. The mixture was cooled to room temperature.

### On-the-Go Hair Sherbet

| Phase A | | |
|---|---|---|
| Water | | 81.25 |
| Trisodium ethylenediamide disuccinate | | 0.15 |
| Glyceryl caprylate, glyceryl undecylenate | Non ionic | 0.5 |
| | surfactant | |
| Panthenol | | 0.3 |
| Pentylene glycol | | 3 |
| Phase B | | |
| Dehydroxanthan gum | | 0.6 |
| Sodium polyacrylate starch | polyacrylate | 0.5 |
| | derivative | |
| Phase C | | |
| Polymeric fatty acid | cationic | 1 |
| compound of the invention | conditioning | |
| | agent | |
| SME 253 PF (Momentive performance | Amodimethicone | 4 |
| materials) Amodimethicone, C11-15 pareth-7, | | |
| laureth-9, glycerin, trideceth-12 | | |
| Lauryl methyl glyceth-10 | cationic polymer | 0.5 |
| hydroxypropyldimonium | | |
| chloride | | |
| Isopentyldiol | | 4 |
| Phase D | | |
| Keracyn (Provital S.A./Centerchem Inc.) | | 2 |
| Keranutri (Provital S.A./Centerchem Inc.) | | 2 |
| Lactic Acid, 50% | | 0.07 |
| Bismuth Oxychloride, mica, chromium oxide | | 0.03 |
| green (CI 77299) | | |
| Fragrance | | 0.1 |

### Procedure

Phase A was added to a vessel with gentle agitation while heating to 45-50° C. It was mixed until uniform. With agitation, phase B was added into phase A. It was mixed until homogeneous. With continued agitation, phase C was added into phase A/B. When uniform, phase D ingredients were added individually to phase A/B/C with gentle agitation Between each addition it was mixed.

### Dreamy Curls 24-Hr Weightless Foam

| | | |
|---|---|---|
| Phase A | | |
| Deionized water (Aqua) | | 92.66 |
| Styleze ES-1 polymer (Ashland) (Guar | | 1 |
| hydroxypropyltrimonium chloride) | | |
| Citric Acid (20% aq. Solution) (Local) (Citric acid) | | 0.24 |
| Benecel E4M HPMC (Ashland) (Hydroxypropyl | | 0.1 |
| methylcellulose) | | |
| Amphosol CA (Stepan) (Cocamidopropyl betaine) | Amphoteric | 3 |
| | surfactant | |
| Polymeric fatty acid | cationic | 1 |
| compound of the invention | conditioning | |
| | agent | |
| Glycerin USP (Jeen International) (Glycerin) | | 1 |
| Optiphen BSB-W preservative (Benzyl alcohol, | | 1 |
| aqua (water), sodium benzoate, potassium sorbate) | | |

### Procedure:

Water was added to the main container and mixed with propeller agitation. Styleze ES-1 was added into the vortex to disperse. Citric acid was added and mixed for approximately 10-15 min Benecel E4M was added and mixed until no particles were seen. Theolymeric fatty acid compound of the invention, Amphosol CA, glycerin and Optiphen BSB-W were added one by one and mixed until uniform.

Sea Salt 2-in-1 Scalp Treatment Shampoo

| Phase A | | |
|---|---|---|
| Water | | q.s. to 100 |
| Glycerin | | 7 |
| Polyquta 400 KC (KCl) | Cationic | 0.1 |
| (Polyquaternium-10) | polyquaternium | |
| | polymer | |
| Phase B | | |
| Empigen BB (Innospec) | amphoteric | 3 |
| (Lauryl betaine) | surfactant | |
| Pureact Gluco C (Innospec) | Nonionic | 3 |
| (Coco-glucoside) | surfactant | |
| Pureact WS Conc (Innospec) (Sodium | Mild anionic | 17 |
| methyl cocoyl taurate) | surfactant | |
| Phase C | | |
| Empilan EGDS/A (Innospec) (Glycol | Nonionic | 2.5 |
| distearate) | surfactant | |
| Iselux (Innospec) (Sodium lauroyl methyl | Mild anionic | 8 |
| isethionate) | isethionate | |
| Phase D | | |
| Citric acid (50% w/w solution) (Water, citric | | q.s. to pH |
| acid) | | 5.5-6.0 |
| Phase E | | |
| Polymeric fatty acid | Cationic | 1 |
| compound of the invention | conditioning | |
| | Polymer | |
| Macadamia Nut Oil (Macadamia integrifolia | | 7 |
| (macadamia) seed oil) | | |
| Jojoba Seed Oil (Vantage) (Simmondsia | | 7 |
| chinesis (jojoba) seed oil) | | |
| Phase F | | |
| Cetearyl Alcohol (Naturally Thinking) | | 3 |
| Phase G | | |
| Coarse sea salt crystals (Sodium chloride) | | 40 |
| Orchid 107745 (Sozio) (Fragrance) | | 0.5 |

### Procedure

Polyquaternium-10 was slowly added to water. Next, glycerin was added in the main vessel and mixed until fully dispersed and clear. Sequentially, Phase (B) ingredients were added and it was heated to 65-70° C. and mixed until homogenous. Phase (C) ingredients were added, the temperature was maintained at 65-70° C. and it was mixed until homogenous. The pH was adjusted with citric acid (50% w/w solution) to 5.5-6.0. Phase (E) ingredients were added and mixed until homogenous. Phase (F) was added while keeping the temperature at ˜65° C. and it was mixed until structure was obtained. Heating was stopped and cooled to 30° C., Phase (G) ingredients were added with mixing.

### Anti-Humidity Sparkling Hair Serum

| | | |
|---|---|---|
| Phase A | | |
| Avocado Oil (Provital/Centerchem) | | 3 |
| AMA-Oil (Provital/Centerchem) | | 0.5 |
| Cyclopentasiloxane | cyclomethicone | 49 |
| Polymeric fatty acid | cationic | 1 |
| compound of the invention | conditioning | |
| | agent | |
| Vegelight 1214LC (Grant Industries, Inc.) | | 30.05 |
| Coconut Alkanes, Coco-Caprylate/Caprate | | |
| Tocopherol | | 0.2 |
| Dicaprylyl Ether | | 16 |
| Phase B | | |
| Fragrance | | 0.15 |
| Phase C | | |
| KTZ SM INTERVAL BLUE (Kobo | | 0.1 |
| Products, Inc) Synthetic Fluorphlogopite, | | |
| Titanium Dioxide | | |

### Procedure:

In the main vessel, the components of Phase A were added separately with gentle mixing until the mixture was uniform and transparent. The remaining Phases were added individually, wherein it was mixed until uniform before the next addition.

D5 Free Primer with Argan Oil

| | | |
|---|---|---|
| Phase A | | |
| BeauSil Fluid 8301 (CHT) (C13-15 | Dimethicone | to |
| alkane, isododecane and caprylyl | | 100.00 |
| methicone) | | |
| Polymeric fatty acid | cationic | 1 |
| compound of the invention | conditioning | |
| | agent | |
| Phase B | | |
| BeauSil Gel 8017 (CHT) (C13-15 alkane, | dimethicone/ | 70 |
| isododecane and | vinyldimethicone | |
| dimethicone/vinyldimethicone | crosspolyme | |
| crosspolymer and caprylyl methicone) | | |
| Phase C | | |
| BeauSI Wax 070 (CHT)(Cetyl | | 0.6 |
| dimethicone) | | |
| Argan Oil (Argania spinose kernel oil) | | 0.2 |
| Vitamin E (Tocopherol) | | 0.05 |
| Fragrance | | q.s. |

### Procedure

Ingredients of phase B were added to phase A and mixed with low to medium shear. The ingredients of phase C were blended and added to phase AB.

### EXAMPLES

(The percentages refer to weight-% unless otherwise indicated).

As used herein, the term “castor oil” generally refers to ricinoleic acid triglyceride).

### Remarks on the Nomenclature Used Herein for Estolide Moieties and Estolide Compounds

In the nomenclature for denoting the structure of the estolide groups as used in the following examples, which refers to the compounds from which the estolide moieties are at least formally obtained by esterification, the carboxylic acids from which the estolide moieties are at least formally derived are given in a sequential manner in parentheses. In case there are several subunits derived from the same acid in a row present in the estolide moiety and these are indicated in parentheses, wherein a subscript integer indicates the number of repeating units, the carboxylic acids are given in brackets.

It is noted that the specific carboxylic acids given in parentheses or brackets are not combined in a random structure, but they have exactly the sequence of hydroxyl-carboxylic acid-derived residues and carboxylic acid-derived residues, respectively, as indicated in the term used. Therein, the last carboxylic acid given in the term in parentheses or brackets, respectively, is the terminal carboxylic acid of the estolide moiety. Going from the beginning of the term in parentheses or bracktes to the end of the term, the order of carboxylic acid residues linked by ester groups is displayed in the correct order and number of residues contained.

For example, the term “(12-hydroxy stearic acid-ricinoleic acid-oleic acid)” refers to an estolide moiety in which formally 12-hydroxy stearic acid molecule is linked via its OH group to the carboxylic acid group of a ricinoleic acid molecule by forming an ester group. The hydroxyl group of the said ricinoleic acid group is linked to an oleic acid molecule by forming an ester group with the carboxylic acid group of the oleic acid molecule. The oleic acid is in this example considered to be the terminal group of this specific estolide moiety, as, if the estolide moiety is a substituent of a higher-level structure (i.e. a more complex molecule), in general the estolide moiety is linked to the overall structure via linkage to the carboxylic acid group of the first mentioned residue of the term used for the estolide moiety. In this case, this is the first mentioned 12-hydroxy stearic acid residue, and the oleic acid residue is the terminal group of the estolide moiety.

Accordingly, in case the term used refers to a carboxylic acid chloride of an estolide structure, the acyl chloride group is necessarily formed from the carboxylic acid group of the first-mentioned carboxylic acid residue in parentheses, i.e. the most remote one from the terminal group.

In case terms as “dimer” or “trimer” and so on are used, this refers to the number of carboxylic acid-derived subunits of the estolide moieties.

In the same manner, the term “[(ricinoleic acid)₂-oleic acid] estolide” refers to an estolide moiety or compound in which formally a ricinoleic acid molecule or residue is linked via its OH group to the carboxylic acid group of a further ricinoleic acid molecule by forming an ester group. The hydroxyl group of the latter ricinoleic acid group mentioned is linked to an oleic acid molecule by forming an ester group with the carboxylic acid group of the oleic acid molecule. The oleic acid is considered to be the terminal group of this specific estolide moiety, as, if the estolide moiety is a substituent of a higher-level structure (i.e. a more complex molecule), the estolide moiety is linked to the overall structure via linkage to the carboxylic acid group of the first mentioned ricinoleic acid residue, and the oleic acid residue is the terminal group of the estolide moiety.

In the case estolide moieties are linked by a linking group via ester or amide groups, such as by the succinic acid derived residue in [(ricinoleic acid)₆-succinic acid-(ricinoleic acid)₆], linked to two ricinoleic acid estolide groups by an ester group on each side, this is indicated by incorporation of the name of the parent compound into the term applied to the overall estolide structure. Thus a comprehensive term indicating the sequence of carboxylic acid residues is provided.

It is further noted that the exact structure of the estolides is primarily clarified by the structural formulas, which are thoroughly provided for the example compounds, and that the structures of the example compounds can also be clearly derived by the skilled artisan from the detailed experimental procedures provided.

The term “commercial polyglycerol-polyricinolate” refers to Palsgaard® PGPR 4150, a commercially available polyglycerine-polyricinoleate produced by Palsgaard A/S, which is specified as followed:

Polyglycerol-polyricinoleate (E476) present as a yellowish, viscous liquid; viscosity reducing power: 74-87; max. acid number: 3 mg KOH/g; hydroxyl number: 80-100 mg KOH/g; refractive index at 65° C.: 1.4630-1.4665; iodine number: 72-103 g l₂/100 g; saponification number: 170-210 mg KOH/g; polyglycerol-composition: di-, tri- and tetraglycerine, at least: 75%; polyclycerol equal or longer than heptaglycerol, max.: 10%.

### Synthesis Example 1

### Synthesis of a (Ricinoleic Acid-Oleic Acid) Estolide Dimer

In a 1000 ml four-necked bottle, equipped with refluxing condenser, thermometer and magnetic stirrer, dropping funnel and gas outlet tube 225 g (0.75 mol) ricinoleic acid were placed at room temperature under a nitrogen atmosphere. Upon stirring, 226.85 g (0.75 mol) oleic acid chloride were added slowly during 1.5 h. The temperature increased from 22 to 32° C. The temperature increase was accompanied by the generation of gas bubbles indicating the formation of HCl. The temperature was maintained at 32° C. for further 2 h, afterwards it was increased to 50° C. and was maintained there for 1 h. Volatiles were removed under reduced pressure (40° C./2 h/20 mmHg). The conversion of the OH groups was determined by means of ¹H NMR spectroscopy. The conversion of the OH groups was 100%.

A brownish, transparent oil essentially having the following structure was obtained:

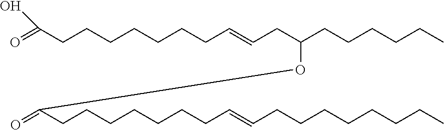 [SMILES: CCCCCCCC/C=C/CCCCCCCC(=O)OC(C/C=C/CCCCCCCC(=O)O)CCCCCC]

### Synthesis Example 2

### Synthesis of a [(Ricinoleic Acid)₂-Oleic Acid] Estolide Trimer

In a 250 ml four-necked bottle, equipped with refluxing condenser, thermometer and magnetic stirrer, dropping funnel and gas outlet tube 34.87 g (0.293 mol) SOCl₂ were placed at room temperature under a nitrogen atmosphere. Upon stirring, 110 g (0.195 mol) of the estolide dimer of synthesis example 1 were added slowly during 1 h. After the end of the addition the temperature was increased to 80° C. The temperature was maintained at 80° C. for 1 h. Volatiles were removed under reduced pressure (80° C./2 h/20 mmHg). Nitrogen was used to break the vacuum and 57.75 g (0.195 mol) ricinoleic acid were added to the carboxylic acid chloride intermediate at 80° C. over 45 minutes. The temperature was maintained for 2 h. Volatiles were removed under reduced pressure (40° C./2 h/20 mmHg). The conversion of the OH groups was determined by means of ¹H NMR spectroscopy. The conversion of the OH groups was 100%.

A brownish, transparent oil essentially having the following structure was obtained:

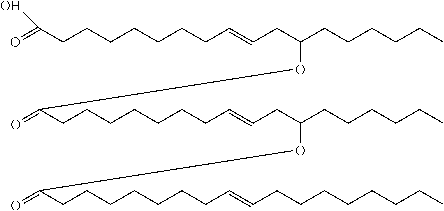 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

### Synthesis Example 2a

### Synthesis of a [(Ricinoleic Acid)₂-Stearic Acid] Estolide Trimer

Two 250 ml three-necked bottles A and B, equipped with refluxing condenser, thermometer and magnetic stirrer, dropping funnel and gas outlet tube were flushed with nitrogen.

Bottle A was used to react fatty acid chlorides with ricinoleic acid yielding a chain extended fatty ester acid. Subsequent addition of SOCl₂ yielded the corresponding fatty ester acid chloride.

Bottle B was used to react the formed fatty ester acid chloride with ricinoleic acid yielding a chain extended fatty ester acid. Subsequent addition of SOCl₂ yielded the corresponding fatty ester acid chloride. This fatty acid chloride was transferred back to bottle A and reacted with fresh ricinoleic acid. The above described cycle may be repeated until the hexamer estolide [(ricinoleic acid)₅-stearic acid] is prepared.

General procedure for the synthesis of chain extended fatty ester acids: The calculated amount of ricinoleic acid was placed in a bottle. An equimolar amount of fatty ester acid chloride was added slowly at room temperature. In order to complete the reaction, the temperature was increased to 80° C. for 3 h. The complete conversion of the OH groups was determined by means of ¹H NMR spectroscopy.

General procedure for the synthesis of fatty ester acid chlorides: The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups to C(O)CL groups was determined by means of ¹H NMR spectroscopy.

The following table summarizes the materials and the quantities used.

| | | fatty ester | fatty acid and | SOCl₂ | |
|---|---|---|---|---|---|
| | | derivative | amount of fatty | amount | |
| bottle | fatty ester derivative | amount [g] | acid [g] | [g] | target product |
| A | stearic acid chloride | 55.83 | ricinoleic acid | | (rici-stearic acid) |
| | | | 55.00 | | |
| A | (rici-stearic acid) | 104.12 | | 32.9 | (rici-stearic acid) |
| | | | | | chloride |
| B | (rici-stearic acid) | 103.85 | ricinoleic acid | | [(rici)₂-stearic acid] |
| | chloride | | 53.13 | | |

Note:
The term “rici” replaces the term “ricinoleic acid” in denoting a ricinoleyl radical.

The formula of [(rici)₂-stearic acid] is as follows:

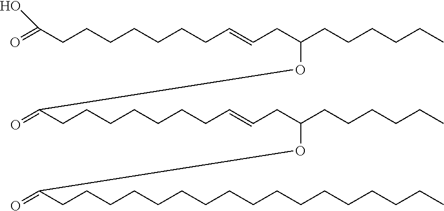 [SMILES: CCCCCCCCCCCCCCCCCC(=O)OC(C/C=C/CCCCCCCC(=O)OC(C/C=C/CCCCCCCC(=O)O)CCCCCC)CCCCCC]

### Synthesis Example 2b

### Synthesis of a (Ricinoleic Acid-12-Hydroxy Stearic Acid-Oleic Acid) Estolide Trimer

The procedure outlined for synthesis example 2a was repeated.

The following table summarizes the materials and the quantities used.

| | | fatty ester | fatty acid and | SOCl₂ | |
|---|---|---|---|---|---|
| | | derivative | amount of fatty | amount | |
| bottle | fatty ester derivative | amount [g] | acid [g] | [g] | target product |
| A | oleic acid chloride | 55.37 | 12-hydroxy | | (12-hydroxy stea- |
| | | | stearic acid | | oleic acid) |
| | | | 55.3 | | |
| A | (12-hydroxy stea- | 103.27 | | 39.8 | (12-hydroxy stea- |
| | oleic acid) | | | | oleic acid) chloride |
| B | (12-hydroxy stea- | 104.8 | ricinoleic acid | | (rici - 12 hydroxy |
| | oleic acid) chloride | | 53.61 | | stea- oleyl acid) |

Note:
The term “rici” replaces the term “ricinoleic acid” in denoting a ricinoleyl radical, the term “12-hydroxy-stea” replaces the term “12-hydroxy stearic acid” in denoting a 12-hydroxyl stearyl radical.

The formula of rici-(12 hydroxy stea)-oleic acid is as follows:

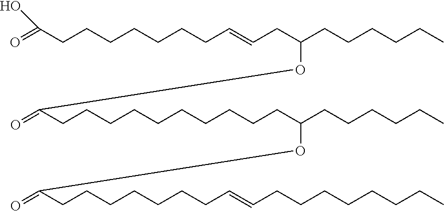 [SMILES: CCCCCCCC/C=C/CCCCCCCC(=O)OC(CCCCCC)CCCCCCCCCCC(=O)OC(C/C=C/CCCCCCCC(=O)O)CCCCCC]

### Synthesis Example 2c

### Synthesis of a (12-Hydroxy Stearic Acid-Ricinoleic Acid-Oleic Acid) Estolide Trimer

The procedure outlined for synthesis example 2a was repeated.

The following table summarizes the materials and the quantities used.

| | | fatty ester | fatty acid and | SOCl₂ | |
|---|---|---|---|---|---|
| | | derivative | amount of fatty | amount | |
| bottle | fatty ester derivative | amount [g] | acid [g] | [g] | target product |
| A | oleic acid chloride | 55.00 | ricinoleic acid | | (rici - oleic acid) |
| | | | 55.3 | | |
| A | (rici - oleic acid) | 102.91 | | 38.5 | (rici - oleic acid) |
| | | | | | chloride |
| B | (rici - oleic acid) | 100.00 | 12-hydroxy | | (12-hydroxy stea - |
| | chloride | | stearic acid | | rici - oleic acid) |
| | | | 51.68 | | |

Note:
The term “rici” replaces the term “ricinoleic acid” in denoting a ricinoleyl radical, the term “12-hydroxy-stea” replaces the term “12-hydroxy stearic acid” in denoting a 12-hydroxyl stearyl radical.

The formula of (12-hydroxy stea-rici-oleic acid) is as follows:

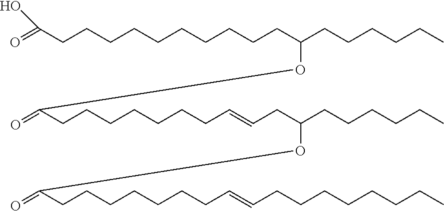 [SMILES: CCCCCCCC/C=C/CCCCCCCC(=O)OC(C/C=C/CCCCCCCC(=O)OC(CCCCCC)CCCCCCCCCCC(=O)O)CCCCCC]

### Synthesis Example 3

### Synthesis of a [(Ricinoleic Acid)s-Oleic Acid] Estolide Hexamer and the Corresponding [(Ricinoleic Acid)s-Oleic Acid] Chloride Hexamer

Two 100 ml three-necked bottles A and B, equipped with refluxing condenser, thermometer and magnetic stirrer, dropping funnel and gas outlet tube were flushed with nitrogen.

Bottle A was used to react fatty acid chlorides with ricinoleic acid, yielding a chain extended fatty ester acid. Subsequent addition of SOCl₂ yielded the corresponding fatty ester acid chloride.

Bottle B was used to react the formed fatty ester acid chloride with ricinoleic acid, yielding a chain extended fatty ester acid. Subsequent addition of SOCl₂ yielded the corresponding fatty ester acid chloride. This fatty acid chloride was transferred back to bottle A and reacted with fresh ricinoleic acid. The above described cycle was repeated until the hexamer estolide [(ricinoleic acid)₅-oleic acid] was prepared.

### General Procedure for the Synthesis of Chain Extended Fatty Ester Acids:

The calculated amount of ricinoleic acid as placed in a bottle. An equimolar amount of fatty ester acid chloride was added slowly at room temperature. In order to complete the reaction, the temperature was increased to 80° C. for 3 h. The complete conversion of the OH groups of the ricinoleic acid into ester was determined by means of ¹H NMR spectroscopy.

### General Procedure for the Synthesis of Fatty Ester Acid Chlorides:

The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)CL groups was determined by means of ¹H NMR spectroscopy.

The following table summarizes the materials and the quantities used.

| | | fatty ester | | SOCl₂ | |
|---|---|---|---|---|---|
| | | derivative | ricinoleic acid | amount | |
| bottle | fatty ester derivative | amount [g] | amount [g] | [g] | target product |
| A | oleic chloride | 30.00 | 29.76 | | [(rici)₁-oleic acid] |
| A | [(rici)₁-oleic acid] | 59.76 | | 37.89 | [(rici)₁-oleic acid] |
| | | | | | chloride |
| B | [(rici)₁-oleic acid] | 46.26 | 23.75 | | [(rici)₂-oleic acid] |
| | chloride | | | | |
| B | [(rici)₂-oleic acid] | 70.01 | | 29.63 | [(rici)₂-oleic acid] |
| | | | | | chloride |
| A | [(rici)₂-oleic acid] | 52.00 | 18.01 | | [(rici)₃-oleic acid] |
| | chloride | | | | |
| A | [(rici)₃-oleic acid] | 70.01 | | 22.23 | [(rici)₃-oleic acid] |
| | | | | | chloride |
| B | [(rici)₃-oleic acid] | 55.50 | 14.50 | | [(rici)₄-oleic acid] |
| | chloride | | | | |
| B | [(rici)₄-oleic acid] | 70.00 | | 17.79 | [(rici)₄-oleic acid] |
| | | | | | chloride |
| A | [(rici)₄-oleic acid] | 57.87 | 12.14 | | [(rici)₅-oleic acid] |
| | chloride | | | | |
| A | [(rici)₅-oleic acid | 50 | | 10.60 | [(rici)₅-oleic acid] |
| | | | | | chloride |

Note:
The term “rici” replaces the term “ricinoleic acid” in denoting a ricinoleyl radical, the term “12-hydroxyl-stea” replaces the term “12-hydroxy stearic acid” in denoting a 12-hydroxyl stearyl radical.

A brownish, transparent oil essentially having the following structure [(rici)₅-oleic acid] was obtained:

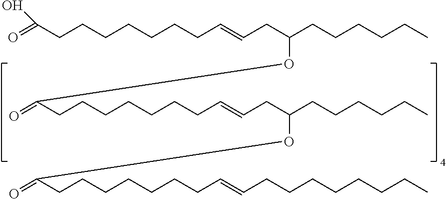

The corresponding [(rici)₅-oleic acid] chloride has the structure:

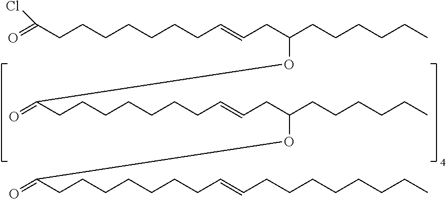

### Synthesis Example 4

### Synthesis of a Branched Bis-[(Ricinoleic Acid)₂-Oleic Acid] Estolide Based on Bis 2,2-Hydroxymethyl Propionic Acid

In a 100 ml three-necked bottle, equipped with refluxing condenser, thermometer and magnetic stirrer, dropping funnel and gas outlet tube 48.88 g (0.0567 mol) of the [(ricinoleic acid)₂-oleic acid] chloride of synthesis example 3 were mixed with 3.80 g (0.0284 mol) bis 2,2-hydroxymethyl propionic acid. The mixture was heated to 100° C. for 8 h. Volatiles were removed under reduced pressure (80° C./1 h/20 mmHg). The complete conversion of the OH groups of 2,2-hydroxymethyl propionic acid was determined by means of ¹H NMR spectroscopy.

A brownish, transparent oil essentially having the following structure was obtained:

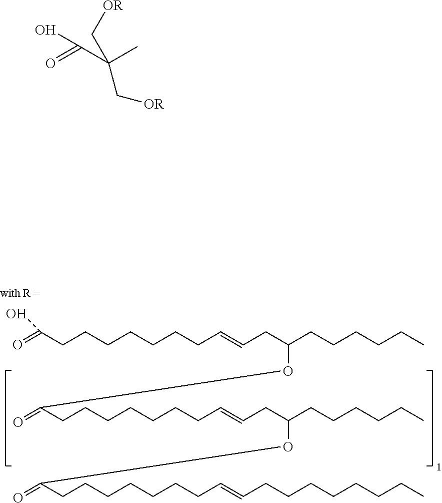

### Synthesis Example 4a

### Synthesis of a Dendrimeric Bis-[(Ricinoleic Acid)₂-Oleic Acid] Estolide

In a 100 ml three-necked bottle, equipped with refluxing condenser, thermometer and magnetic stirrer, dropping funnel and gas outlet tube 64.57 g (0.03617 mol) of the branched bis-[(ricinoleic acid)₂-oleic acid] estolide based on bis 2,2-hydroxymethyl propionic acid of synthesis example 4 were heated 80° C. 8.61 g (0.0723 mol) SOCl₂ were added within 10 minutes. The reaction is maintained for 4 h. Volatiles were removed under reduced pressure (80° C./1 h/20 mmHg). The complete conversion of the C(O)OH groups to C(O)CL groups was determined by means of ¹H NMR spectroscopy. 2.42 g (0.01808 mol) bis-2,2-hydroxymethyl propionic acid were added at 80° C. and the reaction was maintained for additional 5 h. Volatiles were removed under reduced pressure (80° C./0.5 h/20 mmHg). The complete conversion of the terminal OH groups of the bis-2,2-hydroxymethyl propionic acid was determined by means of ¹H NMR spectroscopy.

A viscous brownish, transparent oil essentially having the following dendrimeric structure was obtained:

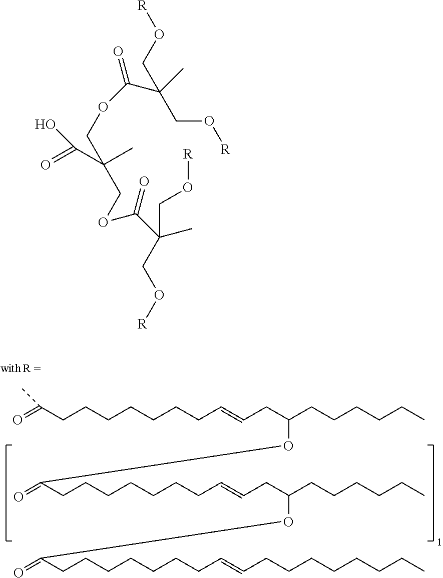

### Synthesis Example 4b

### Synthesis of a Branched Bis-[(Ricinoleic Acid)₂-Stearic Acid] Estolide Based on Bis 2,2-Hydroxymethyl Propionic Acid

In a 250 ml three-necked bottle, equipped with refluxing condenser, thermometer and magnetic stirrer, dropping funnel and gas outlet tube 95.06 g (0.1124 mol) of the [(ricinoleic acid)₂-stearic acid] estolide of synthesis example 2a were heated to 80° C. 33.8 g (0.28 mol) SOCl₂ were added within 10 minutes. The reaction was maintained for 4 h. Volatiles are removed under reduced pressure (80° C./1 h/20 mmHg). The complete conversion of the C(O)OH groups towards C(O)CL groups was determined by means of ¹H NMR spectroscopy.

In a 250 ml three-necked bottle, equipped with refluxing condenser, thermometer and mechanical stirrer, dropping funnel and gas outlet tube 92.88 g (0.1075 mol) of the (ricinoleic acid)₂-stearic acid chloride intermediate and 7.22 g (0.0538 mol) bis 2,2-hydroxymethyl propionic acid were mixed at 80° C. and the reaction was maintained for additional 5 h. Volatiles were removed under reduced pressure (80° C./0.5 h/20 mmHg). The complete conversion of the OH groups of the bis 2,2-hydroxymethyl propionic acid was determined by means of ¹H NMR spectroscopy.

A viscous brownish, transparent oil essentially having the following structure was obtained:

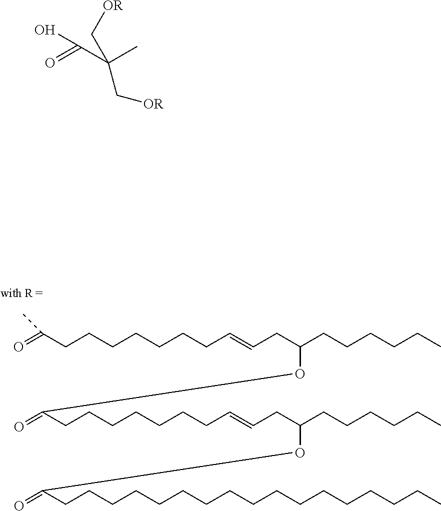

### Synthesis Example 4c

### Synthesis of a Branched Bis-(Ricinoleic Acid-12-Hydroxy Stearic Acid-Oleic Acid) Estolide Based on Bis 2,2-Hydroxymethyl Propionic Acid

In a 250 ml three-necked bottle, equipped with refluxing condenser, thermometer and magnetic stirrer, dropping funnel and gas outlet tube 102.95 g (0.1218 mol) of the (ricinoleic acid-12 hydroxy stearic acid-oleic acid) estolide of synthesis example 2b were heated to 80° C. 42.8 g (0.36 mol) SOCl₂ were added within 10 minutes. The reaction was maintained for 4 h. Volatiles were removed under reduced pressure (80° C./1 h/20 mmHg). The complete conversion of the C(O)OH groups to C(O)CL groups was determined by means of ¹H NMR spectroscopy.

In a 250 ml three-necked bottle, equipped with refluxing condenser, thermometer and mechanical stirrer, dropping funnel and gas outlet tube 104.22 g (0.1206 mol) of the bis-(ricinoleic acid-12 hydroxy stearic acid-oleic acid) chloride intermediate and 8.09 g (0.0603 mol) bis-2,2-hydroxymethyl propionic acid were mixed at 80° C. and the reaction was maintained for additional 5 h. Volatiles were removed under reduced pressure (80° C./0.5 h/20 mmHg). The complete conversion of the OH groups from bis 2,2-hydroxymethyl propionic acid was determined by means of ¹H NMR spectroscopy.

A viscous brownish, transparent oil essentially having the following structure was obtained:

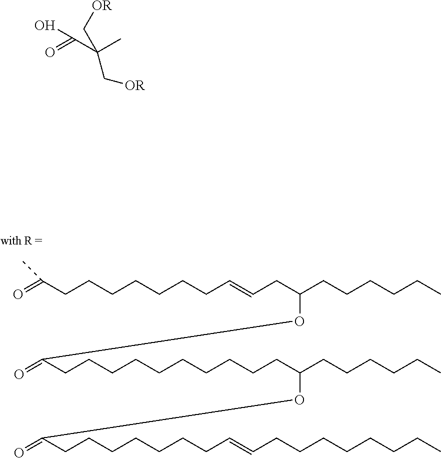

### Synthesis Example 4d

### Synthesis of a Branched (12-Hydroxy Stearic Acid-Ricinoleic Acid-Oleic Acid) Estolide Based on Bis 2,2-Hydroxymethyl Propionic Acid

In a 250 ml three-necked bottle, equipped with refluxing condenser, thermometer and magnetic stirrer, dropping funnel and gas outlet tube 98.05 g (0.116 mol) of the (12-hydroxy stearic acid-ricinoleic acid-oleic acid) estolide of synthesis example 2c were heated to 80° C. 30.08 g (0.25 mol) SOCl₂ were added within 10 minutes. The reaction was maintained for 4 h. Volatiles were removed under reduced pressure (80° C./1 h/20 mmHg). The complete conversion of the C(O)OH groups to C(O)CL groups was determined by means of ¹H NMR spectroscopy.

In a 250 ml three-necked bottle, equipped with refluxing condenser, thermometer and mechanical stirrer, dropping funnel and gas outlet tube 97.4 g (0.1127 mol) of the (12-hydroxy stearic acid-ricinoleic acid-oleic acid) chloride intermediate and 7.56 g (0.0564 mol) bis 2,2-hydroxymethyl propionic acid were mixed at 80° C. and the reaction was maintained for further 5 h. Volatiles were removed under reduced pressure (80° C./0.5 h/20 mmHg). The complete conversion of the OH groups of the hydroxymethyl groups was determined by means of ¹H NMR spectroscopy.

A viscous brownish, transparent oil essentially having the following structure was obtained:

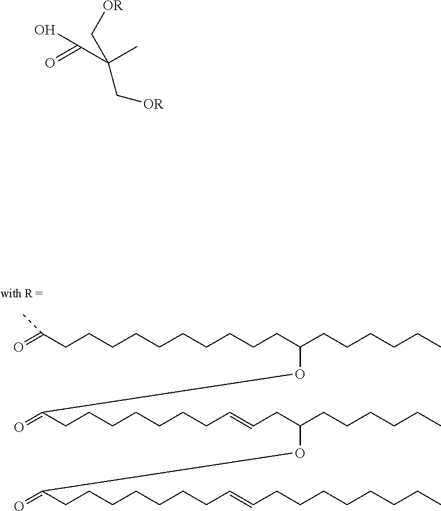

### Synthesis Example 5

### Synthesis of an Alpha Branched Bis-[(Ricinoleic Acid)₅-Oleic Acid] Estolide Based on Bis 2,2-Hydroxymethyl Propionic Acid

In a 100 ml three-necked bottle, equipped with refluxing condenser, thermometer and magnetic stirrer, dropping funnel and gas outlet tube 29.53 g (0.0173 mol) of the [(ricinoleic acid)₅-oleic acid] chloride of synthesis example 3 were mixed with 1.16 g (0.00866 mol) bis 2,2-hydroxymethyl propionic acid. The mixture was heated to 105° C. for 5 h. Volatiles were removed under reduced pressure (80° C./10 min/20 mmHg). The complete conversion of the OH groups of bis 2,2-hydroxymethyl propionic acid was determined by means of ¹H NMR spectroscopy.

A brownish, transparent oil essentially having the following structure was obtained:

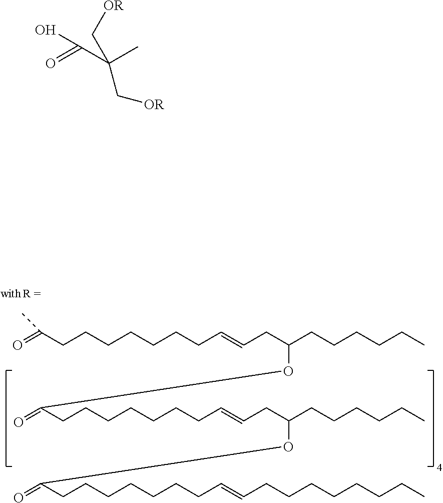

### Synthesis Example 6

### Synthesis of a Chloro Acetic Acid Ester Derivative of a Glycerol Based Estolide

In a 100 ml three-necked bottle, equipped with refluxing condenser, thermometer, mechanical stirrer, dropping funnel and gas outlet tube 40 g (0.434 mol) glycerol were placed at room temperature. Upon stirring, 49.05 g (0.434 mol) chloro acetic acid chloride were added over 45 minutes. The temperature increased during the addition to 83° C. Afterwards, the temperature was increased to 120° C. for 2 h. The formation of the chloro acetic acid ester was confirmed by means of ¹H NMR spectroscopy.

5.72 g (0.034 mol) of the glycerol mono chloro acetate were mixed at room temperature with 39.50 g (0.068 mol) of the [(ricinoleic acid)-oleic acid] chloride from synthesis example 3. The mixture was heated to 100° C. for 8 h. Volatiles were removed under reduced pressure (80° C./1 h/20 mmHg). The complete conversion of the OH groups and the formation of additional ester moieties was confirmed by means of ¹H NMR spectroscopy.

A brownish, transparent oil essentially having the approximate structure was obtained:

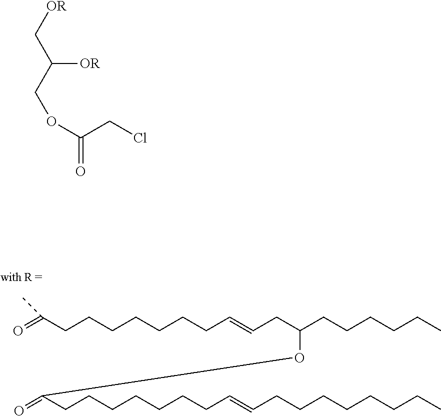

### Synthesis Example 7

### Synthesis of a Chloro Acetic Acid Ester Derivative of a Castor Oil Based Estolide

In a 100 ml three-necked bottle, equipped with refluxing condenser, thermometer, mechanical stirrer, dropping funnel and gas outlet tube 40 g (0.0428 mol) castor oil were placed at room temperature. Upon stirring, 4.84 g (0.0428 mol) chloro acetic acid chloride were added over 10 minutes. The temperature increased during the addition to 34° C. Afterwards, the temperature was increased to 80° C. for 3 h. The formation of the chloro acetic acid ester was confirmed by means of ¹H NMR spectroscopy.

49.83 g (0.0856 mol) of the [(ricinoleic acid)-oleic acid] chloride from synthesis example 3 were added. The temperature was maintained at 80° C. for 8 h. Volatiles were removed under reduced pressure (80° C./2 h/20 mmHg). The complete conversion of the OH groups of the castor oil molecule and the formation of additional ester moieties was confirmed by means of ¹H NMR spectroscopy.

A brownish, transparent oil essentially having the approximate structure was obtained:

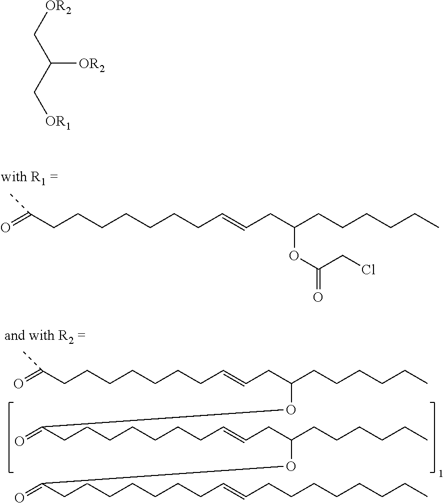

### Synthesis Example 7a

### Synthesis of a Chloro Acetic Acid Ester Derivative of a Castor Oil Based Estolide

In a 250 ml three-necked bottle, equipped with refluxing condenser, thermometer, mechanical stirrer, dropping funnel and gas outlet tube 80 g (0.0857 mol) castor oil were placed and heated to 60° C. Upon stirring, 9.68 g (0.0857 mol) chloro acetic acid chloride were added over 10 minutes. The temperature increased during the addition to 80° C. Afterwards, the temperature was maintained at 80° C. for additional 1.5 hrs. The formation of the chloro acetic acid ester was confirmed by means of ¹H NMR spectroscopy.

100 g (0.1714 mol) of the [(ricinoleic acid)₁-stearic acid] chloride from synthesis example 2a were added. The temperature was maintained at 80° C. for 4 h. Volatiles were removed under reduced pressure (90° C./2 h/20 mmHg). The complete conversion of the OH groups of the castor oil molecule and the formation of additional ester moieties was confirmed by means of ¹H NMR spectroscopy.

A brownish wax like material essentially having the approximate structure was obtained:

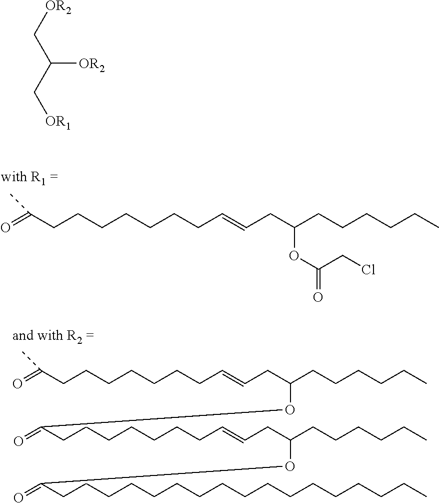

### Example 1

Synthesis of an Amine Salt of a Tertiary Amino Alcohol Ester of the [(Ricinoleic Acid)₂-Oleic Acid] Estolide

In a 500 ml four-necked bottle, equipped with refluxing condenser, thermometer and mechanic stirrer, dropping funnel and gas outlet tube 300 ml n-heptane were mixed at room temperature with 49.69 g (0.0577 mol) of the [(ricinoleic acid)₂-oleic acid] chloride of synthesis example 3. 5.95 g (0.0577 mol) of (CH₃)₂NCH₂CH₂CH₂OH were added over 20 minutes. The temperature increased to 37° C. The temperature was maintained for 30 minutes. The n-heptane was removed under reduced pressure (30° C./2 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

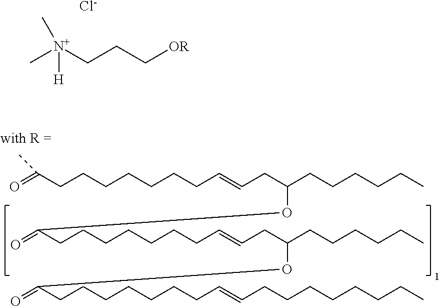

### Example 2

Synthesis of an Amine Salt of a Tertiary Amino Alcohol Ester of the [(Ricinoleic Acid)₅-Oleic Acid] Estolide

In a 250 ml four-necked bottle, equipped with refluxing condenser, thermometer and mechanic stirrer, dropping funnel and gas outlet tube 40 ml n-heptane were mixed at 40° C. with 49.19 g (0.0289 mol) of the (ricinoleic acid)₅-oleoyl chloride of synthesis example 3. 2.89 g (0.0289 mol) of (CH₃)₂NCH₂CH₂CH₂OH were added over 5 minutes. The temperature increased to 43° C.

The temperature was maintained for 1 h. The n-heptane was removed under reduced pressure (30° C./2 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

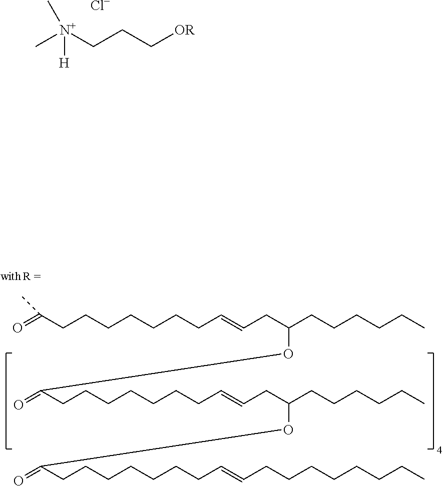

### Example 3

Synthesis of an Amine Salt of a Tertiary Amino Alcohol Ester of the Branched Bis-[(Ricinoleic Acid)₂-Oleic Acid] Estolide

In a 100 ml three-necked bottle, equipped with refluxing condenser, thermometer, magnetic stirrer, dropping funnel and gas outlet tube 25 g (0.014 mol) of the alpha branched bis-[(ricinoleic acid)₂-oleic acid] estolide of synthesis example 4 were mixed at 50° C. with 11 g (0.092 mol) SOCl₂. The mixture was heated to 80° C. for 2.5 h. Afterwards, the excess of SOCl₂ was removed under reduced pressure (80° C./1 h/20 mmHg). The formation of the acid chloride was confirmed by means of ¹H NMR spectroscopy. 22.4 g (0.0124 mol) of the acid chloride were transferred into a 250 ml four necked bottle, equipped with refluxing condenser, thermometer, mechanical stirrer and dropping funnel. 140 ml n-heptane and 1.28 g (0.00825 mol) of (CH₃)₂NCH₂CH₂CH₂OH were added over 5 minutes. The temperature increased to 28° C. and was maintained for 1 h. The n-heptane was removed under reduced pressure (30° C./3 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

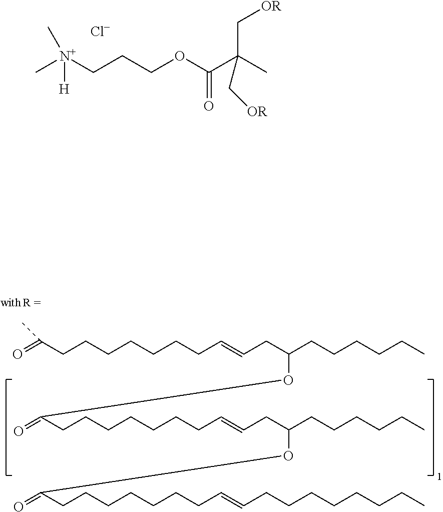

### Example 3a

### Synthesis of an Amine Salt of a Tertiary Amino Alcohol Ester of a Dendrimeric Bis-[(Ricinoleic Acid)₂-Oleic Acid] Estolide

In a 100 ml three-necked bottle, equipped with refluxing condenser, thermometer, magnetic stirrer, dropping funnel and gas outlet tube 25 g (0.014 mol) of the branched bis-[(ricinoleic acid)₂-oleic acid] estolide of synthesis example 4a were mixed at 50° C. with 11 g (0.092 mol) SOCl₂. The mixture was heated to 80° C. for 2.5 h. Afterwards, the excess of SOCl₂ was removed under reduced pressure (80° C./1 h/20 mmHg). The formation of the acid chloride was confirmed by means of ¹H NMR spectroscopy.

22.4 g (0.0124 mol) of the acid chloride were transferred into a 250 ml four necked bottle, equipped with refluxing condenser, thermometer, mechanical stirrer and dropping funnel. 140 ml n-heptane and 1.28 g (0.00825 mol) of (CH₃)₂NCH₂CH₂CH₂OH were added during 5 minutes. The temperature increased to 28° C. and was maintained for 1 h. The n-heptane was removed under reduced pressure (30° C./3 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

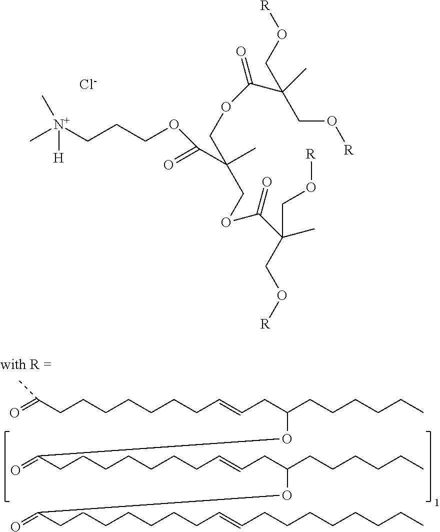

### Example 3b

### Synthesis of an Amine Salt of a Tertiary Amino Alcohol Ester of the Alpha Branched Bis-[(Ricinoleic Acid)₂- Stearic Acid] Estolide

In a 250 ml three-necked bottle, equipped with refluxing condenser, thermometer, magnetic stirrer, dropping funnel and gas outlet tube 77 g (0.0430 mol) of the alpha branched bis-[(ricinoleic acid)₂-stearic acid] estolide of synthesis example 4b were mixed at 50° C. with 34.5 g (0.29 mol) SOCl₂. The mixture was heated to 80° C. for 2.5 h. Afterwards, the excess of SOCl₂ was removed under reduced pressure (80° C./1 h/20 mmHg). The formation of the acid chloride was confirmed by means of ¹H NMR spectroscopy.

71.5 g (0.0396 mol) of the acid chloride were transferred into a 500 ml four necked bottle, equipped with refluxing condenser, thermometer, mechanical stirrer and dropping funnel. 190 g n-heptane and 4.09 g (0.0396 mol) of (CH₃)₂NCH₂CH₂CH₂OH were added over 5 minutes. The temperature increased to 30° C. and was maintained for 1 h. The n-heptane was removed under reduced pressure (30° C./3 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

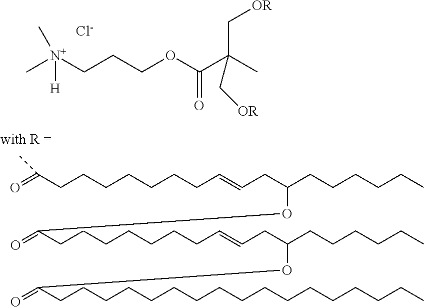

### Example 3c

### Synthesis of an Amine Salt of a Tertiary Amino Alcohol Ester of the Alpha Branched Bis-(Ricinoleic Acid-12 Hydroxy Stearic Acid-Oleic Acid) Estolide

In a 250 ml three-necked bottle, equipped with refluxing condenser, thermometer, magnetic stirrer, dropping funnel and gas outlet tube, 89.4 g (0.05 mol) of the alpha branched bis-(ricinoleic acid-12 hydroxy stearic acid-oleic acid) estolide of synthesis example 4c were mixed at 50° C. with 40.4 g (0.34 mol) SOCl₂. The mixture was heated to 80° C. for 2.5 h. Afterwards, the excess of SOCl₂ was removed under reduced pressure (80° C./1 h/20 mmHg). The formation of the acid chloride was confirmed by means of ¹H NMR spectroscopy.

85.11 g (0.0471 mol) of the said acid chloride were transferred into a 500 ml four necked bottle, equipped with refluxing condenser, thermometer, mechanical stirrer and dropping funnel. 187 g n-heptane and 4.86 g (0.0471 mol) of (CH₃)₂NCH₂CH₂CH₂OH were added over 5 minutes. The temperature increased to 32° C. and was maintained for 1 h. The n-heptane was removed under reduced pressure (30° C./3 h/20 mmHg). The conversion of the OH groups of (CH₃)₂NCH₂CH₂CH₂OH and the formation of the ester was confirmed by means of ¹H NMR spectroscopy.

A brownish wax having the following structure was obtained:

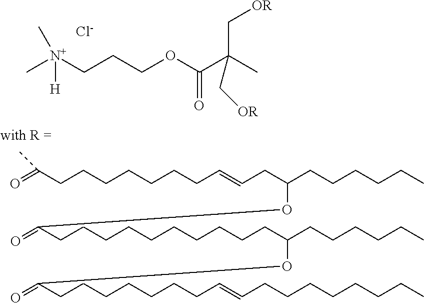

### Example 3d

### Synthesis of an Amine Salt of a Tertiary Amino Alcohol Ester of the Alpha Branched Bis-(12-Hydroxy Stearic Acid-Ricinoleic Acid-Oleic Acid) Estolide

In a 250 ml three-necked bottle, equipped with refluxing condenser, thermometer, magnetic stirrer, dropping funnel and gas outlet tube 78 g (0.0436 mol) of the alpha branched bis-(12 hydroxy stearic acid-ricinoleic acid-oleic acid) estolide of synthesis example 4d were mixed at 50° C. with 22.77 g (0.19 mol) SOCl₂. The mixture was heated to 80° C. for 2.5 h. Afterwards, the excess of SOCl₂ was removed under reduced pressure (80° C./1 h/20 mmHg). The formation of the acid chloride was confirmed by means of ¹H NMR spectroscopy.

76.15 g (0.0421 mol) of the acid chloride were transferred into a 500 ml four necked bottle, equipped with refluxing condenser, thermometer, mechanical stirrer and dropping funnel. 200 g n-heptane and 4.34 g (0.0421 mol) (CH₃)₂NCH₂CH₂CH₂OH were added over 5 minutes. The temperature increased to 28° C. and was maintained for 1 h. The n-heptane was removed under reduced pressure (30° C./3 h/20 mmHg). The conversion of the OH groups and the formation of the ester was confirmed by means of ¹H NMR spectroscopy.

A brownish wax having the following structure was obtained:

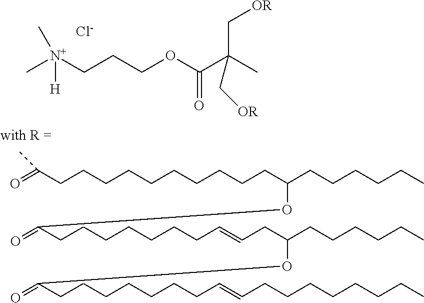

### Example 4

### Synthesis of an Amine Salt of a Tertiary Amino Alcohol Ester of the Alpha Branched Bis-[(Ricinoleic Acid)₅-Oleic Acid] Estolide

In a 100 ml three-necked bottle, equipped with refluxing condenser, thermometer, magnetic stirrer, dropping funnel and gas outlet tube 28.63 g (0.00825 mol) of the alpha branched bis-[(ricinoleic acid)₅-oleic acid] estolide of synthesis example 5 were mixed at room temperature with 7.2 g (0.0605 mol) SOCl₂. The mixture was heated to 80° C. for 2.5 h. Afterwards, the excess of SOCl₂ was removed under reduced pressure (80° C./1 h/20 mmHg). The formation of thus formed acid chloride was confirmed by means of ¹H NMR spectroscopy.

The temperature was adjusted to 45° C. and 40 ml n-heptane as well as 0.85 g (0.00825 mol) of (CH₃)₂NCH₂CH₂CH₂OH were added over 5 minutes. The temperature was maintained for 1 h. The n-heptane was removed under reduced pressure (30° C./3 h/20 mmHg). The conversion of the OH groups of (CH₃)₂NCH₂CH₂CH₂OH and the formation of the ester was confirmed by means of ¹H NMR spectroscopy.

A brownish wax having the following structure is obtained:

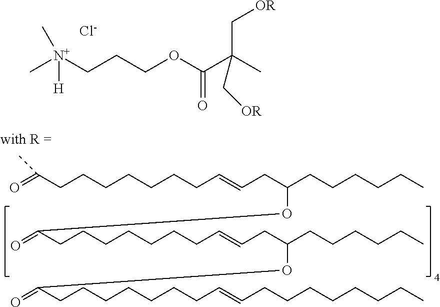

### Example 5 (not According to the Invention)

### Synthesis of a Tertiary Amino Alcohol Ester of the [(Ricinoleic Acid)₂-Oleic Acid] Estolide

The protocol outlined for example 1 was repeated with the exception that the n-heptane is not removed under reduced pressure.

Instead, the n-heptane solution was transferred to a separation funnel and mixed with a basic mixture containing 100 g deionized water, 30 g NaCl and 10 g NaOH. After phase separation, the aqueous (bottom) phase was removed. A mixture containing 100 g deionized water and 30 g NaCl was added to the upper organic phase. After mixing and phase separation, the bottom deionized water/NaCl phase was removed. This process using deionized water/NaCl mixtures was repeated 5 times. Finally, the organic phase was dried three times with 30 g NaCl. The n-heptane was removed under reduced pressure (30° C./2 h/20 mmHg). The formation of the ester of the tertiary aminoalcohol was confirmed by means of ¹H NMR spectroscopy.

A low viscous brownish liquid having the following structure was obtained:

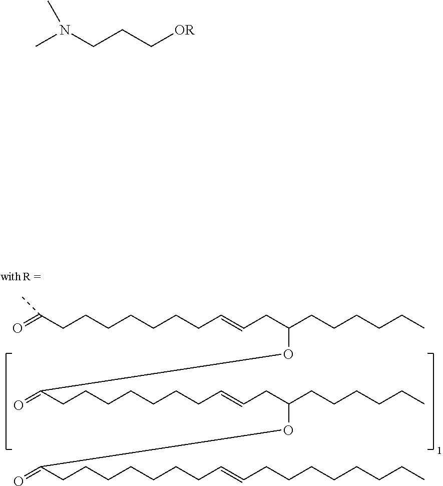

### Example 6

### Synthesis of a Glycerol Diglycidyl Ether Based Quat Using the Amine Salt of a Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.5 g (0.01093 mol epoxy groups; specific epoxy content 0.00729 mol epoxy groups/1 g) glycerol diglycidyl ether, 10.55 g (0.01093 mol) of the amine salt from example 1, 55 g 2-propanol and 0.14 g deionized water were mixed at room temperature. The mixture was heated to 80° C. for 11 h. Afterwards, volatiles were removed under reduced pressure (40° C./2 h/20 mmHg). The complete conversion of the epoxy groups was confirmed by means of ¹H NMR spectroscopy.

A brownish viscous oil having the following structure was obtained:

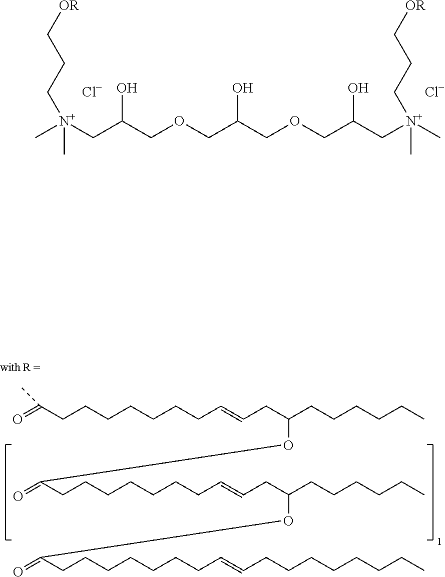

### Example 7

### Synthesis of a Glycerol Triglycidyl Ether Based Quat Using the Amine Salt of a Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.5 g (0.01004 mol epoxy groups; specific epoxy content 0.00669 mol epoxy groups/1 g) glycerol triglycidyl ether, 10.14 g (0.01004 mol) of the amine salt from example 1, 50 g 2-propanol and 0.15 g deionized water were mixed at room temperature. The mixture was heated to 80° C. for 16 h. Afterwards, volatiles were removed under reduced pressure (40° C./2 h/20 mmHg). The complete conversion of the epoxy groups was confirmed by means of ¹H NMR spectroscopy.

A brownish viscous oil having the following structure was obtained:

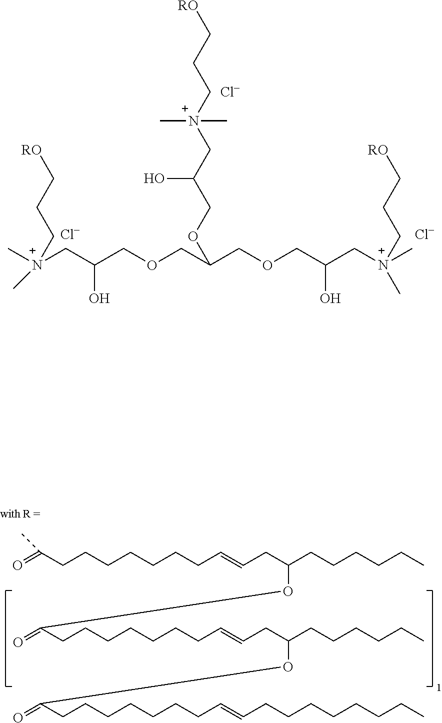

### Example 8

### Synthesis of a Diglycerol Triglycidyl Ether Based Quat Using the Amine Salt of a Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.62 g (0.01004 mol epoxy groups; specific epoxy content 0.00621 mol epoxy groups/1 g) diglycerol triglycidyl ether, 10.14 g (0.01004 mol) of the amine salt from example 1, 50 g 2-propanol and 0, 15 g deionized water were mixed at room temperature. The mixture was heated to 80° C. for 14 h. Afterwards, volatiles were removed under reduced pressure (40° C./2 h/20 mmHg). The complete conversion of the epoxy groups was confirmed by means of ¹H NMR spectroscopy.

A brownish viscous oil having the following structure was obtained:

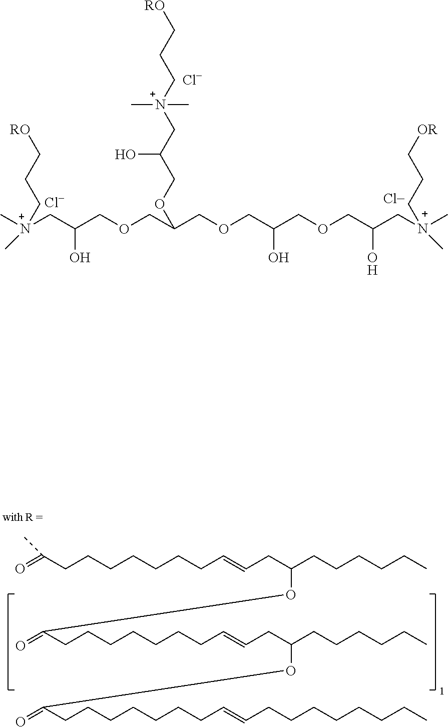

### Example 9

### Synthesis of a Polyglycerol Polyglycidyl Ether Based Quat Using the Amine Salt of a Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.68 g (0.01004 mol epoxy groups; specific epoxy content 0.00597 mol epoxy groups/1 g) polyglycerol polyglycidyl ether, 10.14 g (0.01004 mol) of the amine salt from example 1, 50 g 2-propanol and 0.15 g deionized water were mixed at room temperature. The mixture was heated to 80° C. for 12 h. Afterwards, volatiles were removed under reduced pressure (40° C./2 h/20 mmHg). The complete conversion of the epoxy groups was confirmed by means of ¹H NMR spectroscopy.

A brownish viscous oil having the following approximate structure was obtained:

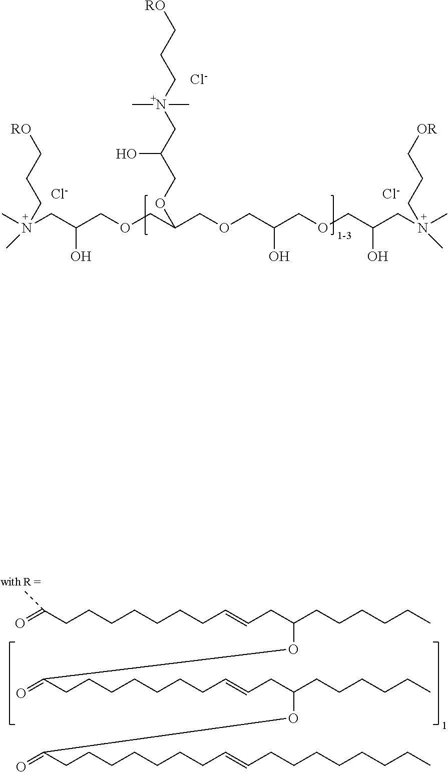

### Example 10

### Synthesis of a Glycerol Diglycidyl Ether Based Quat Using the Amine Salt of a Tertiary Amino Alcohol Ester of the [(Ricinoleic Acid)s-Oleic Acid] Estolide

In a 100 ml three-necked bottle, equipped with refluxing condenser, thermometer and mechanic stirrer 0.69 g (0.0045 mol epoxy groups; specific epoxy content 0.00729 mol epoxy groups/1 g) glycerol diglycidyl ether, 8.42 g (0.0045 mol) of the amine salt from example 2, 50 g 2-propanol and 0.1 g deionized water were mixed at room temperature. The mixture was heated to 80° C. for 14 h. Afterwards, volatiles were removed under reduced pressure (40° C./2 h/20 mmHg). The complete conversion of the epoxy groups was confirmed by means of ¹H NMR spectroscopy.

A brownish viscous oil having the following structure was obtained:

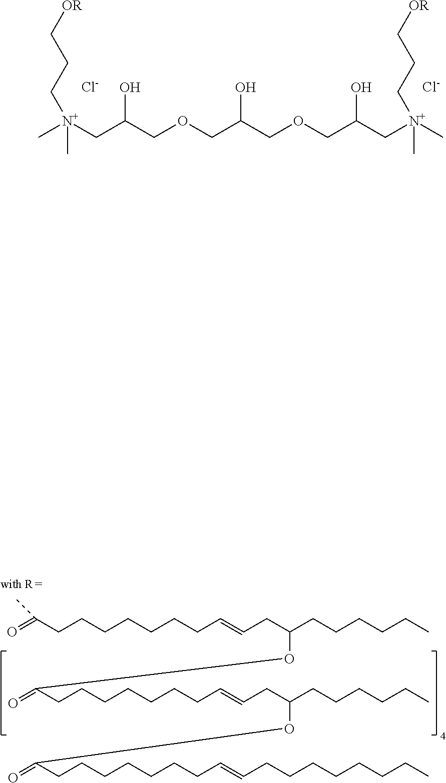

### Example 11

### Synthesis of a Glycerol Diglycidyl Ether Based Quat Using the Amine Salt of a Tertiary Amino Alcohol Ester of the Alpha Branched Bis-[(Ricinoleic Acid)₂-Oleic Acid] Estolide

In a 100 ml three-necked bottle, equipped with refluxing condenser, thermometer and mechanic stirrer 0.46 g (0.0031 mol epoxy groups; specific epoxy content 0.00729 mol epoxy groups/1 g) glycerol diglycidyl ether, 6.0 g (0.0031 mol) of the amine salt from example 3, 50 g 2-propanol and 0.1 g deionized water were mixed at room temperature. The mixture was heated to 80° C. for 11 h. Afterwards, volatiles were removed under reduced pressure (40° C./2 h/20 mmHg). The complete conversion of the epoxy groups was confirmed by means of ¹H NMR spectroscopy. A brownish wax having the following approximate structure was obtained:

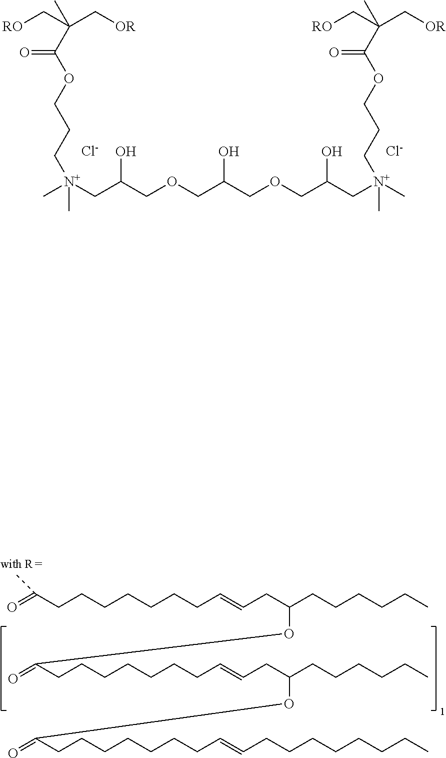

### Example 11a

### Synthesis of a Glycerol Diglycidyl Ether Based Quat Using the Amine Salt of a Tertiary Amino Alcohol Ester of the Alpha Branched Bis-[(Ricinoleic Acid)₂- Stearic Acid] Estolide

In a 250 ml three-necked bottle, equipped with refluxing condenser, thermometer and mechanic stirrer 2.98 g (0.02417 mol epoxy groups; specific epoxy content 0.00729 mol epoxy groups/1 g) glycerol diglycidyl ether, 45.3 g (0.02417 mol) of the amine salt from example 3b, 200 g 2-propanol and 0.15 g deionized water were mixed at room temperature. The mixture was heated to 80° C. for 9 h. Afterwards, volatiles were removed under reduced pressure (40° C./2 h/20 mmHg). The complete conversion of the epoxy groups was confirmed by means of ¹H NMR spectroscopy.

A brownish wax having the following structure was obtained:

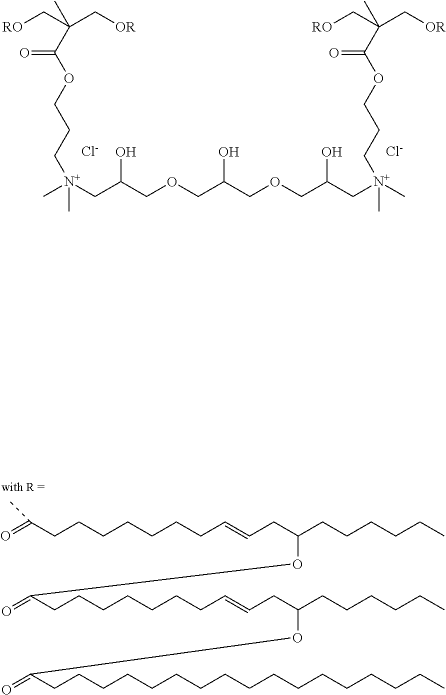

### Example 11b

### Synthesis of a Glycerol Diglycidyl Ether Based Quat Using the Amine Salt of a Tertiary Amino Alcohol Ester of the Alpha Branched Bis-(Ricinoleic Acid-12 Hydroxy Stearic Acid-Oleic Acid) Estolide

In a 250 ml three-necked bottle, equipped with refluxing condenser, thermometer and mechanic stirrer 3.13 g (0.0228 mol epoxy groups; specific epoxy content 0.00729 mol epoxy groups/1 g) glycerol diglycidyl ether, 44.1 g (0.0228 mol) of the amine salt from example 3c, 200 g 2-propanol and 0.15 g deionized water were mixed at room temperature. The mixture was heated to 80° C. for 9 h. Afterwards, volatiles were removed under reduced pressure (40° C./2 h/20 mmHg). The complete conversion of the epoxy groups was confirmed by means of ¹H NMR spectroscopy.

A brownish wax having the following approximate structure was obtained:

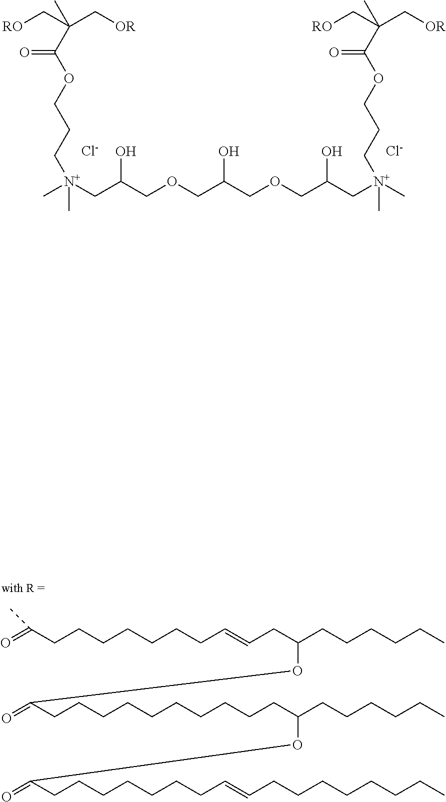

### Example 11c

### Synthesis of a Glycerol Diglycidyl Ether Based Quat Using the Amine Salt of a Tertiary Amino Alcohol Ester of the Alpha Branched Bis-(12 Hydroxy Stearic Acid-Ricinoleic Acid-Oleic Acid) Estolide

In a 250 ml three-necked bottle, equipped with refluxing condenser, thermometer and mechanic stirrer 3.33 g (0.0243 mol epoxy groups; specific epoxy content 0.00729 mol epoxy groups/1 g) glycerol diglycidyl ether, 45.6 g (0.0243 mol) of the amine salt from example 3d, 200 g 2-propanol and 0.15 g deionized water were mixed at room temperature. The mixture was heated to 80° C. for 9 h. Afterwards, volatiles were removed under reduced pressure (40° C./2 h/20 mmHg). The complete conversion of the epoxy groups was confirmed by means of ¹H NMR spectroscopy.

A brownish wax having the following approximate structure was obtained:

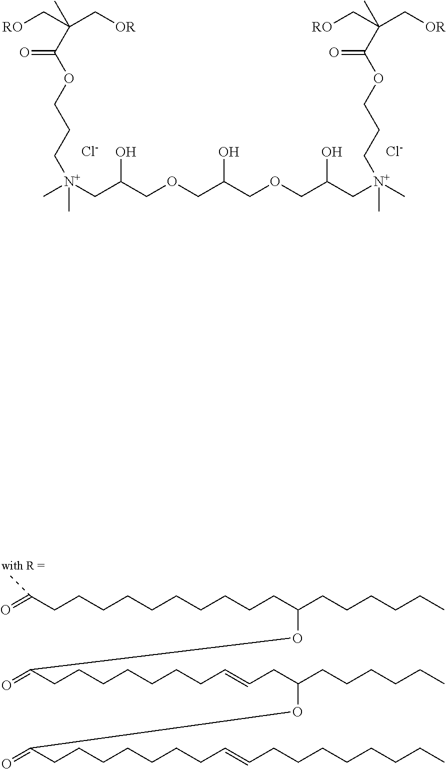

### Example 12

### Synthesis of a Glycerol Diglycidyl Ether Based Bis Quat Using the Amine Salt of a Tertiary Amino Alcohol Ester of the Alpha Branched Bis-[(Ricinoleic Acid)s-Oleic Acid] Estolide

In a 100 ml three-necked bottle, equipped with refluxing condenser, thermometer and mechanic stirrer 0.286 g (0.00209 mol epoxy groups; specific epoxy content 0.00729 mol epoxy groups/1 g) glycerol diglycidyl ether, 7.5 g (0.00209 mol) of the amine salt from example 4, 50 g 2-propanol and 0.1 g deionized water were mixed at room temperature. The mixture was heated to 80° C. for 11 h. Afterwards, volatiles were removed under reduced pressure (40° C./2 h/20 mmHg). The complete conversion of the epoxy groups was confirmed by means of ¹H NMR spectroscopy.

A brownish wax having the following approximate structure was obtained:

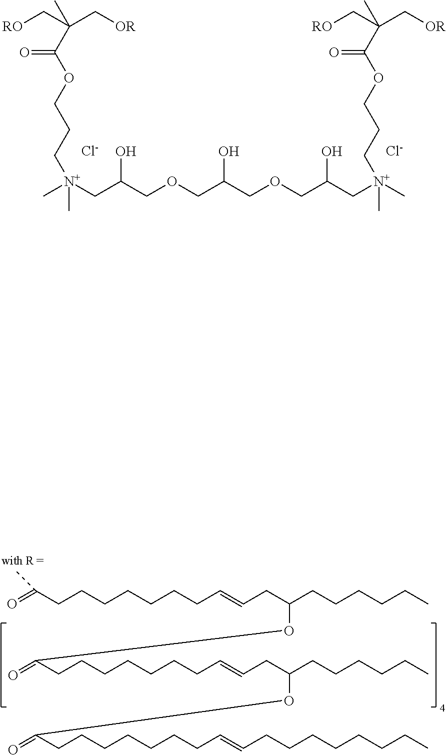

### Example 13

Synthesis of a Glycerol Diglycidyl Ether Based Quat Using the Tertiary Amino Alcohol Ester of the [(Ricinoleic Acid)₂-Oleic Acid] Estolide with Same Estolid Counterion

In a 100 ml three-necked bottle, equipped with refluxing condenser, thermometer and mechanic stirrer, 1.0 g (0.00729 mol epoxy groups; specific epoxy content 0.00729 mol epoxy groups/1 g) glycerol diglycidyl ether, 6.77 g (0.00729 mol) of the tertiary amine from example 5, 6.15 g (0.00729 mol) of the [(ricinoleic acid)₂-oleic acid] from synthesis example 2, 50 g 2-propanol and 0.15 g deionized water were mixed at room temperature. The mixture was heated to 80° C. for 11 h. Afterwards, volatiles were removed under reduced pressure (40° C./2 h/20 mmHg). The complete conversion of the epoxy groups was confirmed by means of ¹H NMR spectroscopy.

A brownish viscous oil having the following approximate structure was obtained:

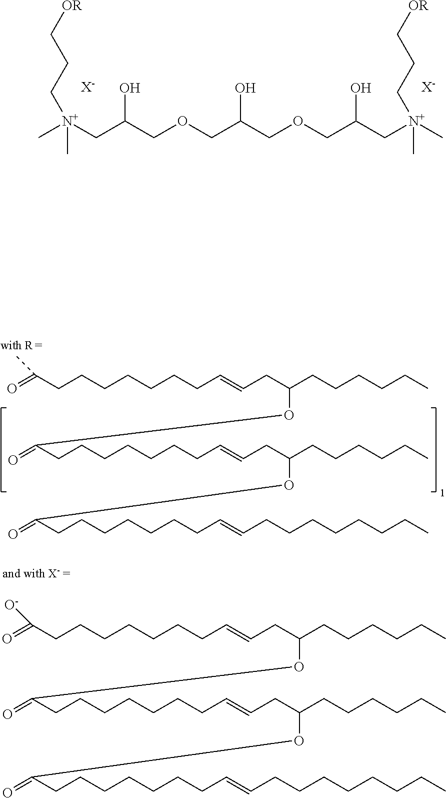

### Example 14

Synthesis of a Glycerol Triglycidyl Ether Based Ter(Quat) Using the Tertiary Amino Alcohol Ester of the [(Ricinoleic Acid)₂-Oleic Acid] Estolide with Same Estolide as Counterion.

In a 100 ml three-necked bottle, equipped with refluxing condenser, thermometer and mechanic stirrer 1.0 g (0.00669 mol epoxy groups; specific epoxy content 0.00669 mol epoxy groups/1 g) glycerol triglycidyl ether, 6.21 g (0.00669 mol) of the tertiary amine from example 5, 5.64 g (0.00669 mol) of the [(ricinoleic acid)₂-oleic acid] from synthesis example 2, 50 g 2-propanol and 0.15 g deionized water were mixed at room temperature. The mixture was heated to 80° C. for 16 h. Afterwards, volatiles were removed under reduced pressure (40° C./1 h/20 mmHg). The complete conversion of the epoxy groups was confirmed by means of ¹H NMR spectroscopy.

A brownish viscous oil having the following structure was obtained:

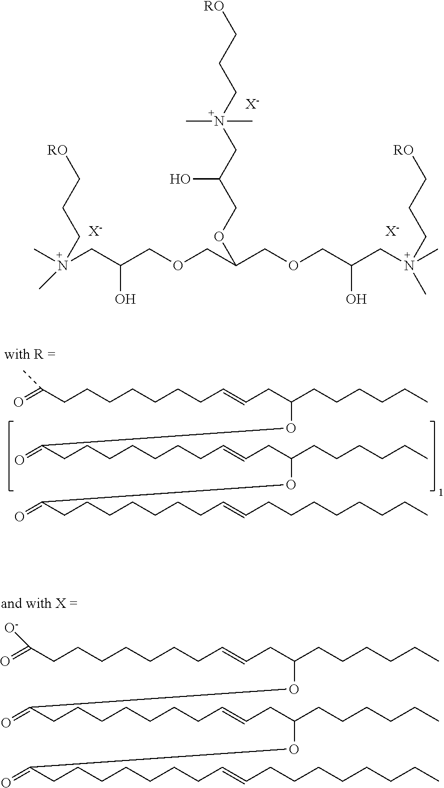

### Example 15

### Synthesis of a Diglycerol Triglycidyl Ether Based Ter(Quat) Using the Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.08 g (0.00669 mol epoxy groups; specific epoxy content 0.00621 mol epoxy groups/1 g) diglycerol triglycidyl ether, 6.21 g (0.00669 mol) of the tertiary amine from example 5, 5.64 g (0.00669 mol) of the [(ricinoleic acid)₂-oleic acid] from synthesis example 2, 50 g 2-propanol and 0.15 g deionized water were mixed at room temperature. The mixture was heated to 80° C. for 14 h. Afterwards, volatiles were removed under reduced pressure (40° C./1 h/20 mmHg). The complete conversion of the epoxy groups was confirmed by means of ¹H NMR spectroscopy.

A brownish viscous oil having the following structure was obtained:

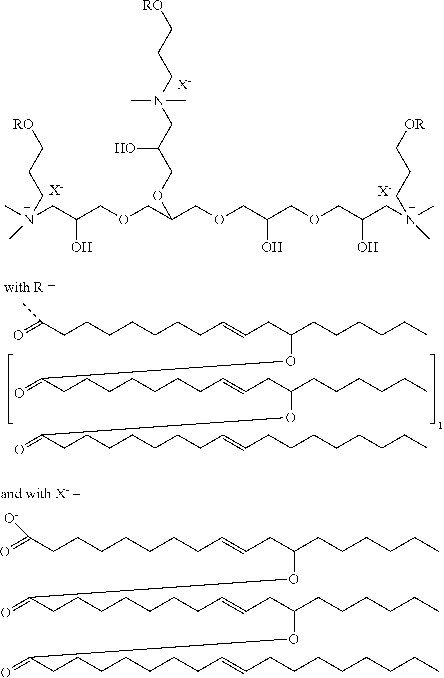

### Example 16

### Synthesis of a Polyglycerol Polyglycidyl Ether Based Quat Using the Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.12 g (0.00669 mol epoxy groups; specific epoxy content 0.00597 mol epoxy groups/1 g) polyglycerol polyglycidyl ether, 6.21 g (0.00669 mol) of the tertiary amine from example 5, 5.64 g (0.00669 mol) of the [(ricinoleic acid)₂-oleic acid] from synthesis example 2, 50 g 2-propanol and 0.15 g deionized water were mixed at room temperature. The mixture was heated to 80° C. for 13 h. Afterwards, volatiles were removed under reduced pressure (40° C./1 h/20 mmHg). The complete conversion of the epoxy groups was confirmed by means of ¹H NMR spectroscopy.

A brownish viscous oil having the following approximate structure was obtained:

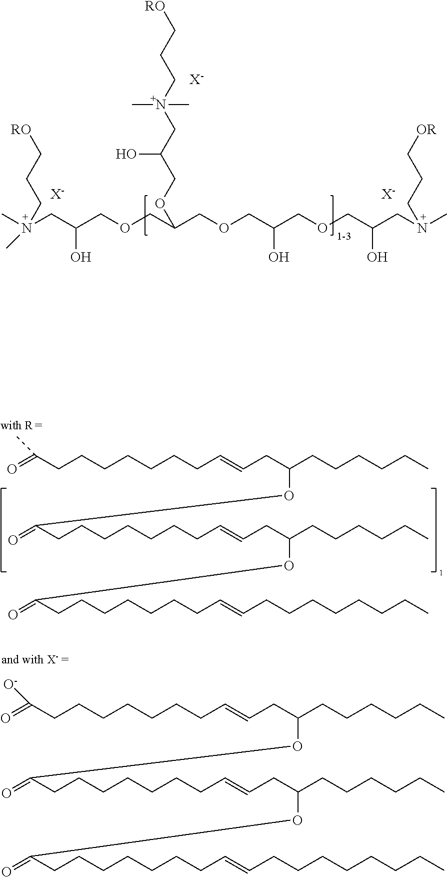

### Example 17

Synthesis of a Diethylene Glycol Based Quat Using the Tertiary Amino Alcohol Ester of the [(Ricinoleic Acid)₂-Oleic Acid] Estolide from Example 5

In a 100 ml three-necked bottle, equipped with refluxing condenser, thermometer and mechanic stirrer 9.285 g (0.01 mol) of the tertiary amine from example 5, 1.295 g (0.01 mol CH₂Cl) of the bis-(chloro acetic acid) ester of diethylene glycol and 40 g 2-propanol were mixed at room temperature. The mixture was heated to 80° C. for 12 h. Afterwards, volatiles were removed under reduced pressure (40° C./2 h/20 mmHg). The complete conversion of the CH₂Cl groups was confirmed by means of ¹H NMR spectroscopy.

A brownish viscous wax havinq the followinq approximate structure was obtained:

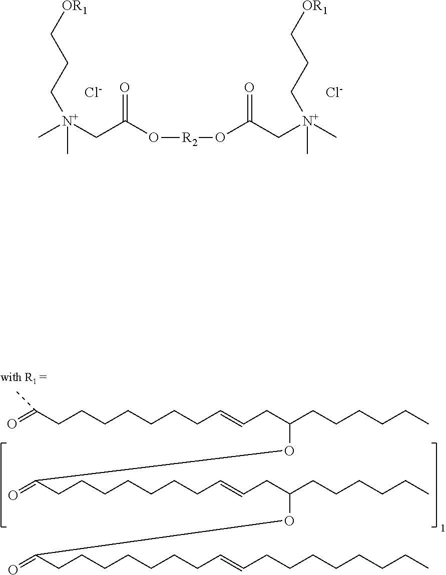

and with R₂=—CH₂CH₂OCH₂CH₂—.

### Example 18

### Synthesis of a 1,4-Butanediol Based Quat Using the Amine Salt of a Tertiary Amino Alcohol Ester of the [(Ricinoleic Acid)₂-Oleic Acid] Estolide of Example 1.

In a 100 ml three-necked bottle, equipped with refluxing condenser, thermometer and mechanic stirrer, 0.6 g (0.00593 mol epoxy groups) 1,4 butanediol diglycidyl ether, 5.72 g (0.00593 mol) of the amine salt from example 1, 55 g 2-propanol and 0.1 g deionized water were mixed at room temperature. The mixture was heated to 80° C. for 11 h. Afterwards, volatiles were removed under reduced pressure (40° C./2 h/20 mmHg). The complete conversion of the epoxy groups was confirmed by means of ¹H NMR spectroscopy.

A brownish oily liquid having the following approximate structure was obtained:

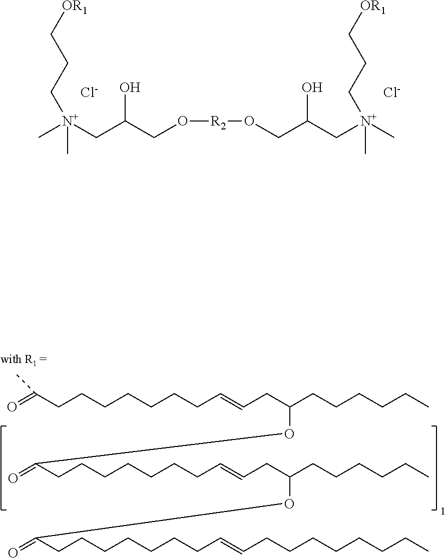

and with R₂=—CH₂CH₂CH₂CH₂—

### Example 19

### Synthesis of a 1,4-Butanediol-Succinic Acid Ester Based Quat Using the Amine Salt of a Tertiary Amino Alcohol Ester of the [(Ricinoleic Acid)₂-Oleic Acid] Estolide

In a 100 ml three-necked bottle, equipped with refluxing condenser, thermometer and mechanic stirrer 1.2 g (0.01186 mol epoxy groups) 1,4 butanediol diglycidyl ether, 0.35 g (0.00593 mol COOH) succinic acid, 55 g 2-propanol and 0.03 g trimethylamine were mixed at room temperature (for the formation of R₂). The mixture was heated to 80° C. for 15 h. The partial conversion of the epoxy groups was confirmed by means of ¹H NMR spectroscopy. Afterwards, 5.72 g (0.00593 mol) of the amine salt from example 1 were added and the reaction continued at 80° C. for 10 h. Volatiles were removed under reduced pressure (40° C./2 h/20 mmHg). The complete conversion of the epoxy groups was confirmed by means of ¹H NMR spectroscopy.

A brownish high viscous liquid having the following structure was obtained:

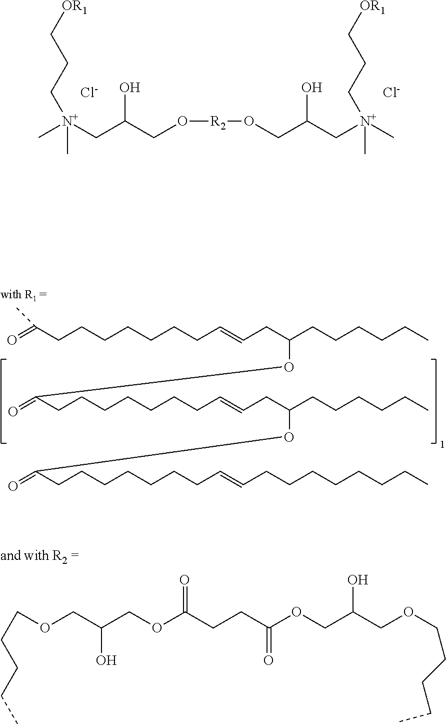

### Example 20

### Synthesis of a Glycerol Estolide Based Di-Quat

In a 100 ml three-necked bottle, equipped with refluxing condenser, thermometer and mechanic stirrer, 25 g (0.0198 mol CH₂Cl groups) of the chloro acetic acid ester derivative of a glycerol based estolide from synthesis example 6, 1.71 g (0.0099 mol) N,N,N′,N′ tetramethyl-1,6-hexanediamine and 50 g 2-propanol were mixed at room temperature. The mixture was heated to 80° C. for 7 h. Volatiles were removed under reduced pressure (40° C./2 h/20 mmHg). The complete conversion of the CH₂Cl groups was confirmed by means of ¹H NMR spectroscopy.

A brownish oily liquid having the following approximate structure was obtained:

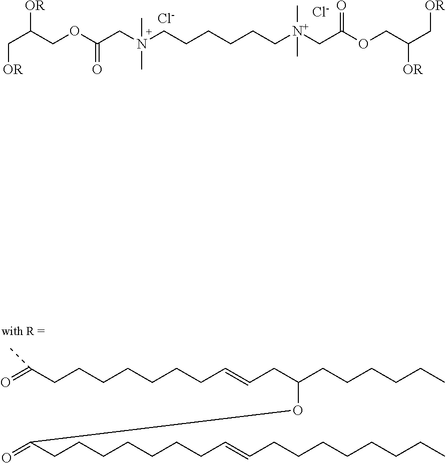

### Example 21

### Synthesis of a Castor Oil Estolide Based Di-Quat N-Terminated by Castor Oil Estolide

In a 100 ml three-necked bottle, equipped with refluxing condenser, thermometer and mechanic stirrer 25 g (0.0119 mol CH₂Cl groups) of the chloro acetic acid ester derivative of a castor oil based estolide from synthesis example 7, 1.03 g (0.006 mol) N,N,N′,N′ tetramethyl-1,6-hexanediamine and 60 g 2-propanol were mixed at room temperature. The mixture was heated to 80° C. for 15 h. Volatiles were removed under reduced pressure (40° C./2 h/20 mmHg). The complete conversion of the CH₂Cl groups was confirmed by means of ¹H NMR spectroscopy.

A brownish oily liquid having the following structure was obtained:

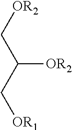

with R₁ (linking to two of the above R₂ containing elements)=

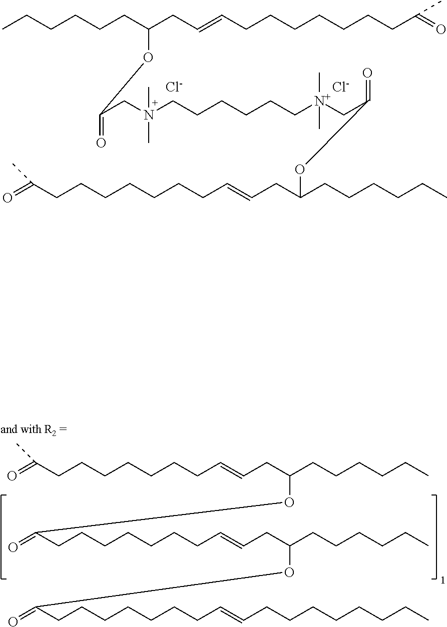

### Example 22

### Synthesis of a Branched Castor Oil Estolide Based Quat N-Terminated by Castor Oil Estolide

In a 100 ml three-necked bottle, equipped with refluxing condenser, thermometer and mechanic stirrer 0.22 g (0.0016 mol epoxy groups; specific epoxy content 0.00729 mol epoxy groups/1 g) glycerol diglycidyl ether, 0.30 g (0.0016 mol NH groups) (CH₃)₂NCH₂CH₂CH₂NHCH₂CH₂CH₂N(CH₃)₂ and 55 g 2-propanol were mixed at room temperature. The mixture was heated to 80° C. for 8 h. The complete conversion of the epoxy groups was confirmed by means of ¹H NMR spectroscopy.

6.73 g (0.0032 mol CH₂Cl groups) of the chloro acetic acid ester derivative of a castor oil based estolide from synthesis example 7 were added and reaction continued at 80° C. for 10 h. Volatiles were removed under reduced pressure (40° C./2 h/20 mmHg).

The conversion of the CH₂Cl groups as determined by means of ¹H NMR spectroscopy was 97%.

A brownish oily liquid having the following approximate structure was obtained:

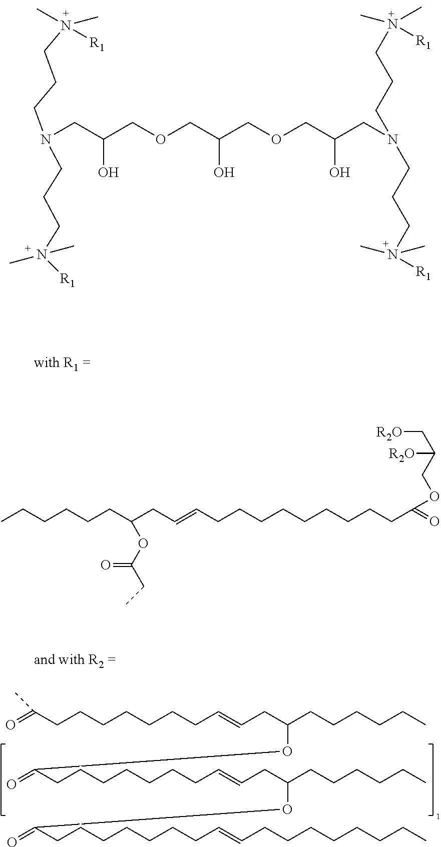

### Example 23

Synthesis of a N⁺,N⁺-Saccharide Modified Castor Oil Estolide Based Di Quat

In a 100 ml three-necked bottle, equipped with refluxing condenser, thermometer and mechanic stirrer 0.44 g (0.0032 mol epoxy groups; specific epoxy content 0.00729 mol epoxy groups/1 g) glycerol diglycidyl ether, 0.62 g (0.0032 mol) N-methylglucamine and 66 g 1,3-propanediol are mixed at room temperature. The mixture was heated to 800° C. for 8.5 h. The complete conversion of the epoxy groups was confirmed by means of ¹H NMR spectroscopy.

6.73 g (0.0032 mol CH₂Cl groups) of the chloro acetic acid ester derivative of a castor oil based estolide from synthesis example 7 was added and reaction continued at 80° C. for 13 h. The conversion of the CH₂Cl groups was confirmed by means of ¹H NMR spectroscopy.

A yellowish oily liquid containing a quat having the following approximate structure was obtained:

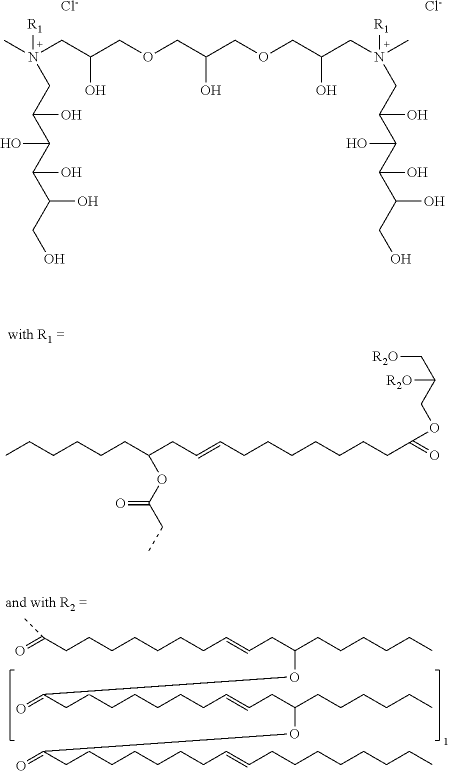

### Example 24

### Synthesis of a Castor Oil Estolide Based Derivative Having Pending Quat Moieties

In a 100 ml three-necked bottle, equipped with refluxing condenser, thermometer and mechanic stirrer 0.44 g (0.0032 mol epoxy groups; specific epoxy content 0.00729 mol epoxy groups/1 g) glycerol diglycidyl ether, 0.163 g (0.0016 mol) N,N-dimethyl-1,3-propanediamine and 55 g 2-propanol are mixed at room temperature. The mixture was heated to 80° C. for 8.5 h. The complete conversion of the epoxy groups was confirmed by means of ¹H NMR spectroscopy. 3.36 g (0.0016 mol CH₂Cl groups) of the chloro acetic acid ester derivative of a castor oil based estolide from synthesis example 7 was added and the reaction was continued at 80° C. for 13 h. Volatiles were removed under reduced pressure (40° C./2 h/20 mmHg). The conversion of the CH₂Cl groups was confirmed by means of ¹H NMR spectroscopy.

A yellowish oily liquid containing a quat having the following approximate structure was obtained:

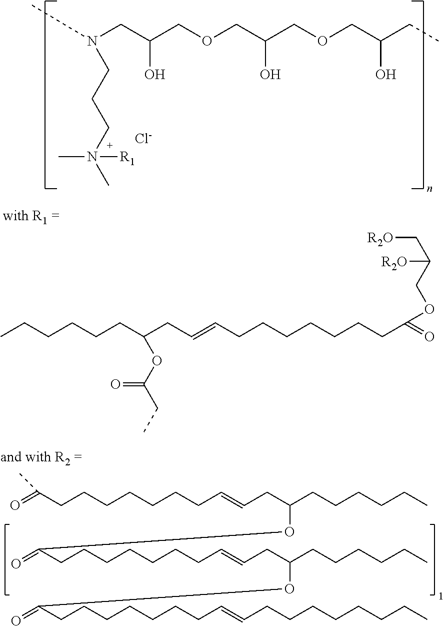

The number of repeating units n in the above-shown structural formula of the compound obtained by the above procedure is within the range of n≤30.

### Example 25

### Synthesis of a Polyglycerol-Polyricinolate (PGPR) Based Quat Derivative

In a 100 ml three-necked bottle, equipped with refluxing condenser, thermometer and mechanic stirrer, dropping funnel and gas outlet tube, 60 g (0.0964 mol OH groups; specific OH content 0.00161 mol OH groups/1 g material) of a commercial polyglycerol-polyricinolate were placed at 31° C. Upon stirring, 8.45 g (0.0748 mol) chloro acetic acid chloride were added over 30 minutes. The temperature increased to 39° C. The mixture was heated to 70° C. and the temperature was maintained for 3 h. Afterwards, volatiles were removed under reduced pressure (40° C./1 h/20 mmHg). The formation of the chloro acetic acid ester structures was confirmed by means of ¹H NMR spectroscopy.

In a 100 ml three-necked bottle, equipped with refluxing condenser, thermometer and mechanic stirrer, 27 g (0.0307 mol CH₂Cl) of the above PGPR-chloro acetic acid ester derivative, 2.64 g (0.0153 mol) N,N,N′,N′ tetramethyl-1,6-hexanediamine and 40 g 2-propanol were mixed at room temperature and heated to 80° C. for 6 h. Volatiles were removed under reduced pressure (40° C./2 h/20 mmHg). The conversion of the CH₂Cl groups was confirmed by means of ¹H NMR spectroscopy.

A yellowish wax was obtained.

### Example 26

### Synthesis of a Polyglycerol-Polyricinolate (PGPR) Based Quat Derivative

In a 100 ml three-necked bottle, equipped with refluxing condenser, thermometer and mechanic stirrer, dropping funnel and gas outlet tube 60 g (0.0964 mol OH groups; specific OH content 0.00161 mol OH groups/1 g material) of a commercial polyglycerol-polyricinolate were placed at 24° C. Upon stirring, 4.83 g (0.0427 mol) chloro acetic acid chloride were added over 30 minutes. The temperature increased to 28° C. The mixture was heated to 70° C. and the temperature maintained for 3 h. Afterwards, volatiles were removed under reduced pressure (40° C./1.5 h/20 mmHg). The formation of the chloro acetic acid ester structures was confirmed by means of ¹H NMR spectroscopy.

In a 100 ml three-necked bottle, equipped with refluxing condenser, thermometer and mechanic stirrer, 27 g (0.0193 mol CH₂Cl) of the above PGPR-chloro acetic acid ester derivative, 1.66 g (0.00962 mol) N,N,N′,N′ tetramethyl-1,6-hexanediamine and 40 g 2-propanol were mixed at room temperature and heated to 80° C. for 8 h. Volatiles are removed under reduced pressure (40° C./1 h/20 mmHg). The conversion of the CH₂Cl groups was confirmed by means of ¹H NMR spectroscopy.

A brownish wax was obtained.

### Example 27

### Synthesis of a Polyquat Having Estolide Moieties Attached to Glycerol Units

In a 100 ml three-necked bottle, equipped with refluxing condenser, thermometer and mechanic stirrer, 2.71 g (0.0197 mol epoxy groups; specific epoxy content 0.00729 mol epoxy groups/1 g) glycerol diglycidyl ether, 1.87 g (0.0197 mol) chloro acetic acid, 0.2 g triethylamine and 63.4 g methoxy propyl acetate were mixed at room temperature. The mixture was heated to 100° C. for 8 h. The complete conversion of the epoxy groups was confirmed by means of ¹H NMR spectroscopy.

The mixture was cooled to room temperature and 11.48 g (0.0197 mol) of the [(ricinoleic acid)₁-oleic acid] chloride used as intermediate in synthesis example 3 were added over 15 minutes. The temperature was increased to 100° C. and was maintained for 7 h. The conversion of the C(O)CL groups was confirmed by means of ¹H NMR spectroscopy.

3.4 g (0.0197 mol) N,N,N′,N′ tetramethyl-1,6-hexanediamine were added and the reaction was continued at 100° C. for 7 h. The conversion of the CH₂Cl groups was confirmed by means of ¹H NMR spectroscopy. Volatiles were removed under reduced pressure (80° C./5 h/20 mmHg).

A brownish wax having the following approximate structure was obtained:

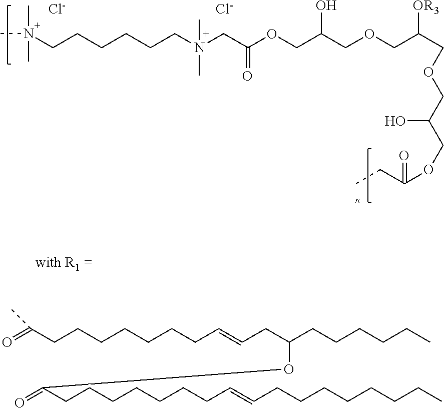

The number of repeating units n in the above-shown structural formula of the compound obtained by the above procedure is within the range of n≤30.

### Example 28

### Synthesis of a Di-Quat Having Estolide Moieties Attached to Glycerol Units Bound to Each N⁺ Group

In a 100 ml three-necked bottle, equipped with refluxing condenser, thermometer and mechanic stirrer, 10.5 g (0.0765 mol epoxy groups; specific epoxy content 0.00729 mol epoxy groups/1 g) glycerol diglycidyl ether, 43.09 g g (0.0765 mol) of the (ricinoleic acid-oleic acid) from synthesis example 1, 0.56 g (0.0066 mol) N,N,N′,N′ tetramethyl-1,6-hexanediamine and 24.3 g propylene glycol monomethyl ether were mixed at room temperature. The mixture was heated to 100° C. for 15 h. The complete conversion of the epoxy groups was confirmed by means of ¹H NMR spectroscopy.

The solvent was removed under reduced pressure (70° C./4 h/20 mmHg). Afterwards, 2.67 g (0.0236 mol) chloro acetic acid chloride were added. The temperature increased to 48° C. Volatiles were removed under reduced pressure (40° C./1 h/20 mmHg). 2.03 g (0.0118 mol) N,N,N′,N′ tetramethyl-1,6-hexanediamine and 24.3 g propylene glycol monomethyl ether were added and the mixture was heated to 100° C. for 13 h.

Volatiles were removed under reduced pressure (80° C./5 h/20 mmHg). The conversion of the CH₂Cl groups was confirmed by means of ¹H NMR spectroscopy.

A brownish wax having the following approximate structure was obtained:

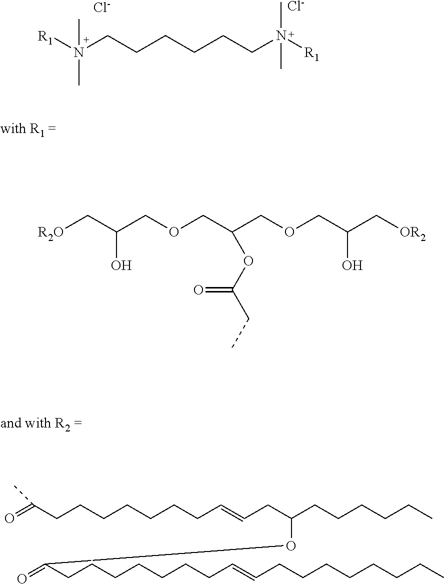

### Synthesis Example 29a

Synthesis of a [(Ricinoleic Acid)₆-Succinic Acid-(Ricinoleic Acid)₆] Diacid Estolide

Two 250 ml three-necked bottles A and B, equipped with refluxing condenser, thermometer and magnetic stirrer, dropping funnel and gas outlet tube were flushed with nitrogen.

Bottle A was used to react the starting material dicarboxylic acid chloride succinyl dichloride or fatty acid chlorides with ricinoleic acid yielding a chain extended fatty ester acid. Subsequent addition of SOCl₂ yielded the corresponding fatty ester acid chloride.

Bottle B was used to react the formed fatty ester acid chloride with ricinoleic acid, yielding a chain extended fatty ester acid. Subsequent addition of SOCl₂ yielded the corresponding fatty ester acid chloride. This fatty acid chloride was transferred back to bottle A and was reacted with fresh ricinoleic acid. The above-described cycle was repeated until the estolide [(ricinoleic acid)₆-succinic acid-(ricinoleic acid)₆] was prepared.

General procedure for the synthesis of chain extended fatty ester acids: The calculated amount of ricinoleic acid was placed in a bottle. An equimolar amount of fatty ester acid chloride was added slowly at room temperature. In order to complete the reaction, the temperature was increased to 80° C. for 3 h. The complete conversion of the OH groups was determined by means of ¹H NMR spectroscopy.

General procedure for the synthesis of fatty ester acid chlorides: The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)CL groups was determined by means of ¹H NMR spectroscopy.

The following table summarizes the materials and the quantities used.

| | | Dicarboxylic | | | |
|---|---|---|---|---|---|
| | | acid | | | |
| | | chloride | | | |
| | | starting | | | |
| | Dicarboxylic acid | material or | | | |
| | chloride starting | fatty ester | | SOCl₂ | |
| | material or fatty | derivative | ricinoleic acid | amount | |
| bottle | ester derivative | amount [g] | amount [g] | [g] | target product |
| A | succinyl dichloride | 30.81 | 118.68 | | [(rici)₁-succ-(rici)₁] diacid |
| A | [(rici)₁-succ-(rici)₁] | 135 | | 73.1 | [(rici)₁-succ-(rici)₁] |
| | diacid | | | | dichloride |
| B | [(rici)₁-succ-(rici)₁] | 72.17 | 60.18 | | [(rici)₂-succ-(rici)₂] diacid |
| | dichloride | | | | |
| B | [(rici)₂-succ-(rici)₂] | 125 | | 40.32 | [(rici)₂-succ-(rici)₂] |
| | diacid | | | | dichloride |
| A | [(rici)₂-succ-(rici)₂] | 88.63 | 41.43 | | [(rici)₃-succ-(rici)₃] diacid |
| | dichloride | | | | |
| A | [(rici)₃-succ-(rici)₃] | 125 | | 39.62 | [(rici)₃-succ-(rici)₃] |
| | diacid | | | | dichloride |
| B | [(rici)₃-succ-(rici)₃] | 97.27 | 31.59 | | [(rici)₄-succ-(rici)₄] diacid |
| | dichloride | | | | |
| B | [(rici)₄-succ-(rici)₄] | 125 | | 24.71 | [(rici)₄-succ-(rici)₄] |
| | diacid | | | | dichloride |
| A | [(rici)₄-succ-(rici)₄] | 102.59 | 25.53 | | [(rici)₅-succ-(rici)₅] diacid |
| | dichloride | | | | |
| A | [(rici)₅-succ-(rici)₅] | 125 | | 22.37 | [(rici)₅-succ-(rici)₅] |
| | diacid | | | | dichloride |
| B | [(rici)₅-succ-(rici)₅] | 106.20 | 21.42 | | [(rici)₆-succ-(rici)₆] diacid |
| | dichloride | | | | |

Note:
The term “rici” replaces the term “ricinoleic acid” in denoting a ricinoleyl radical, the term “succ” replaces the term “succinic acid” in denoting a succinyl radical.

A brownish, transparent oil essentially having the following structure [(rici)₆-succinyl-(rici)₆] was obtained:

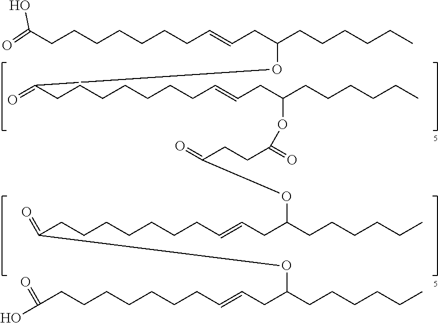

### Example 29b

Synthesis of a Di-Quat Based on a Tertiary Amino Alcohol Ester of the [(Ricinoleic Acid)₆-Succinic Acid-(Ricinoleic Acid)₆] Diacid Estolide

In a 250 ml three-necked bottle, equipped with refluxing condenser, thermometer, magnetic stirrer, dropping funnel and gas outlet tube, 59.57 g (0.0171 mol) of the [(ricinoleic acid)₆-succinic acid-(ricinoleic acid)₆] diacid estolide of synthesis example 29a are mixed at 60° C. with 15.64 g (0.131 mol) SOCl₂. The mixture was heated to 80° C. for 3.5 h. Afterwards, the excess of SOCl₂ was removed under reduced pressure (80° C./1 h/20 mmHg). The formation of the corresponding acid chloride was confirmed by means of ¹H NMR spectroscopy.

After cooling to room temperature, 140 g n-heptane and 3.52 (0.0342 mol) of (CH₃)₂NCH₂CH₂CH₂OH were added over 5 minutes. The temperature increased from 24 to 31° C. and was maintained for 4 h. The n-heptane was removed under reduced pressure (30° C./3 h/20 mmHg). The conversion of the OH groups and the formation of the ester was confirmed by means of ¹H NMR spectroscopy.

A brownish wax having the following structure was obtained:

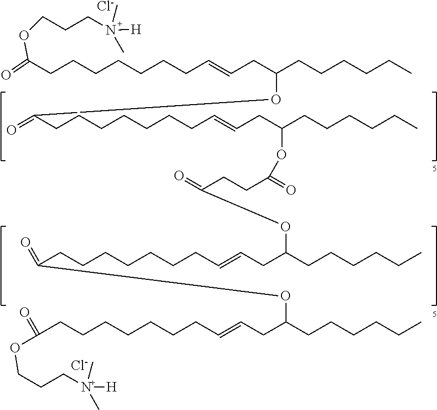

### Example 29c

Synthesis of a Glycerol Based Quat Using the Amine Salt of a Tertiary Amino Alcohol Ester of the [(Ricinoleic Acid)₆-Succinic Acid-(Ricinoleic Acid)₆] Diacid Estolide

In a 100 ml three-necked bottle, equipped with refluxing condenser, thermometer and mechanic stirrer 0.75 g (0.00549 mol epoxy groups; specific epoxy content 0.00729 mol epoxy groups/1 g) glycerol diglycidyl ether, 10.23 g (0.00275 mol) of the amine salt from synthesis example 29b, 50 g 2-propanol and 0.1 g deionized water are mixed at room temperature. The mixture is heated to 800° C. for 8 h. Afterwards, volatiles are removed under reduced pressure (40° C./2 h/20 mmHg). The complete conversion of the epoxy groups was confirmed by means of ¹H NMR spectroscopy.

A brownish wax having the following approximate structure is obtained:

The quat has an -[A-B]ₙ- structure with

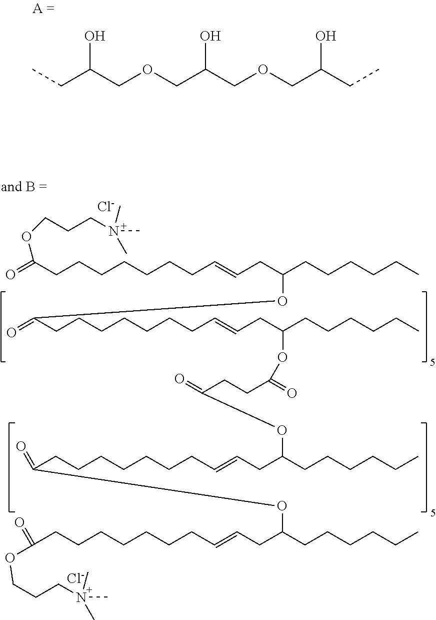

The number of repeating units n in the structural formula -[A-B]ₙ- of the compound obtained by the above procedure is within the range of n≤30.

### Application Tests

### Combing Force Measurements

Combing force measurements were carried out in order to quantify the effect of the compounds according to the invention. A Miniature Tensile Tester 175 (Dia-Stron Limited) was used.

Two different types of hair (Kerling International) were selected for these measurements:

| Hair type | Hair finishing method |
|---|---|
| White buffalo belly hair, 20 cm long | 1 |
| “buffalo hair” | |
| Euro-Hair, bleached heavily, 20 cm long | 2 |
| “damaged human hair” | |

### Hair Finishing Method 1 (Buffalo Hair)

The weight of the portion of the hair tresses to be finished is determined and the calculated total amount on active substance (based on the target mg active/1 g buffalo hair) dissolved in 2-propanol. The amount on 2-propanol used was calculated by the following formula:

m₂₋ₚᵣₒₚₐₙₒₗ(g)=1.94×m_{hair finished}

The 2-propanol solutions are evenly distributed over the hair tresses. The tresses are air dried for 2 h and further processed as outlined in the general protocol.

### Hair Finishing Method 2 (Damaged Human Hair)

The weight of the portion of the hair tresses to be finished is determined and the calculated total amount on active substance (based on the target mg active/1 g buffalo hair) dissolved in 2-propanol. The amount on 2-propanol used was calculated by the following formula:

m₂₋ₚᵣₒₚₐₙₒₗ(g)=0.64×m_{hair finished}

The 2-propanol solutions are evenly distributed over the hair tresses. The tresses are air dried for 2 h and further processed as outlined in the general protocol.

### General Protocol for the Pretreatment and Handling of Hair Tresses

Individual hair tresses (2.5 cm) were cut off from the respective stock tress and equilibrated in a humidity chamber at 50% relative humidity (rel. hum.) for 12 h. Afterwards, the dry tear off force and the wet average force (tresses rinsed with 38° C. tap water for 30 seconds) were determined for the untreated tresses (baseline measurements). Three strokes were carried out. The force data of the third stroke were used for the calculations.

The tresses were air dried and equilibrated in the climate chamber for additional 15 h. Afterwards, they were finished with the 2-propanol solutions as outlined for the hair finishing methods 1 and 2, air dried for two hours and equilibrated in the climate chamber for additional 15 h. Finally, the dry tear off force and the wet average force (tresses rinsed with 38° C. tap water for 30 seconds) were determined for the finished tresses (measurements finished hair). Three strokes were carried out. The force data of the third stroke were used for the calculations.

The ratio between the required combing force before finishing (baseline measurements) and the combing force after finishing (measurements finished hair) describes the effectiveness of a conditioning agent.

The following formula was used to calculate the relative combing force reduction:

Force reduction (%)=(Force_{baseleine}−Force_{finished})×100/Force_{baseline}

### Results Combing Force Measurements

### Buffalo Hair

| | | concentration active | dry tear | wet |
|---|---|---|---|---|
| | compound | (mg active/1 g | off force | average force |
| run | example | buffalo hair) | (reduction %) | (reduction %) |
| 1 | 6 | 2 | 50 | 67.3 |
| 2 | 6 | 5 | 43.6 | 37.2 |
| 3 | 6 | 10 | 2.2 | 24.6 |
| 4 | 6 | 20 | 28.9 | 17.3 |
| 6 | 9 | 5 | 51.7 | 20.3 |
| 7 | 11 | 5 | 28.6 | 66.7 |
| 8 | 13 | 5 | 17.1 | 56.7 |
| 9 | 16 | 5 | 46.6 | 49.9 |
| 10 | 17 | 5 | 28.4 | 20.3 |
| 11 | 20 | 5 | 16.2 | 51.2 |
| 12 | 21 | 5 | 50.6 | 46.0 |

### Damaged Human Hair

| | | concentration active | dry tear | wet |
|---|---|---|---|---|
| | | (mg active/1 g | off force | average force |
| run | compound | damaged human hair) | (reduction %) | (reduction %) |
| 13 | 6 | 5 | 88.5 | 69.2 |
| 14 | 10 | 5 | 84.7 | 60.9 |
| 15 | 11 | 5 | 90.9 | 72.3 |
| 16 | 11a | 5 | 88.0 | 77.4 |
| 17 | 11b | 5 | 88.4 | 94.6 |
| 18 | 11c | 5 | 70.1 | 83.2 |
| 19 | 12 | 5 | 71.1 | 89.6 |
| 20 | 13 | 5 | 62.7 | 87.8 |
| 21 | 17 | 5 | 83.8 | 91.2 |
| 22 | 21 | 5 | 92.8 | 88.7 |
| 23 | 29c | 5 | 90.0 | 83.0 |

The data in the above two tables on buffalo hair and damaged human hair show that the compounds according to the invention are able to reduce the combing forces on different keratinous substrates.

A comparison of the data for compounds 6, 17 and 21 on buffalo hair and damaged human hair highlights the specific effectiveness of the inventive compounds on human hair.

The data for compounds 11a, 11b and 11c demonstrate that mixed poly fatty acid sequences consisting of saturated as well as unsaturated fatty acids are effective on human hair.

The data for compounds 29c demonstrate that in addition to monofunctional poly fatty acid moieties terminated by monofunctional fatty acids (i.e. oleic acid, stearic acid) also di- and higher functional poly fatty acid moieties (i.e. located within the polymer chain) are effective on human hair.

### Synthesis Example 30

### Synthesis of a Di-Quat Using a Chloro Acetic Acid Ester Derivative of a Castor Oil Based Estolide

In a 500 ml three-necked bottle, equipped with refluxing condenser, thermometer, mechanical stirrer, dropping funnel and gas outlet tube 117.77 g (0.1262 mol) castor oil were placed at room temperature. Upon stirring, 14.25 g (0.1262 mol) chloro acetic acid chloride were added over 10 minutes. The temperature increased during the addition to 34° C. Afterwards, the temperature was increased to 80° C. for 1 h. The formation of the chloro acetic acid ester was confirmed by means of ¹H NMR spectroscopy.

75.93 g (0.2523 mol) oleic acid chloride were added during 20 minutes. Afterwards, the temperature was maintained at 80° C. for 2 hrs. Volatiles were removed under reduced pressure (80° C./2 h/20 mmHg). The complete conversion of the OH groups of the castor oil molecule and the formation of additional ester moieties was confirmed by means of ¹H NMR spectroscopy.

A brownish, transparent oil essentially having the approximate structure was obtained:

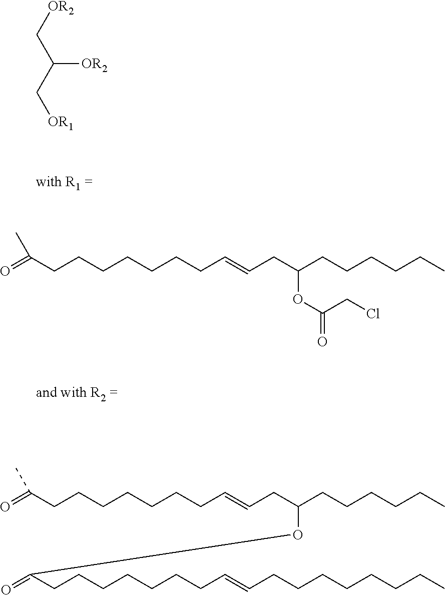

185 g 2-propanol and 10.87 g N,N,N′,N′-tetramethyl-1,6-hexanediamine (0.0631 mol) were added and the mixture heated to 800° C. for 27 hrs.

The complete conversion of the CH₂Cl groups was confirmed by means of ¹H NMR spectroscopy.

A brownish liquid containing a di-quat having the following structure was obtained:

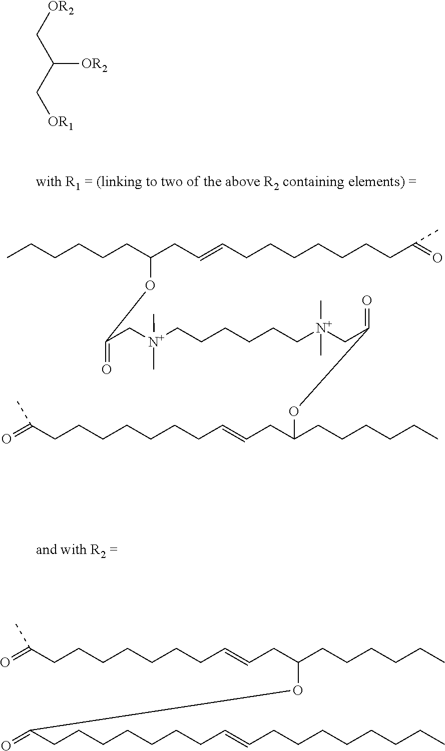

### Synthesis Example 30a

### Synthesis of a Di-Quat Using a Chloro Acetic Acid Ester Derivative of a Castor Oil Based Estolide and Having Oleate Counter Ions

In a 500 ml beaker equipped with a magnetic stirrer 100 g (0.000323 mol Cl⁻/1 g solution) of the solution from example 30, 9.85 g sodium oleate (0.0323 mol) and 5.5 g DI water were mixed for 1 h.

A second mixture of the same composition was prepared.

The two mixtures were unified and the volatiles removed (40° C./20 mbar/4 hrs).

A brownish viscous material having the following structure was obtained:

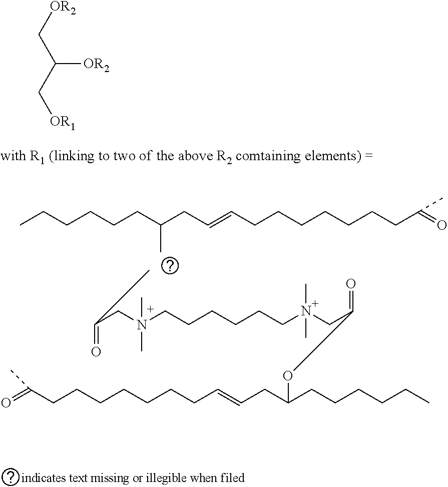

having two anionic counter ions

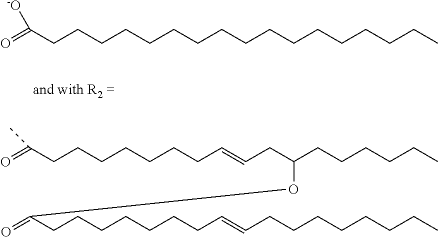

The formed sodium chloride precipitates upon storage.

### Synthesis Example 30b

### Synthesis of a Di-Quat Using a Chloro Acetic Acid Ester Derivative of a Castor Oil Based Estolide and Having Ricinoleic Acid-Oleic Acid Dimer Based Counter Ions

In a 500 ml beaker equipped wit a magnetic stirrer 100 g (0.000323 mol Cl⁻/1 g solution) of the solution from example 30, 18.19 g (0.0323 mol) of the ricinoleic acid-oleic acid dimer from synthesis example 1, 5.5 g DI water and 1.16 g NaOH were mixed for 1 h.

A second mixture of the same composition is prepared.

The two mixtures were unified and the volatiles removed (40° C./20 mbar/4 hrs).

A brownish viscous material having the following structure is obtained:

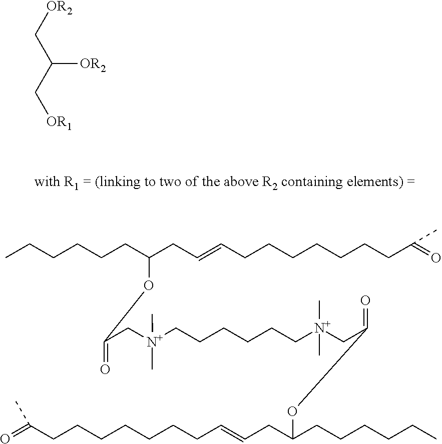

having two anionic counter ions

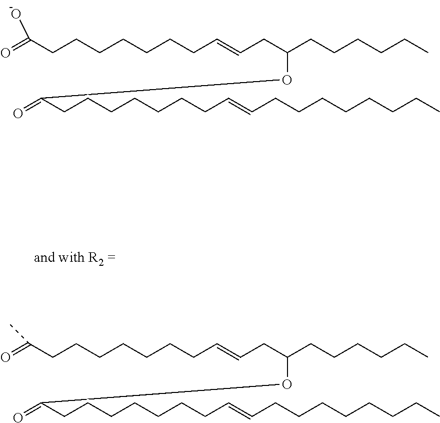

The formed sodium chloride precipitates upon storage.

### Example 31

### Synthesis of a Hexa-Tertiary Amine

In a 1000 ml three-necked bottle, equipped with refluxing condenser, thermometer and magnetic stirrer, N2 inlet/outlet 22.89 g (0.1669 mol epoxy groups; specific epoxy content 0.00729 mol epoxy groups/1 g) glycerol diglycidyl ether, 31.26 g (0.1669 mol NH groups) (CH₃)₂NCH₂CH₂CH₂NHCH₂CH₂CH₂N(CH₃)₂ and 450 g propylene glycol mono methyl ether were mixed at room temperature. The mixture was heated to 110° C. for 8 hrs. The complete conversion of the epoxy groups was confirmed by means of ¹H NMR spectroscopy.

A 10.74% active brownish solution containing 0.99299 mmol amine/1 g solution was obtained.

Approximate structure of the hexa-tertiary amine:

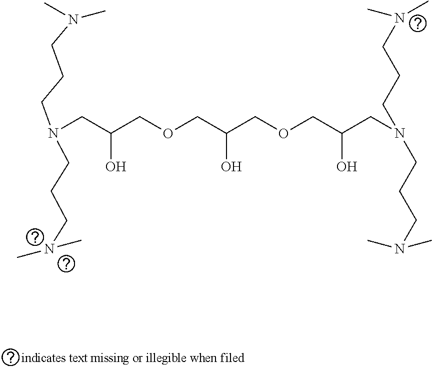

### Example 31a

### Synthesis of a Hexa-Tertiary Amine

In a 500 ml three-necked bottle, equipped with refluxing condenser, thermometer and magnetic stirrer, N2 inlet/outlet 11.83 g (0.1169 mol epoxy groups) 1,4-butanediol diglycidyl ether, 21.91 g (0.1169 mol NH groups) (CH₃)₂NCH₂CH₂CH₂NHCH₂CH₂CH₂N(CH₃)₂ and 221.87 g propylene glycol mono methyl ether were mixed at room temperature. The mixture was heated to 115° C. for 10 hrs. The complete conversion of the epoxy groups was confirmed by means of ¹H NMR spectroscopy.

A yellowish solution containing 1.3815 mmol amine/1 g solution was obtained.

Approximate structure of the hexa-tertiary amine:

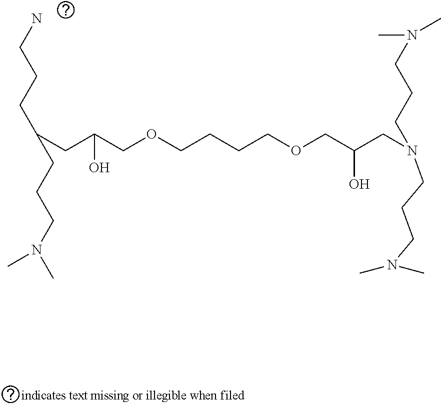

### Synthesis Example 32

### Synthesis of a Tetra-Quat Using a Chloro Acetic Acid Ester Derivative of a Castor Oil Based Estolide

In a 1000 ml three-necked bottle, equipped with refluxing condenser, thermometer, mechanical stirrer, N2 inlet/outlet 216 g of the solution from example 31, 220 g (0.143 mol) of the castor oil based chloro acetic acid ester intermediate described in example 30 and 55.2 g propylene glycol mono methyl ether were mixed at room temperature. The temperature was increased to 115° C. for 25 hrs. Afterwards, volatiles were removed under reduced pressure (60° C./20 mbar/4 hrs).

A brownish, transparent vicous oil essentially having the approximate structure was obtained:

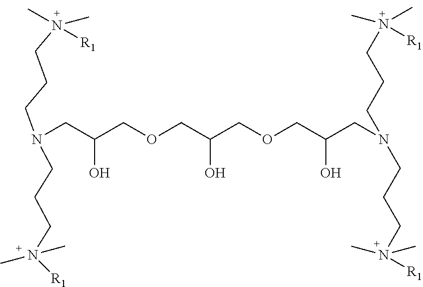

having four anionic counter ions Cl⁻
with

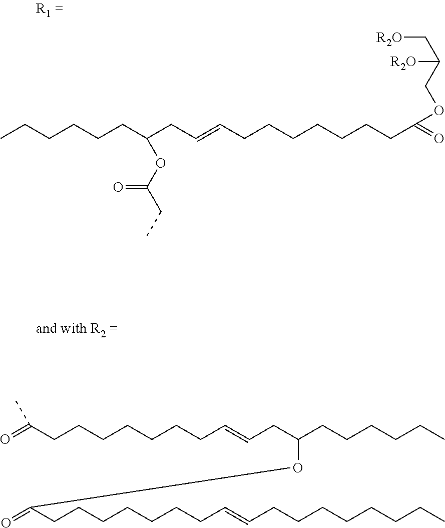

### Synthesis Example 32a

### Synthesis of a Tetra-Quat Using a Chloro Acetic Acid Ester Derivative of a Castor Oil Based Estolide and Having Ricinoleic Acid-Oleic Acid Dimer Counter Ions

In a 500 ml one neck bottle 100 g of the tetra-quat from example 32, 32.60 g (0.057906 mol COOH) ricinoleic acid-oleic acid dimer from synthesis example 1, 2.08 g NaOH and 5.5 g DI water were placed. The composition was mixed on a rotavap for 2 hrs at atmospheric pressure.

Afterwards, volatiles were removed at 40° C./20 mbar/2 hrs.

A brownish viscous material having the following approximate structure was obtained:

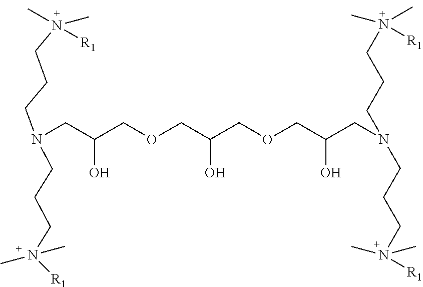

having four anionic counter ions

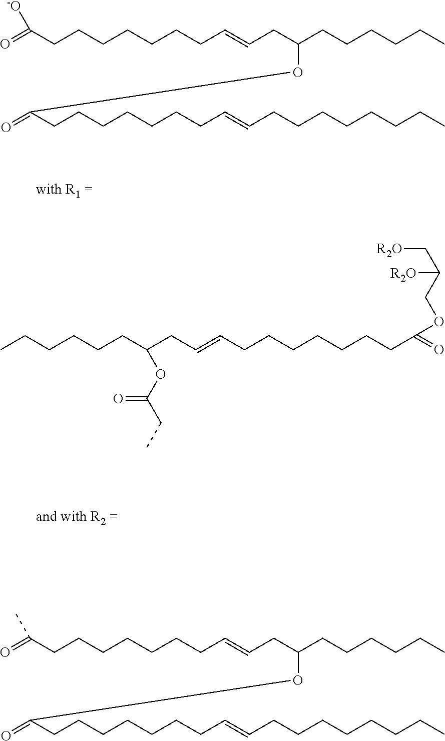

The formed sodium chloride precipitates upon storage.

### Synthesis Example 32b

### Synthesis of a Tetra-Quat Using a Chloro Acetic Acid Ester Derivative of a Castor Oil Based Estolide and Having Additional Amine Salt Groups

In a 500 ml one neck bottle 100.40 g of the tetra-quat from example 32 (0.02906 mol tertiary amine), 16.36 g (0.02906 mol COOH) ricinoleic acid-oleic acid dimer from synthesis example 1, and 5.5 g DI water were placed. The composition was mixed on a rotavap for 2 hrs at atmospheric pressure. Afterwards, volatiles were removed at 40° C./20 mbar/2 hrs.

A brownish viscous material having the following approximate structure is obtained:

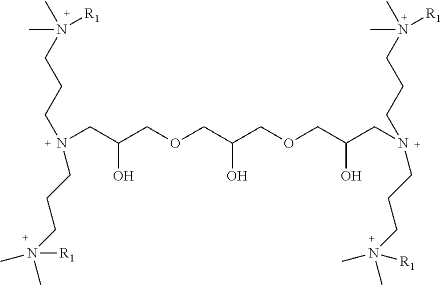

having four anionic counter ions Cl⁻
and two anionic counter ions

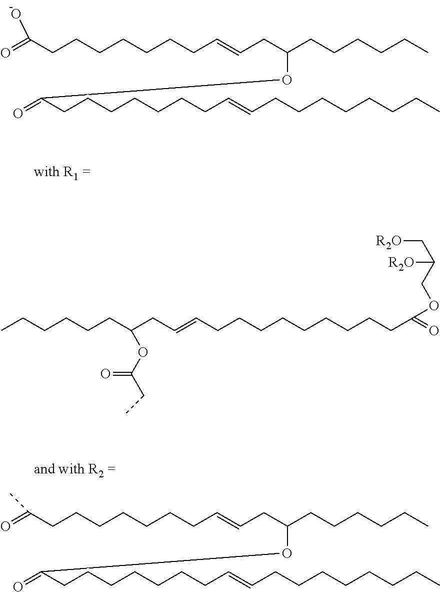

### Example 33

### Synthesis of a Di-Quat Using a Chloro Acetic Acid Ester Derivative of a Castor Oil Based Estolide

In a 1000 ml three-necked bottle, equipped with refluxing condenser, thermometer, mechanical stirrer, N₂ inlet/outlet 216 g of the solution from example 31, 145.95 g (0.0695 mol) of the castor oil based chloro acetic acid ester intermediate described in synthesis example 7, and 168 g propylene glycol mono methyl ether were mixed at room temperature. The temperature was increased to 1150° C. for 32 hrs. Afterwards, volatiles were removed under reduced pressure (60° C./20 mbar/3 hrs).

A brownish, transparent vicous oil essentially having the approximate structure was obtained:

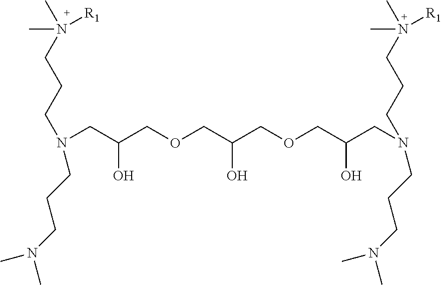

having two anionic counter ions Cl⁻
with

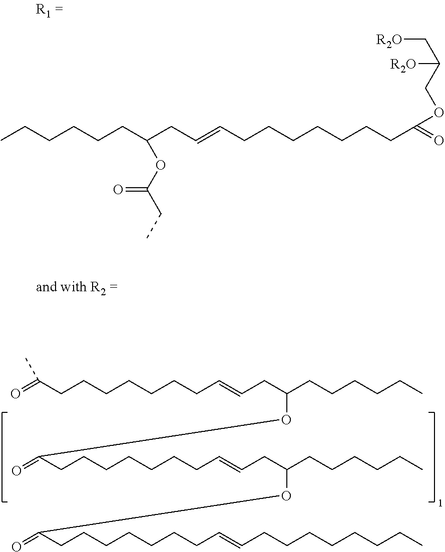

### Synthesis Example 33a

### Synthesis of a Di-Quat Using a Chloro Acetic Acid Ester Derivative of a Castor Oil Based Estolide and Having Two Additional Amine Salt Groups

In a 500 ml one neck bottle 86.36 g of the di-quat from example 33, 19.99 g (0.0355 mol COOH) ricinoleic acid-oleic acid dimer from synthesis example 1 and 5.5 g DI water were placed. The composition was mixed on a rotavap for 2 hrs at atmospheric pressure.

Afterwards, volatiles were removed at 40° C./20 mbar/2 hrs.

A brownish viscous material having the following approximate structure was obtained:

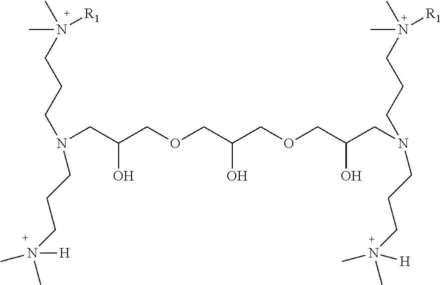

having two anionic counter ions Cl⁻ and
two anionic counter ions

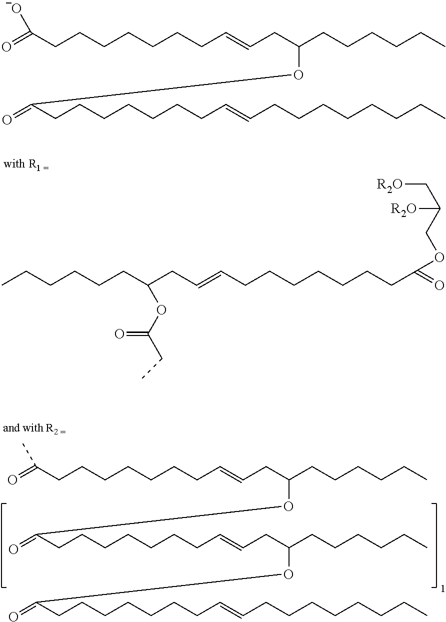

### Synthesis Example 33b

### Synthesis of a Di-Quat Using a Chloro Acetic Acid Ester Derivative of a Castor Oil Based Estolide and Having Four Additional Amine Salt Groups

In a 500 ml one neck bottle 73.45 g of the di-quat from example 33, 34.01 g (0.0604 mol COOH) ricinoleic acid-oleic acid dimer from synthesis example 1 and 4.04 g DI water were placed. The composition was mixed on a rotavap for 2 hrs at atmospheric pressure.

Afterwards, volatiles were removed at 40° C./20 mbar/2 hrs.

A brownish viscous material having the following approximate structure is obtained:

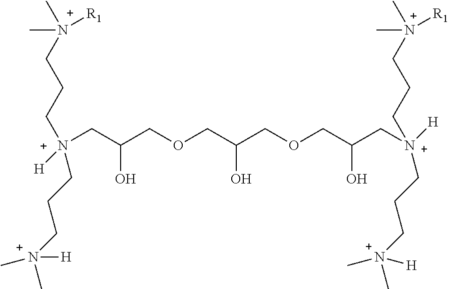

having two anionic counter ions Cl⁻ and
four anionic counter ions

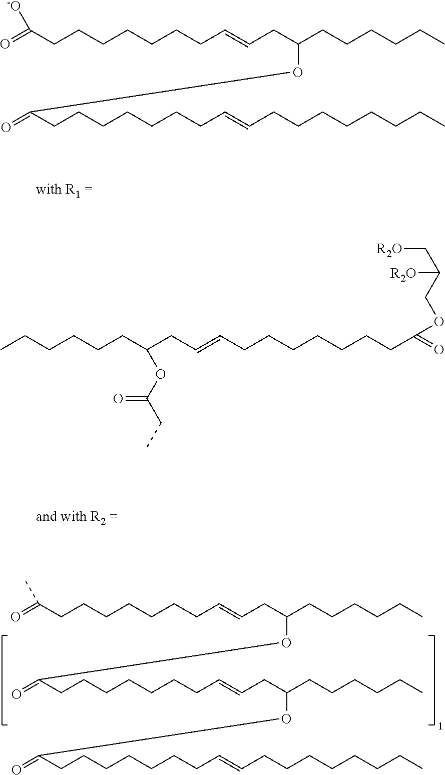

### Example 34

### Synthesis of a Di-Quat Using a Chloro Acetic Acid Ester Derivative of a Castor Oil Based Estolide

In a 1000 ml three-necked bottle, equipped with refluxing condenser, thermometer, mechanical stirrer, N2 inlet/outlet 217.19 g of the solution from example 31a, 210 g (0.1 mol) of the castor oil based chloro acetic acid ester intermediate described in synthesis example 7, and 50 g propylene glycol mono methyl ether were mixed at room temperature. The temperature was increased to 1150° C. for 34 hrs. Afterwards, volatiles were removed under reduced pressure (60° C./20 mbar/4 hrs).

A brownish, transparent vicous oil essentially having the approximate structure was obtained:

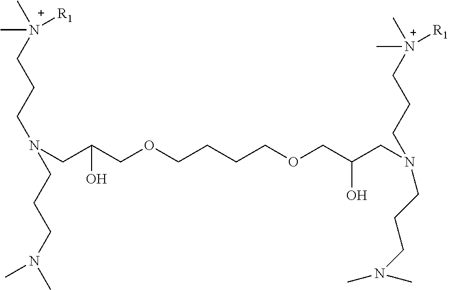

having two anionic counter ions Cl⁻
with

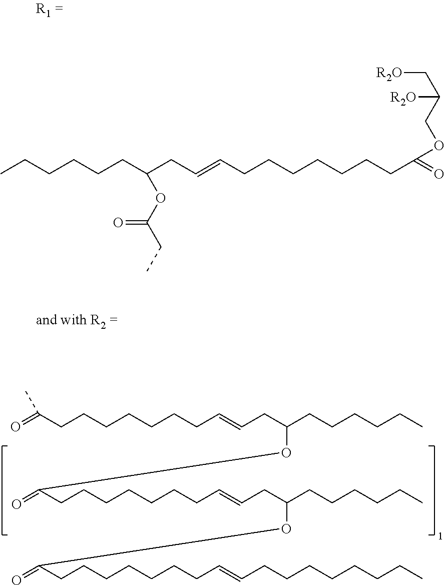

### Synthesis Example 34a

### Synthesis of a Tetra-Quat Using a Chloro Acetic Acid Ester Derivative of a Castor Oil Based Estolide and Having Two Additional Oleate Amine Salt Groups

In a 500 ml one neck bottle 116, 13 g of the di-quat from example 34, 13.29 g (0,047 mol COOH) oleic acid and 6.39 g DI water were placed. The composition was mixed on a rotavap for 2 hrs at atmospheric pressure. Afterwards, volatiles were removed at 40° C./20 mbar/2 hrs.

A brownish viscous material having the following approximate structure was obtained:

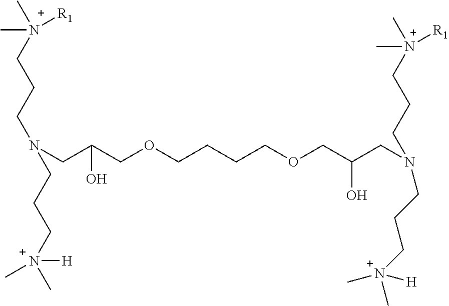

having two anionic counter ions Cl⁻ and
two anionic counter ions

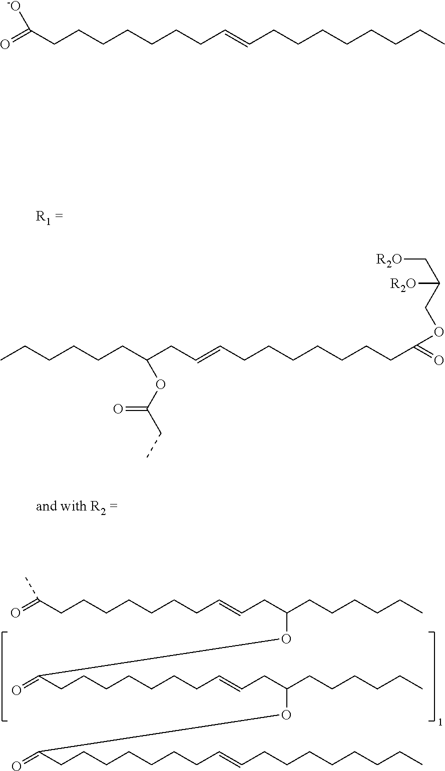

### Synthesis Example 34b

### Synthesis of a Tetra-Quat Using a Chloro Acetic Acid Ester Derivative of a Castor Oil Based Estolide and Having Two Additional Ricinoleic Acid-Oleic Acid Dimer Amine Salt Groups

In a 500 ml one neck bottle 110.47 g of the di-quat from example 34, 25.19 g (0.0447 mol COOH) of the ricinoleic acid-oleic acid dimer from synthesis example 1 and 6.08 g DI water were placed. The composition was mixed on a rotavap for 2 hrs at atmospheric pressure. Afterwards, volatiles were removed at 40° C./20 mbar/2 hrs.

A brownish viscous material having the following approximate structure is obtained:

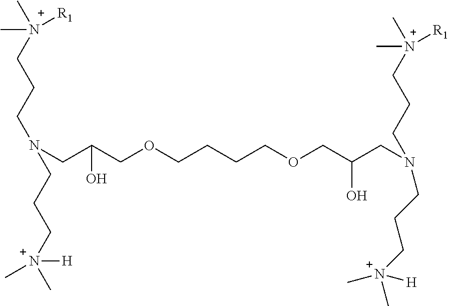

having two anionic counter ions Cl⁻ and
two anionic counter ions

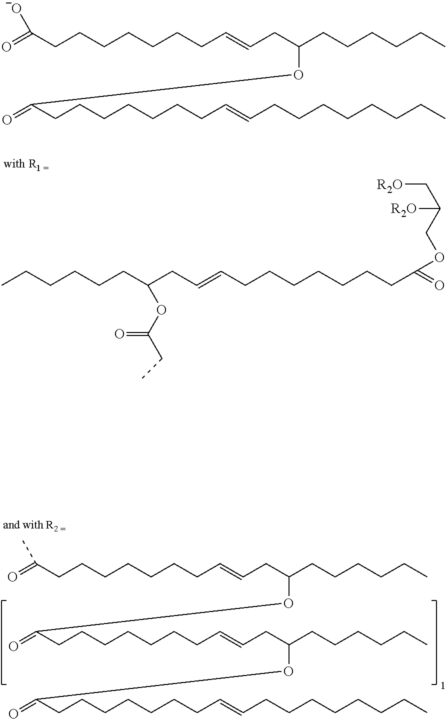

### Example 35

### Synthesis of a Di-Quat Using a Chloro Acetic Acid Ester Derivative of a Castor Oil Based Estolide

In a 1000 ml three-necked bottle, equipped with refluxing condenser, thermometer, mechanical stirrer, N₂ inlet/outlet 120.36 g of the solution from example 31, 167.62 g (0.0797 mol) of the castor oil based chloro acetic acid ester intermediate described in synthesis example 7a and 80.6 g propylene glycol mono methyl ether were mixed at room temperature. The temperature was increased to 115° C. for 31 hrs. Afterwards, volatiles were removed under reduced pressure (60° C./15 mbar/1.5 hrs).

A brownish waxy material having the following approximate structure was obtained:

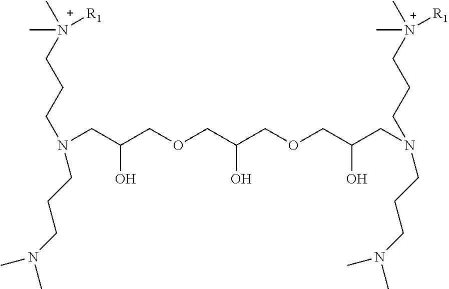

having two anionic counter ions Cl⁻
with R₁=

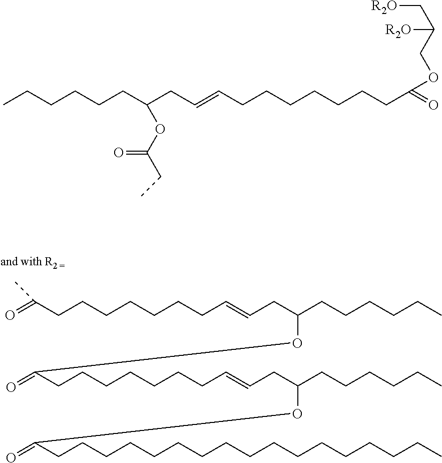

### Synthesis Example 35a

### Synthesis of a Di-Quat Using a Chloro Acetic Acid Ester Derivative of a Castor Oil Based Estolide and Having Two Additional Amine Salt Groups

In a 500 ml one neck bottle 85.42 g of the di-quat from example 35, 10.4 g (0.0184 mol COOH) ricinoleic acid-stearic acid dimer from synthesis example 2a and 4.7 g DI water were placed. The composition was mixed on a rotavap for 2 hrs at atmospheric pressure. Afterwards, volatiles were removed at 40° C./20 mbar/2 hrs.

A brownish waxy material having the following approximate structure was obtained:

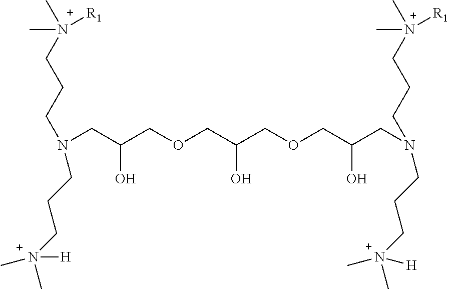

having two anionic counter ions Cl⁻ and
two anionic counter ions

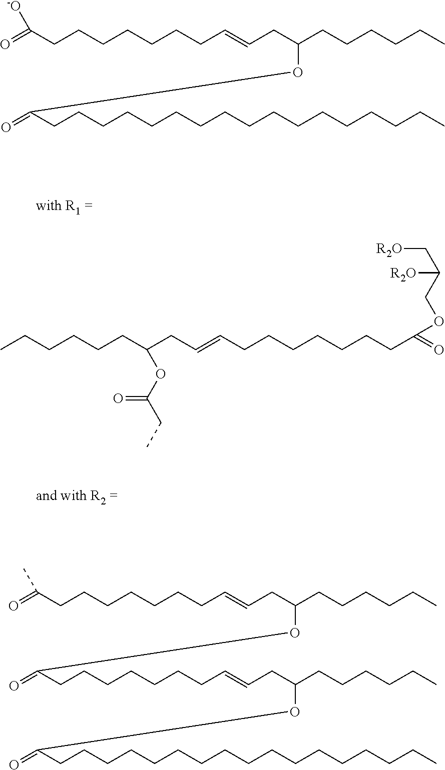

### Synthesis Example 35b

### Synthesis of a Di-Quat Using a Chloro Acetic Acid Ester Derivative of a Castor Oil Based Estolide and Having Two Additional Amine Salt Groups

In a 500 ml one neck bottle 89.30 g of the di-quat from example 35, 5.43 g (0.01924 mol COOH) oleic acid and 4.7 g DI water were placed. The composition was mixed on a rotavap for 1 h at atmospheric pressure. Afterwards, volatiles were removed at 40° C./20 mbar/2 hrs.

A brownish waxy material having the following approximate structure is obtained:

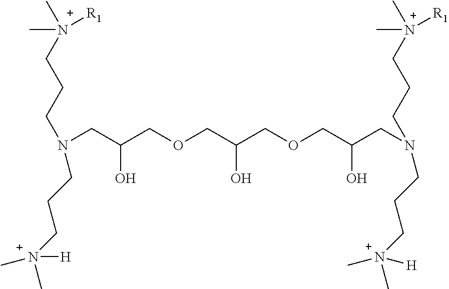

having two anionic counter ions Cl⁻ and
two anionic counter ions

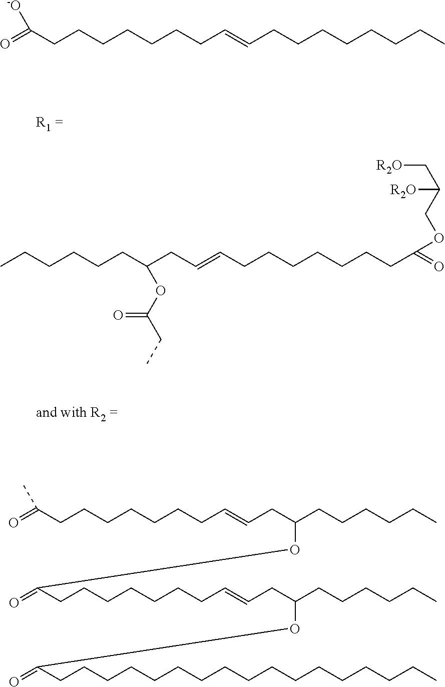

### Examples of Scalable Processes Example 36

### Synthesis of a (Ricinoleic Acid-Stearic Acid) Dimer Acid Containing Mixture

In a 500 ml three-necked bottle, equipped with refluxing condenser, thermometer and magnetic stirrer, dropping funnel, N₂ inlet tube and vacuum outlet, 163.45 g (0.5746 mol) stearic acid are heated to 70° C. A gentle N₂ stream flushed through the bottle during the course of the reaction. 81.66 g (0.2736 mol) of a ricinoleic acid containing 15% oleic acid and having a degree on free OH groups of 70% of the theoretical value were added. The mixture was heated to 160° C. for 1 h. Additional 163.34 g (0.5417 mol) of the ricinoleic acid were added during 1 hr at 160° C. and the temperature maintained for 5 hrs. Afterwards, the temperature was increased to 200° C. and maintained for 17 hrs.

The complete conversion of the OH groups was confirmed by means of H NMR spectroscopy.

A grey-brownish wax containing as main component the ricinoleic acid-stearic acid dimer was obtained.

Ricinoleic acid-stearic acid dimer (main product approx. 80%)

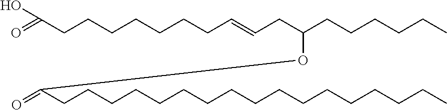 [SMILES: CCCCCCCCCCCCCCCCCC(=O)OC(C/C=C/CCCCCCCC(=O)O)CCCCCC]

Ricinoleic acid-ricinoleic acid-stearic acid trimer (approx. 10%)

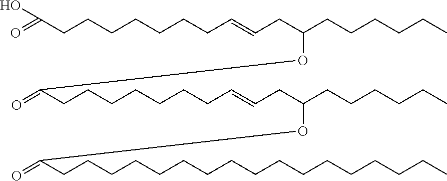 [SMILES: CCCCCCCCCCCCCCCCCC(=O)OC(C/C=C/CCCCCCCC(=O)OC(C/C=C/CCCCCCCC(=O)O)CCCCCC)CCCCCC]

Additionally in total approx. 10% of acids of the type (ricinoleic acid)₃₋₆-stearic acid, ricinoleic acid dimer, ricinoleic acid, stearic acid.

### Example 36a

### Synthesis of a Chloro Acetic Acid Ester Derivative of a Castor Oil Based Estolide

In a 100 ml three-necked bottle, equipped with refluxing condenser, thermometer, magnetic stirrer, dropping funnel, N₂ inlet and gas outlet tube 30 g (0.0321 mol) castor oil were placed and heated to 80° C. A gentle N₂ stream flushed through the bottle during the course of the reaction. Upon stirring, 4.25 g (0.0449 mol) chloro acetic acid were added. The temperature was increased to 120° C. and maintained for 6 hrs. Afterwards, the temperature was increased to 140° C. and maintained there for 5 hrs. The formation of approx. 0.85 chloro acetic acid ester bonds per castor oil molecule was confirmed by means of ¹H NMR spectroscopy. 0.46 g chloro acetic acid (0.0048 mol) were added and the reaction continued at 140° C. for additional 8 hrs.

The formation of approx. 1 chloro acetic acid ester bond per castor oil molecule was confirmed by means of ¹H NMR spectroscopy.

Excess chloro acetic acid was removed under reduced pressure (140° C./25 mbar).

36.31 g (0.0643 mol) (ricinoleic acid)₁-stearic acid dimer acid from synthesis example 36 were added. The temperature was increased to 200° C. and maintained for 17 hrs.

The complete conversion of the OH groups of the castor oil molecule and the formation of additional ester moieties was confirmed by means of ¹H NMR spectroscopy.

A brownish wax like material essentially having the approximate structure was obtained:

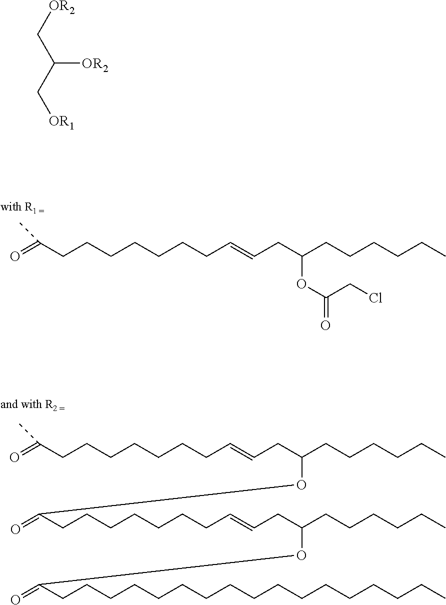

### Example 36b

### Synthesis of a Tetra-Quat Using a Chloro Acetic Acid Ester Derivative of a Castor Oil Based Estolide

In a 100 ml three-necked bottle, equipped with refluxing condenser, thermometer, mechanical stirrer 16, 75 g (0.0166 mol tertiary amine) of the tertiary amino solution from example 31, 23, 33 g (0.0111 mol Cl) of the castor oil based chloro acetic acid ester intermediate described in example 36a and 10, 18 g propylene glycol mono methyl ether were mixed at room temperature. The temperature was increased to 115° C. for 24 hrs. Afterwards, volatiles were removed under reduced pressure (60° C./15 mbar/1.5 hrs).

A brownish waxy material having the following approximate structure was obtained:

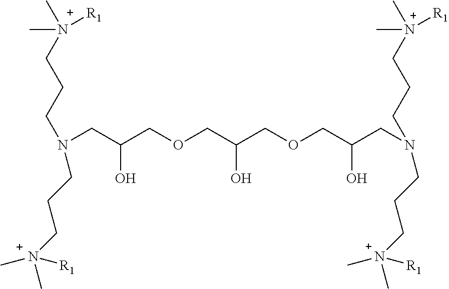

having four anionic counter ions Cl⁻

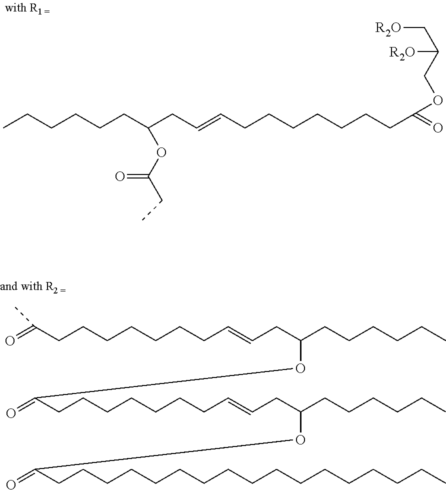

### Synthesis Example 37a

Synthesis of a (Ricinoleic Acid₂-Butanediol-Ricinoleic Acid₂) Pentamer Diol

In a 100 ml three-necked bottle, equipped with refluxing condenser, thermometer and magnetic stirrer, dropping funnel, N₂ inlet tube and gas/vacuum outlet, 7.22 g (0.080 mol) 1,4-butane diol and 47.82 g (0.16 mol) ricinoleic acid were mixed and heated to 160° C. for 17 hrs. A gentle N₂ stream was passed through the gas volume above the liquid's surface.

42.16 g (0.065 mol) of the received intermediate of the approximate structure

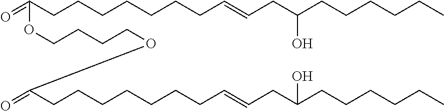 [SMILES: CCCCCCC(O)C/C=C/CCCCCCCC(=O)OCCCCOC(=O)CCCCCCC/C=C/CC(O)CCCCCC]

and 38.66 g (0.13 mol) ricinoleic acid are mixed in a separate bottle and heated to 160° C. for 10 hrs. Afterwards, the temperature was increased to 200° C. for 6 hrs. A gentle N₂ stream was passed through the gas volume above the liquid's surface in the course of this reaction.

A yellow-brownish liquid containing the following averaged structure as main component was obtained.

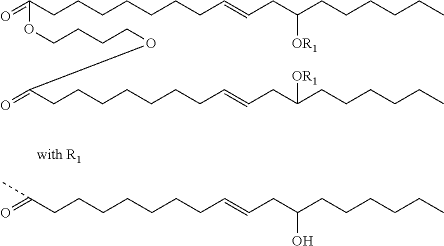

### Example 37b

Synthesis of a Di-Quat Using a Chloro Acetic Acid Ester Derivative of a (Ricinoleic Acid₂-Butanediol-Ricinoleic Acid₂) Pentamer Diol

In a 50 ml three-necked bottle, equipped with refluxing condenser, thermometer, mechanical stirrer, N₂ inlet/outlet 2.07 g (1.708 mmol) of the 1,4-butane diol based pentamer diol from example 37a and 0.44 g (3.928 mmol) chloroacetic acid chloride were mixed at room temperature. The temperature was increased to 700° C. for 5 hrs. Afterwards, volatiles were removed under reduced pressure (70° C./15 mbar/1 h).

An intermediate of the following averaged structure was obtained

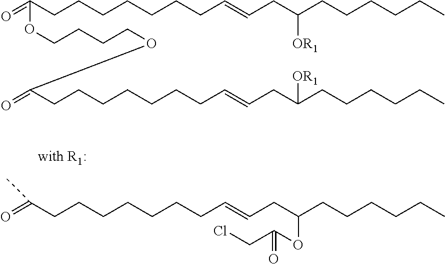

0.59 g (3.416 mmol) N,N,N′,N′-tetramethyl-1,6-hexanediamine and 17.57 g propylene glycol mono methyl ether were added and the reaction continued at 115° C. for 20 hrs. Volatiles were removed under reduced pressure (60° C./15 mbar/1 h). A quantitative conversion of the CH₂Cl groups was determined by means of 1H-NMR spectroscopy.

A yellow liquid-waxy material having the following approximate structure was obtained:

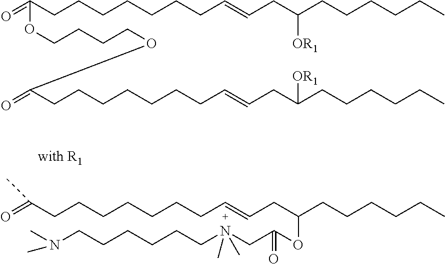

and having two anionic counter ions Cl⁻.

### Further Application Tests

### Damaged Human Hair

| | | concentration active | dry tear | wet |
|---|---|---|---|---|
| | compound | (mg active/1 g | off force | average force |
| run | example | damaged human hair) | (reduction %) | (reduction %) |
| 24 | 30b | 5 | 77.0 | 73.0 |
| 25 | 32a | 5 | 45.2 | 79.5 |
| 26 | 33a | 5 | 39.0 | 76.1 |
| 27 | 33b | 5 | 52.0 | 84.7 |
| 28 | 34a | 5 | 61.9 | 81.5 |
| 29 | 34b | 5 | 51.5 | 78.2 |
| 30 | 35a | 5 | 73.9 | 60.5 |
| 31 | 35b | 5 | 80.0 | 88.7 |

The data in the above two tables on damaged human hair show that the compounds according to the invention are able to reduce the combing forces on different keratinous substrates. The data for compounds 30b and 32a on damaged human hair highlight the effectiveness of inventive materials having poly fatty adid moieties in the cation as well as simultaneously in the anion as replacements for inorganic anions. The data on compounds 33a, 33b, 34a and 34b highlight the value of amine salt groups in addition to quat groups when applied on damaged human hair. The data on compounds 35a and 35b highlight the value of structures bearing higher melting moieties in the poly fatty chain.

### Hair Conditioner Application Tests:

### Hair Conditioner Formulation

| | Conditioner | Example | Example |
|---|---|---|---|
| Chemical Name/INCI Name | base* | 34b* | 35a* |
| Phase A | | | |
| Cetearyl alcohol | 2 | 2 | 2 |
| Stearyl Alcohol | 3.6 | 3.6 | 3.6 |
| Stearamidopropyl Dimethylamine | 1.9 | 1.9 | 1.9 |
| Phase B | | | |
| Aqua | q.s. to 100 | q.s. to 100 | q.s. to 100 |
| Lactic acid | 0.5 | 0.5 | 0.5 |
| Phase C | | | |
| 34b | 0 | 2 | 0 |
| 35a | 0 | 0 | 2 |
| Phase D | | | |
| DMDM hydantoin | 0.5 | 0.5 | 0.5 |

*All values of the amounts of the components given indicate “parts by weight based on 100 parts by weight of the total composition”

### Procedure:

Phase A and phase B were heated separately at 80° C. (Phase A) & 60° C. (Phase B), respectively. The Phase A was mixed in Phase B.
After addition, the mixture was stirred for 30 min at 60° C. Phase C was added and the reaction was brought to a temperature to 25° C. Phase D was stirred for 15 min. The composition was stored in a suitable container.

### Combing Analysis:

Each conditioner is evaluated in duplicates and the average as shown in the following data is considered for conclusion.

### Combing Force Measurement Procedure:

The combing force measurements were carried out using a Dia-Stron MTT 175 (Dia-Stron Limited) as described for above combing force measurements.
1. Asian hair tresses (2.5 gm) were prewashed with 2% NaOH followed by 10% SLES wash
2. The total work done in wet and dry combing was measured.
Therein “total work done” is defined as work done during the movement of the comb across the hair tress and measured by Dia-Stron MTT175.
3. The hair tresses were washed with 350 mg of conditioner followed by washing with warm water throughly
4. The total work done in wet and dry combing was measured
5. The % of work done reduction was calculated.
   Dry and Wet combing measurements: Total work done

| | Dry combing % | Wet combing % |
|---|---|---|
| | Reduction in | Reduction in |
| | total work done | total work done |
| | Treated vs. untreated | Treated vs. untreated |
| | (average two runs) | (average two runs) |
| cond. Base alone | 23 | 52 |
| cond. Base + ex. 34b | 33 | 75 |
| cond. Base + 35a | 37 | 73 |

The data show that the addition of the inventive compounds 34b and 35a to a conditioner formulation provides a dry and wet combing total work reduction which goes significantly beyond the reduction caused by the conditioner base alone.

### Hair Dry Friction Measurement

### Procedure:

1. Asian hair tresses (2.5 gm) were prewashed with 2% NaOH followed by 10% SLES wash
2. The hair tresses were dried thoroughly and equilibriated at 50% humidity and the dry friction was measured (CoF)
(The dry friction was measured by means of Tribometer instrument from CSM)
3. The hair tresses were washed with 350 mg of conditioner followed by washing with warm water thoroughly
4. The hair tresses were dried thoroughly and equilibriated at 50% humidity and the dry friction was measured (CoF)
5. The friction reduction (in %) was calculated

### % Friction Reduction:

| | Dry friction |
|---|---|
| | % Reduction in coefficient of |
| | friction (CoF) |
| | Treated vs. untreated |
| | (average of two runs) |
| cond. Base alone | 8 |
| cond. Base + 34b | 31 |
| cond. Base + 35a | 26 |

The data show that the addition of the inventive compounds 34b and 35a to a conditioner formulation provides a dry coeffcient of friction reduction which goes significantly beyond the reduction caused by the conditioner base alone.

1.-41. (canceled)

42. A compound of the general formula (I):
R¹(—F)ₓ (I),
wherein
x is 1 to 50,
R¹ is selected from x-valent, optionally substituted hydrocarbon radicals which have up to 1000 carbon atoms, and may contain optionally one or more groups selected from —O—, —NH—, —C(O)—, —C(S)—, tertiary amino groups 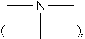
and can be substituted by one or more groups selected from OH groups and halide groups, and
F can be the same or different and is represented by the general formula (II) 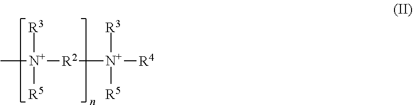
wherein the groups F bind to a carbon atom of R¹, and
n is independently 0 to 100,
R² can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups 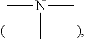
and can be substituted with one or more groups selected from OH groups and halide groups,
R³, R⁴, R⁵ can be the same or different and are selected from hydrogen and optionally substituted straight-chain, cyclic or branched, saturated, unsaturated or aromatic hydrocarbon radicals which have up to 1000 carbon atoms, which optionally contain one or more groups selected from —O—, —NH—, —C(O)—, —C(S)—, tertiary amino groups 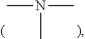
quaternary ammonium groups 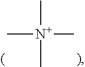
and can be substituted with one or more groups selected from OH groups and halide groups,
wherein R³, R⁴, R⁵ each bind with a carbon atom to the nitrogen atom,
the counter ions A⁻ of the ammonium ions are selected from mono to trivalent inorganic and mono- to 30000-valent organic anions, and
at least one of R¹, R², R³, R⁴, R⁵ present in the cationic structure of the general formulas (I) and (II) contains at least one moiety of the formulas (III) or (IV):
(—X—C(O)—R⁶)ₘ—X—C(O)— (III) or
(—C(O)—X—R⁶)ₘ—C(O)—X— (IV),
wherein
m=1 to 20,
X is O or NR¹¹
R¹¹ is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 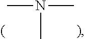
and can be substituted with one or more hydroxyl and halide groups,
R⁶ is independently selected from optionally substituted straight-chain, cyclic or branched, saturated or unsaturated hydrocarbon radicals which have 1 to 36 carbon atoms,
with the proviso that at least one R⁶ has more than 6 carbon atoms, and
that for x=1
R¹, R³, R⁴, R⁵ do not bind through —OCH₂CH₂— to the nitrogen atom of the group

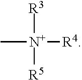

43. The compound according to claim 42, wherein x is 2 to 50.

44. The compound according to claim 42, which does not comprise a poly(ethylene oxide) or poly(propylene oxide) unit.

45. The compound according to claim 42, wherein R¹ contains at least one moiety of the general formula (IIIa)
(—X—C(O)—R⁶)ₘ—X—C(O)—R⁷ (IIIa),
or of the general formula (IVa)
(—C(O)—X—R⁶)ₘ—C(O)—X—R⁷ (IVa)
wherein X and R⁶ and m are as defined, and
R⁷ is independently selected from optionally substituted straight-chain, cyclic or branched, saturated or unsaturated hydrocarbon radicals which have 1 to 36 carbon atoms, optionally containing one or more groups selected from —O—, —NH— —C(O)—, —C(S)—, tertiary amino groups 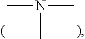
quaternary ammonium groups 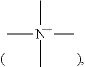
and which can be substituted with OH groups or halide groups, wherein the radical R⁷ cannot contain a combination of a —C(O)— group and a —O— group or a combination of a —C(O)— group and a —NH— or tertiary amino group forming an internal carboxylate group or an internal amide group.

46. The compound of claim 42, wherein only one or more of the residues R¹ or R² contain at least one moiety of the general formulas (III) or (IV)
(—X—C(O)—R⁶)ₘ—X—C(O)— (III) or
(—C(O)—X—R⁶)ₘ—C(O)—X— (IV),
wherein X, R⁶, and m are as defined.

47. The compound according to claim 42 wherein at least 1% of all groups F contain at least one moiety of the general formula (III) or (IV), or wherein at least 1% of all groups F contain at least one moiety of the general formula (IIIa) or (IVa)

48. The compound according to claim 42, wherein in formula (I) x is 2 and which is of the general formula (V): 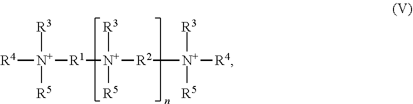
wherein R¹, R², R³, R⁴, R⁵ and n are as defined.

49. The compound according to claim 42, wherein F has the general formula (VI): 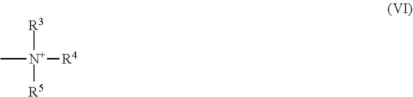
and the groups F bind to a carbon atom of R¹,
wherein
R³, R⁴, R⁵ are independently selected from hydrogen and optionally substituted straight-chain, cyclic or branched, saturated, unsaturated or aromatic hydrocarbon radicals which have up to 300 carbon atoms, and, which optionally contain one or more groups selected from —O—, —NH—, —C(O)—, —C(S)—, tertiary amino groups 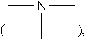
quaternary ammonium groups 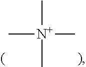
and can be substituted by OH,
the counter ions A⁻ are selected from mono- to trivalent inorganic anions and mono- to 30000-valent organic anions
with the proviso that at least one of the radicals R¹, R³, R⁴, R⁵ of the cationic structure of the formulas (I) and (II) contains at least one moiety of the general formulas (IIIa) or (IVa):
(—X—C(O)—R⁶)ₘ—X—C(O)—R⁷ (IIIa)
(—C(O)—X—R⁶)ₘ—C(O)—X—R⁷ (IVa)
wherein X is as defined above,
m=1 to 20, and
R⁶ is independently selected from optionally substituted straight-chain, cyclic or branched, saturated or unsaturated hydrocarbon radicals which have 1 to 36 carbon atoms,
R⁷ is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, tertiary amino groups 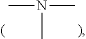
quaternary ammonium groups 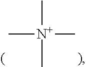
and which can be substituted with OH groups or halide groups, wherein the radical R⁷ cannot contain a combination of a —C(O)— group and a —O— group or a combination of a —C(O)— group and a —NH— or tertiary amino group forming an internal carboxylate group or an internal amide group,
with the proviso that at least one R⁶ has more than 6 carbon atoms, and
that for x=1
R¹, R³, R⁴, R⁵ do not bind through —OCH₂CH₂— to the nitrogen atom of the group

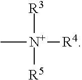

50. The compound according to claim 42
wherein R¹ is selected from monovalent to pentacontavalent, optionally substituted hydrocarbon radicals which have up to 1000 carbon atoms, and may contain optionally one or more groups selected from —O—, —NH—, —C(O)—, —C(S)—, tertiary amino groups 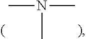
quaternary ammonium groups 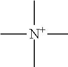
and can be substituted by —OH, and
F has the general formula (VI): 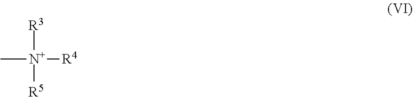
and the groups F bind to a carbon atom of R¹,
wherein
R³, R⁴, R⁵ are independently selected from optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups 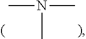
quaternary ammonium groups 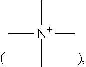
and can be substituted by OH,
the counter ions A⁻ are selected from mono- to trivalent inorganic anions and mono- to 30000-valent, organic anions
with the proviso that at least one of the radicals R¹, R³, R⁴, R⁵ of the cationic structure of the formulas (I) and (II) contains at least one moiety of the general formulas (VII) or (VIII):
—X—C(O)—R^{x}—(X—C(O)—R)ₘ₋₁—X—C(O)—R⁷ (VII) or
—X—C(O)—R^{x}—(X—C(O)—R^{x})ₘ—X—C(O)—R⁷ (VIII)
wherein
X is O or NR¹¹
m=1 to 20, and
the total number of carbon atoms in R^{x}+R⁷ (Σcarbon atoms R^{x}, R⁷) is 19 to 300,
R¹¹ is selected from the group consisting of hydrogen, n-, iso-, or tert.—C₁-C₂₂-alkyl
R^{x} is optionally OH, —O—C(O)—R⁷, —O—C(O)—R⁶—(O—C(O)—R⁶)₀₋₁₉—O—C(O)—R⁷ substituted straight-chain, cyclic or branched, saturated or unsaturated hydrocarbon radicals which have 1 to 36 carbon atoms, R⁶ is as defined,
R⁷ is optionally substituted straight-chain, cyclic or branched, saturated or unsaturated hydrocarbon radicals which have 1 to 36 carbon atoms.

51. The compound according to claim 42, wherein R¹ is selected from
poly(alkylene oxide) groups, of the general formula (IX):
—[CH₂CH₂O]_{q1}—[CH₂CH(CH₃)O]ᵣ₁—[CH₂CH(C₂H₅)O]ₛ₁—{[CH₂CH₂]_{q2}—[CH₂CH(CH₃)]ᵣ₂—[CH₂CH(C₂H₅)]ₛ₂}— (IX)
with
q1=0 to 49, r1=0 to 32, s1=0 to 24,
q2=0 or 1,
r2=0 or 1,
s2=0 or 1, and
Σ(q2+r2+s2)=1,
with the proviso that the sum of the carbon atoms in such poly(alkylene oxide) groups is 2 to 100, or R¹ is selected from
divalent hydrocarbon groups derived from oligoglycerols of the general formula (X):
—[CH₂CH(R⁸)CH₂O]ₜ₁—[CH₂CH(R⁸)CH₂)]ₜ₂— (X)
with
t1=0 to 32, t2=1,
R⁸=OH or (—X—C(O)—R⁶)ₘ—X—C(O)—R⁷, —O—C(O)—R⁶—N⁺(R³, R⁴, R⁵),
wherein m, X, R³, R⁴, R⁵, R⁶ and R⁷ are as defined,
with the proviso that the sum of the carbon atoms is 2 to 100, or R¹ is selected from
the divalent hydrocarbon groups comprising at least one ester group of the general formula (XI):
—[CH₂CH₂O]_{q1}—R⁹—[CH₂CH₂O]_{q1}—[CH₂CH₂]_{q2}— (XI)
with q1 being the same or different and being as defined, and q2=1,
and of the formula (XII)
—[CH₂CH(R⁸)CH₂O]ₜ₁—R⁹—[CH₂CH(R⁸)CH₂O]ₜ₁—[CH₂CH(R⁸)CH₂)]ₜ₂— (XII)
with t1, t2 and R⁸ as defined and
R⁹ being selected from —C(O)C(O)O—, —C(O)(CH₂)₁₋₈C(O)O—, or —C(O)(C₆H₄)C(O)O—, —C(O)CH═CHC(O)O—, —C(O)C(═CH₂)—CH₂C(O)O—, —C(O)CH(OH)CH(OH)C(O)O—,
with the proviso that the sum of the carbon atoms in R⁹ is 2 to 100.

52. The compound of claim 42, wherein
when one or more of the radicals R¹, R³, R⁴, R⁵ bonded to N⁺
contain the at least one moiety of the general formulas (III) or (IV)
(—X—C(O)—R⁶)ₘ—X—C(O)— (III) or
(—C(O)—X—R⁶)ₘ—C(O)—X— (IV),
with m=1-20 and X, R⁶ and R⁷ being as defined, or
the at least one moiety has the structure of the general formulas (XIII) or (XIV)
—R¹⁰(—X—C(O)—R⁶)ₘ—X—C(O)— (XIII) or
—R¹⁰(—C(O)—X—R⁶)ₘ—C(O)—X— (XIV),
wherein
R¹⁰ is selected from divalent to octadecavalent, optionally substituted hydrocarbon radicals which have up to 200 carbon atoms, and may contain optionally one or more groups selected from —O—, —NH—, —C(O)—, —C(S)—, tertiary amino groups 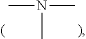
quaternary ammonium groups 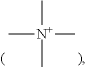
and can be substituted by —OH or halide groups, wherein the radical R¹⁰ cannot contain a combination of a —C(O)— group and a —O— group or a combination of a —C(O)— group and a —NH— or tertiary amino group forming an internal carboxylate group or an internal amide group,
with the proviso that R¹⁰ is linked by a single bond to a N⁺ moiety and is linked to at least one radical of the structures of the general formulas (III) or (IV)
(—X—C(O)—R⁶)ₘ—X—C(O)— (III) or
(—C(O)—X—R⁶)ₘ—C(O)—X— (IV),
with X, m, R¹¹, R⁶, R⁷ as defined.

53. The compound according claim 42
wherein low melting and high melting fatty acids≥C5 are specifically positioned within the R⁶ containing ester elements of the general formulas (III) and (IV)
(—X—C(O)—R⁶)ₘ—X—C(O)— (III)
(—C(O)—X—R⁶)ₘ—C(O)—X— (IV), or
within the R⁶ containing ester elements of the general formulas (XIII) and (XIV)
R¹⁰(—X—C(O)—R⁶)ₘ—X—C(O)— (XIII)
—R¹⁰(—C(O)—X—R⁶)ₘ—C(O)—X— (XIV),
with low melting fatty acids≥C5 being defined by a melting point≤40° C. and high melting fatty acids≥C5 being defined by a melting point>40° C., in such a manner that
at least one, low melting fatty acids≥C5 each forming a group R⁶ are positioned at the one terminus of a R⁶-containing ester element of the formula (III) or (IV), while at least one high melting fatty acids≥C5 form the radical or radicals R⁶ at the opposite terminus of the ester element of the formulas (III) or (IV), or in such a manner that at least one high melting fatty acids≥C5 each forming a group R⁶ are positioned at the one terminus of a R⁶-containing ester element of the formula (III) or (IV), while at least one, low melting fatty acids≥C5 form the radical or radicals R⁶ at the opposite terminus of the ester element of the formula (III) or (IV), or
at least one, low melting fatty acids≥C5 each forming a group R⁶ are contained in the radical or the radicals R⁶ adjacent to R⁷, while at least one, high melting fatty acids≥C5 form the radical or radicals R⁶ at the opposite terminus of a R⁶- and R⁷-containing ester element of the formula (IIIa) or (IVa), or in such a manner that least one, high melting fatty acids≥C5 each forming R⁶ form the radical or radicals R⁶ adjacent to R⁷, while at least one, low melting fatty acids≥C5 form the radical or radicals R⁶ at the opposite terminus of a R⁶- and R⁷-containing ester element of the formulas (IIIa) or (IVa), or
at least one, low melting fatty acids≥C5 each forming a group R⁶ are positioned adjacent to the radical R¹⁰ while at least one, high melting fatty acids≥C5 form the radical or the radicals R⁶ at the opposite terminus of the ester element of the formula (XIII) or (XIV), or in such a manner that least one, high melting fatty acids≥C5 each forming R⁶ form the radical or radicals R⁶ adjacent to the radical R¹⁰, while at least one, low melting fatty acids≥C5 form the radical or radicals R⁶ at the opposite terminus of a R⁶- and R⁷-containing ester element of the formula (XIII) or (XIV), or
at least one, low melting fatty acids≥C5 each forming a group R⁶ are positioned adjacent to the radical R¹⁰ while at least one, high melting fatty acids≥C5 form the radical or radicals R⁶ adjacent to R⁷ in the moieties of the formulas (XIIIa) or (XIVa), or in such a manner that at least one, high melting fatty acids≥C5 each forming R⁶ form the radical or radicals R⁶ adjacent to the radical R¹⁰ while at least one, low melting fatty acids≥C5 form the radical or radicals R⁶ adjacent to R⁷ in the moieties of the formulas (XIIIa) or (XIVa).

54. The compound according to claim 42, wherein
the counter ions A⁻ are mono- to trivalent inorganic anions and mono- to 30000-valent, organic anions selected from the group consisting of halide anions, sulphate, phosphate, phosphonate, sulphonate, methosulphate, carboxylate anions, polyethercarboxylate,
polymeric fatty acid carboxylates of the type
R¹[(—C(O)—X—R⁶)ₘ—C(O)—X—R⁷]ₓ or
R¹[(X—C(O)—R⁶)ₘ—X—C(O)—R⁷]ₓ, wherein either R¹ or at least one of R⁷, or both R¹ and at least one of R⁷ bear one or more carboxylate groups,
wherein X, R¹, R⁶, R⁷, m and x are as defined and wherein the counter ions A⁻ of this group are mono- to pentacontavalent, anions,
or the group consisting of poly (acrylic acid) homo and copolymers, poly (itaconic acid) homo and copolymers, as defined
being branched or dendrimeric (self repeating) motif-containing carboxylates.

55. The compound according to claim 42,
wherein in Formula (III) and/or (IV) X=O.

56. The compound according to claim 42, wherein at least one of the groups R¹, R², R³, R⁴, R⁵ present in the cationic structure of the general formulas (I) and (II) contains at least one moiety of the formula
R^{1*}[(—O—C(O)—R⁶)ₘ—O—C(O)—]₂,
wherein R^{1*} is a divalent C1-C100 radical, m is independently selected from 1 to 12, and R⁶ is as defined above.

57. The compound of claim 42, wherein in at least one moiety of the general formula R^{1*}[(—O—C(O)—R⁶)ₘ—O—C(O)—]₂,
R^{1*} is selected from methylene, ethylene, 1,3-propylene, 1,4-butylene, 1,6-hexylene, 1,2-propylene, 1,3-butylene,
R⁶ is derived from C8-C24 monocarboxy-monohydroxy carboxylic acids,
and m is independently selected from 1 to 6.

58. The compound according to claim 42, wherein at least one of the groups R¹, R², R³, R⁴, R⁵ present in the cationic structure of the general formulas (I) and (II) contains at least one moiety of the formula
R^{1*}[(—O—C(O)—R⁶)ₘ—O—C(O)—R⁷*—]₂,
wherein R^{1*}, R⁶, and m are as defined,
and R^{7*} is a C1-C12 alkylene group.

59. The compound according to claim 56, wherein at least one moiety of the general formula
R^{1*}[(—O—C(O)—R⁶)ₘ—O—C(O)—R⁷*—]₂
is bonded to a quaternary N atom on one or both terminal R^{7*} groups.

60. The compound according to claim 59, wherein both terminal groups R⁷* are each bonded to a quaternary N atom,
and wherein the compound is a di-quat or a tetra-quat compound.

61. The compound according to claim 42, wherein at least one of the groups R¹, R², R³, R⁴, R⁵ present in the cationic structure of the general formulas (I) and (II) contains at least one moiety of the general formula
—([—O—C(O)—R⁶(—O—C(O)—R⁶)ₗ—O—C(O)-L-C(O)—O—(R⁶—C(O)—O)ₗ—R⁶—C(O)O])—
wherein R⁶ is as defined above,
l is an integer independently selected from 0-20, and
L is a divalent hydrocarbon radical which may have 1 to 30 carbon atoms and may contain optionally one or more groups selected from —O—, —S—, —NH—, —C(O)—, —C(S)—, and tertiary amino groups

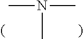

62. The compound according to claim 61, wherein in at least one moiety of the general formula
—([—O—C(O)—R⁶(—O—C(O)—R⁶)ₗ—O—C(O)-L-C(O)—O—(R⁶—C(O)—O)ₗ—R⁶—C(O)O])—
L and l are as defined,
and R⁶ is independently derived from C8-C24 monocarboxy-monohydroxy carboxylic acids.

63. A process for the synthesis of the compounds of the general formula (I)
R¹(—F)ₓ (I)
according to claim 42, wherein
alkyl halogenides are reacted with tertiary amines containing at least one moiety
(—X—C(O)—R⁶)ₘ—X—C(O)— (III) or
(—C(O)—X—R⁶)ₘ—C(O)—X— (IV),
or
esters of halogen carboxylic acids, with alcohols or epoxides, as defined, are reacted with tertiary amines containing at least one moiety
(—X—C(O)—R⁶)ₘ—X—C(O)— (III) or
(—C(O)—X—R⁶)ₘ—C(O)—X— (IV),
or
epoxy functionalized ethers and esters, with alcohols or carboxylic acids, as defined, are reacted with tertiary amines containing at least one moiety
(—X—C(O)—R⁶)ₘ—X—C(O)— (III) or
(—C(O)—X—R⁶)ₘ—C(O)—X— (IV), in the presence of an acid,
or
tertiary amino groups containing hydrocarbons are reacted with esters of halogen carboxylic acids, as defined, containing at least one moiety
(—X—C(O)—R⁶)ₘ—X—C(O)— (III) or
(—C(O)—X—R⁶)ₘ—C(O)—X— (IV),
or
tertiary amino groups containing hydrocarbons are reacted with epoxy functionalized ethers and esters, as defined, containing at least one moiety
(—X—C(O)—R⁶)ₘ—X—C(O)— (III) or
(—C(O)—X—R⁶)ₘ—C(O)—X— (IV),
in the presence of an acid,
wherein X, R⁶, R⁷, m and x are as defined.

64. A process for the synthesis of compounds of the general formula (I) according to claim 63, wherein
for compounds of the general formula (I)
R¹(—F)ₓ (I),
with R¹ being linked through a quaternized nitrogen atom N⁺ to R³, R⁴, and R⁵, and R¹(—F)ₓ containing at least one moiety of the general formula (III)
(—X—C(O)—R⁶)ₘ—X—C(O)— (III),
or of the general formula (IV)
(—C(O)—X—R⁶)ₘ—C(O)—X— (IV),
alkyl halogenides are reacted with tertiary amines containing at least one moiety
(—X—C(O)—R⁶)ₘ—X—C(O)— (III) or
(—C(O)—X—R⁶)ₘ—C(O)—X— (IV),
or
esters of halogen carboxylic acids, with alcohols or epoxides, as defined, are reacted with tertiary amines containing at least one moiety
(—X—C(O)—R⁶)ₘ—X—C(O)— (III) or
(—C(O)—X—R⁶)ₘ—C(O)—X— (IV),
or
epoxy functionalized ethers and esters, with alcohols or carboxylic acids, as defined, are reacted with tertiary amines containing at least one moiety
(—X—C(O)—R⁶)ₘ—X—C(O)— (III) or
(—C(O)—X—R⁶)ₘ—C(O)—X— (IV), in the presence of an acid,
or
tertiary amino groups containing hydrocarbons are reacted with esters of halogen carboxylic acids, as defined, containing at least one moiety
(—X—C(O)—R⁶)ₘ—X—C(O)— (III) or
(—C(O)—X—R⁶)ₘ—C(O)—X— (IV),
or
tertiary amino groups containing hydrocarbons are reacted with epoxy functionalized ethers and esters, as defined, containing at least one moiety
(—X—C(O)—R⁶)ₘ—X—C(O)— (III) or
(—C(O)—X—R⁶)ₘ—C(O)—X— (IV),
in the presence of an acid.

65. A cosmetic formulation for skin and hair care, a polishing agent for treating and coating hard surfaces, a formulation for drying automobiles and other hard surfaces, a formulation for finishing textiles and textile fibers, as separate softeners for use after textiles have been washed with nonionic or anionic/nonionic detergent formulations, as softeners in formulations for washing textiles that are based upon nonionic or anionic/nonionic surfactants, and as means for preventing or removing wrinkles in textiles, which comprise the compound of claim 42.

66. A cosmetic composition for the treatment of fibers, comprising the compound of claim 42.
